SEQUENCE LISTING (1366 sequences)

SEQ ID NO: 1 - protein, 18 aa, Artificial Sequence
  Synthetic construct
GTYGLSSPGSPGTPGSRC

SEQ ID NO: 2 - protein, 19 aa, Artificial Sequence
  Synthetic construct
GTYGLYSSPGSPGTPGSRC

SEQ ID NO: 3 - protein, 19 aa, Artificial Sequence
  Synthetic construct
GTYGLSSPGSPGTPGSRSC

SEQ ID NO: 4 - protein, 20 aa, Artificial Sequence
  Synthetic construct
GTYGLGYSSPGSPGTPGSRC

SEQ ID NO: 5 - protein, 20 aa, Artificial Sequence
  Synthetic construct
GTYGLYSSPGSPGTPGSRSC

SEQ ID NO: 6 - protein, 20 aa, Artificial Sequence
  Synthetic construct
GTYGLSSPGSPGTPGSRSRC

SEQ ID NO: 7 - protein, 21 aa, Artificial Sequence
  Synthetic construct
GTYGLSGYSSPGSPGTPGSRC

SEQ ID NO: 8 - protein, 21 aa, Artificial Sequence
  Synthetic construct
GTYGLGYSSPGSPGTPGSRSC

SEQ ID NO: 9 - protein, 21 aa, Artificial Sequence
  Synthetic construct
GTYGLYSSPGSPGTPGSRSRC

SEQ ID NO: 10 - protein, 21 aa, Artificial Sequence
  Synthetic construct
GTYGLSSPGSPGTPGSRSRTC

SEQ ID NO: 11 - protein, 22 aa, Artificial Sequence
  Synthetic construct
GTYGLRSGYSSPGSPGTPGSRC

SEQ ID NO: 12 - protein, 22 aa, Artificial Sequence
  Synthetic construct
GTYGLSGYSSPGSPGTPGSRSC

SEQ ID NO: 13 - protein, 22 aa, Artificial Sequence
  Synthetic construct
GTYGLGYSSPGSPGTPGSRSRC

SEQ ID NO: 14 - protein, 22 aa, Artificial Sequence
  Synthetic construct
GTYGLYSSPGSPGTPGSRSRTC

SEQ ID NO: 15 - protein, 22 aa, Artificial Sequence
  Synthetic construct
GTYGLSSPGSPGTPGSRSRTPC

SEQ ID NO: 16 - protein, 23 aa, Artificial Sequence
  Synthetic construct
GTYGLRSGYSSPGSPGTPGSRSC

SEQ ID NO: 17 - protein, 23 aa, Artificial Sequence
  Synthetic construct
GTYGLSGYSSPGSPGTPGSRSRC

SEQ ID NO: 18 - protein, 23 aa, Artificial Sequence
  Synthetic construct
GTYGLGYSSPGSPGTPGSRSRTC

SEQ ID NO: 19 - protein, 23 aa, Artificial Sequence
  Synthetic construct
GTYGLYSSPGSPGTPGSRSRTPC

SEQ ID NO: 20 - protein, 24 aa, Artificial Sequence
  Synthetic construct
GTYGLRSGYSSPGSPGTPGSRSRC

SEQ ID NO: 21 - protein, 24 aa, Artificial Sequence
  Synthetic construct
GTYGLSGYSSPGSPGTPGSRSRTC

SEQ ID NO: 22 - protein, 24 aa, Artificial Sequence
  Synthetic construct
GTYGLGYSSPGSPGTPGSRSRTPC

SEQ ID NO: 23 - protein, 25 aa, Artificial Sequence
  Synthetic construct
GTYGLRSGYSSPGSPGTPGSRSRTC

SEQ ID NO: 24 - protein, 25 aa, Artificial Sequence
  Synthetic construct
GTYGLSGYSSPGSPGTPGSRSRTPC

SEQ ID NO: 25 - protein, 26 aa, Artificial Sequence
  Synthetic construct
GTYGLRSGYSSPGSPGTPGSRSRTPC

SEQ ID NO: 26 - protein, 19 aa, Artificial Sequence
  Synthetic construct
AGTYGLSSPGSPGTPGSRC

SEQ ID NO: 27 - protein, 20 aa, Artificial Sequence
  Synthetic construct
AGTYGLYSSPGSPGTPGSRC

SEQ ID NO: 28 - protein, 20 aa, Artificial Sequence
  Synthetic construct
AGTYGLSSPGSPGTPGSRSC

SEQ ID NO: 29 - protein, 21 aa, Artificial Sequence
  Synthetic construct
AGTYGLGYSSPGSPGTPGSRC

SEQ ID NO: 30 - protein, 21 aa, Artificial Sequence
  Synthetic construct
AGTYGLYSSPGSPGTPGSRSC

SEQ ID NO: 31 - protein, 21 aa, Artificial Sequence
  Synthetic construct
AGTYGLSSPGSPGTPGSRSRC

SEQ ID NO: 32 - protein, 22 aa, Artificial Sequence
  Synthetic construct
AGTYGLSGYSSPGSPGTPGSRC

SEQ ID NO: 33 - protein, 22 aa, Artificial Sequence
  Synthetic construct
AGTYGLGYSSPGSPGTPGSRSC

SEQ ID NO: 34 - protein, 22 aa, Artificial Sequence
  Synthetic construct
AGTYGLYSSPGSPGTPGSRSRC

SEQ ID NO: 35 - protein, 22 aa, Artificial Sequence
  Synthetic construct
AGTYGLSSPGSPGTPGSRSRTC

SEQ ID NO: 36 - protein, 23 aa, Artificial Sequence
  Synthetic construct
AGTYGLRSGYSSPGSPGTPGSRC

SEQ ID NO: 37 - protein, 23 aa, Artificial Sequence
  Synthetic construct
AGTYGLSGYSSPGSPGTPGSRSC

SEQ ID NO: 38 - protein, 23 aa, Artificial Sequence
  Synthetic construct
AGTYGLGYSSPGSPGTPGSRSRC

SEQ ID NO: 39 - protein, 23 aa, Artificial Sequence
  Synthetic construct
AGTYGLYSSPGSPGTPGSRSRTC

SEQ ID NO: 40 - protein, 23 aa, Artificial Sequence
  Synthetic construct
AGTYGLSSPGSPGTPGSRSRTPC

SEQ ID NO: 41 - protein, 24 aa, Artificial Sequence
  Synthetic construct
AGTYGLRSGYSSPGSPGTPGSRSC

SEQ ID NO: 42 - protein, 24 aa, Artificial Sequence
  Synthetic construct
AGTYGLSGYSSPGSPGTPGSRSRC

SEQ ID NO: 43 - protein, 24 aa, Artificial Sequence
  Synthetic construct
AGTYGLGYSSPGSPGTPGSRSRTC

SEQ ID NO: 44 - protein, 24 aa, Artificial Sequence
  Synthetic construct
AGTYGLYSSPGSPGTPGSRSRTPC

SEQ ID NO: 45 - protein, 25 aa, Artificial Sequence
  Synthetic construct
AGTYGLRSGYSSPGSPGTPGSRSRC

SEQ ID NO: 46 - protein, 25 aa, Artificial Sequence
  Synthetic construct
AGTYGLSGYSSPGSPGTPGSRSRTC

SEQ ID NO: 47 - protein, 25 aa, Artificial Sequence
  Synthetic construct
AGTYGLGYSSPGSPGTPGSRSRTPC

SEQ ID NO: 48 - protein, 26 aa, Artificial Sequence
  Synthetic construct
AGTYGLRSGYSSPGSPGTPGSRSRTC

SEQ ID NO: 49 - protein, 26 aa, Artificial Sequence
  Synthetic construct
AGTYGLSGYSSPGSPGTPGSRSRTPC

SEQ ID NO: 50 - protein, 27 aa, Artificial Sequence
  Synthetic construct
AGTYGLRSGYSSPGSPGTPGSRSRTPC

SEQ ID NO: 51 - protein, 19 aa, Artificial Sequence
  Synthetic construct
GTYGLGSSPGSPGTPGSRC

SEQ ID NO: 52 - protein, 20 aa, Artificial Sequence
  Synthetic construct
GTYGLGYSSPGSPGTPGSRC

SEQ ID NO: 53 - protein, 20 aa, Artificial Sequence
  Synthetic construct
GTYGLGSSPGSPGTPGSRSC

SEQ ID NO: 54 - protein, 21 aa, Artificial Sequence
  Synthetic construct
GTYGLGGYSSPGSPGTPGSRC

SEQ ID NO: 55 - protein, 21 aa, Artificial Sequence
  Synthetic construct
GTYGLGYSSPGSPGTPGSRSC

SEQ ID NO: 56 - protein, 21 aa, Artificial Sequence
  Synthetic construct
GTYGLGSSPGSPGTPGSRSRC

SEQ ID NO: 57 - protein, 22 aa, Artificial Sequence
  Synthetic construct
GTYGLGSGYSSPGSPGTPGSRC

SEQ ID NO: 58 - protein, 22 aa, Artificial Sequence
  Synthetic construct
GTYGLGGYSSPGSPGTPGSRSC

SEQ ID NO: 59 - protein, 22 aa, Artificial Sequence
  Synthetic construct
GTYGLGYSSPGSPGTPGSRSRC

SEQ ID NO: 60 - protein, 22 aa, Artificial Sequence
  Synthetic construct
GTYGLGSSPGSPGTPGSRSRTC

SEQ ID NO: 61 - protein, 23 aa, Artificial Sequence
  Synthetic construct
GTYGLGRSGYSSPGSPGTPGSRC

SEQ ID NO: 62 - protein, 23 aa, Artificial Sequence
  Synthetic construct
GTYGLGSGYSSPGSPGTPGSRSC

SEQ ID NO: 63 - protein, 23 aa, Artificial Sequence
  Synthetic construct
GTYGLGGYSSPGSPGTPGSRSRC

SEQ ID NO: 64 - protein, 23 aa, Artificial Sequence
  Synthetic construct
GTYGLGYSSPGSPGTPGSRSRTC

SEQ ID NO: 65 - protein, 23 aa, Artificial Sequence
  Synthetic construct
GTYGLGSSPGSPGTPGSRSRTPC

SEQ ID NO: 66 - protein, 24 aa, Artificial Sequence
  Synthetic construct
GTYGLGRSGYSSPGSPGTPGSRSC

SEQ ID NO: 67 - protein, 24 aa, Artificial Sequence
  Synthetic construct
GTYGLGSGYSSPGSPGTPGSRSRC

SEQ ID NO: 68 - protein, 24 aa, Artificial Sequence
  Synthetic construct
GTYGLGGYSSPGSPGTPGSRSRTC

SEQ ID NO: 69 - protein, 24 aa, Artificial Sequence
  Synthetic construct
GTYGLGYSSPGSPGTPGSRSRTPC

SEQ ID NO: 70 - protein, 25 aa, Artificial Sequence
  Synthetic construct
GTYGLGRSGYSSPGSPGTPGSRSRC

SEQ ID NO: 71 - protein, 25 aa, Artificial Sequence
  Synthetic construct
GTYGLGSGYSSPGSPGTPGSRSRTC

SEQ ID NO: 72 - protein, 25 aa, Artificial Sequence
  Synthetic construct
GTYGLGGYSSPGSPGTPGSRSRTPC

SEQ ID NO: 73 - protein, 26 aa, Artificial Sequence
  Synthetic construct
GTYGLGRSGYSSPGSPGTPGSRSRTC

SEQ ID NO: 74 - protein, 26 aa, Artificial Sequence
  Synthetic construct
GTYGLGSGYSSPGSPGTPGSRSRTPC

SEQ ID NO: 75 - protein, 27 aa, Artificial Sequence
  Synthetic construct
GTYGLGRSGYSSPGSPGTPGSRSRTPC

SEQ ID NO: 76 - protein, 20 aa, Artificial Sequence
  Synthetic construct
HAGTYGLSSPGSPGTPGSRC

SEQ ID NO: 77 - protein, 21 aa, Artificial Sequence
  Synthetic construct
HAGTYGLYSSPGSPGTPGSRC

SEQ ID NO: 78 - protein, 21 aa, Artificial Sequence
  Synthetic construct
HAGTYGLSSPGSPGTPGSRSC

SEQ ID NO: 79 - protein, 22 aa, Artificial Sequence
  Synthetic construct
HAGTYGLGYSSPGSPGTPGSRC

SEQ ID NO: 80 - protein, 22 aa, Artificial Sequence
  Synthetic construct
HAGTYGLYSSPGSPGTPGSRSC

SEQ ID NO: 81 - protein, 22 aa, Artificial Sequence
  Synthetic construct
HAGTYGLSSPGSPGTPGSRSRC

SEQ ID NO: 82 - protein, 23 aa, Artificial Sequence
  Synthetic construct
HAGTYGLSGYSSPGSPGTPGSRC

SEQ ID NO: 83 - protein, 23 aa, Artificial Sequence
  Synthetic construct
HAGTYGLGYSSPGSPGTPGSRSC

SEQ ID NO: 84 - protein, 23 aa, Artificial Sequence
  Synthetic construct
HAGTYGLYSSPGSPGTPGSRSRC

SEQ ID NO: 85 - protein, 23 aa, Artificial Sequence
  Synthetic construct
HAGTYGLSSPGSPGTPGSRSRTC

SEQ ID NO: 86 - protein, 24 aa, Artificial Sequence
  Synthetic construct
HAGTYGLRSGYSSPGSPGTPGSRC

SEQ ID NO: 87 - protein, 24 aa, Artificial Sequence
  Synthetic construct
HAGTYGLSGYSSPGSPGTPGSRSC

SEQ ID NO: 88 - protein, 24 aa, Artificial Sequence
  Synthetic construct
HAGTYGLGYSSPGSPGTPGSRSRC

SEQ ID NO: 89 - protein, 24 aa, Artificial Sequence
  Synthetic construct
HAGTYGLYSSPGSPGTPGSRSRTC

SEQ ID NO: 90 - protein, 24 aa, Artificial Sequence
  Synthetic construct
HAGTYGLSSPGSPGTPGSRSRTPC

SEQ ID NO: 91 - protein, 25 aa, Artificial Sequence
  Synthetic construct
HAGTYGLRSGYSSPGSPGTPGSRSC

SEQ ID NO: 92 - protein, 25 aa, Artificial Sequence
  Synthetic construct
HAGTYGLSGYSSPGSPGTPGSRSRC

SEQ ID NO: 93 - protein, 25 aa, Artificial Sequence
  Synthetic construct
HAGTYGLGYSSPGSPGTPGSRSRTC

SEQ ID NO: 94 - protein, 25 aa, Artificial Sequence
  Synthetic construct
HAGTYGLYSSPGSPGTPGSRSRTPC

SEQ ID NO: 95 - protein, 26 aa, Artificial Sequence
  Synthetic construct
HAGTYGLRSGYSSPGSPGTPGSRSRC

SEQ ID NO: 96 - protein, 26 aa, Artificial Sequence
  Synthetic construct
HAGTYGLSGYSSPGSPGTPGSRSRTC

SEQ ID NO: 97 - protein, 26 aa, Artificial Sequence
  Synthetic construct
HAGTYGLGYSSPGSPGTPGSRSRTPC

SEQ ID NO: 98 - protein, 27 aa, Artificial Sequence
  Synthetic construct
HAGTYGLRSGYSSPGSPGTPGSRSRTC

SEQ ID NO: 99 - protein, 27 aa, Artificial Sequence
  Synthetic construct
HAGTYGLSGYSSPGSPGTPGSRSRTPC

SEQ ID NO: 100 - protein, 28 aa, Artificial Sequence
  Synthetic construct
HAGTYGLRSGYSSPGSPGTPGSRSRTPC

SEQ ID NO: 101 - protein, 20 aa, Artificial Sequence
  Synthetic construct
AGTYGLGSSPGSPGTPGSRC

SEQ ID NO: 102 - protein, 21 aa, Artificial Sequence
  Synthetic construct
AGTYGLGYSSPGSPGTPGSRC

SEQ ID NO: 103 - protein, 21 aa, Artificial Sequence
  Synthetic construct
AGTYGLGSSPGSPGTPGSRSC

SEQ ID NO: 104 - protein, 22 aa, Artificial Sequence
  Synthetic construct
AGTYGLGGYSSPGSPGTPGSRC

SEQ ID NO: 105 - protein, 22 aa, Artificial Sequence
  Synthetic construct
AGTYGLGYSSPGSPGTPGSRSC

SEQ ID NO: 106 - protein, 22 aa, Artificial Sequence
  Synthetic construct
AGTYGLGSSPGSPGTPGSRSRC

SEQ ID NO: 107 - protein, 23 aa, Artificial Sequence
  Synthetic construct
AGTYGLGSGYSSPGSPGTPGSRC

SEQ ID NO: 108 - protein, 23 aa, Artificial Sequence
  Synthetic construct
AGTYGLGGYSSPGSPGTPGSRSC

SEQ ID NO: 109 - protein, 23 aa, Artificial Sequence
  Synthetic construct
AGTYGLGYSSPGSPGTPGSRSRC

SEQ ID NO: 110 - protein, 23 aa, Artificial Sequence
  Synthetic construct
AGTYGLGSSPGSPGTPGSRSRTC

SEQ ID NO: 111 - protein, 24 aa, Artificial Sequence
  Synthetic construct
AGTYGLGRSGYSSPGSPGTPGSRC

SEQ ID NO: 112 - protein, 24 aa, Artificial Sequence
  Synthetic construct
AGTYGLGSGYSSPGSPGTPGSRSC

SEQ ID NO: 113 - protein, 24 aa, Artificial Sequence
  Synthetic construct
AGTYGLGGYSSPGSPGTPGSRSRC

SEQ ID NO: 114 - protein, 24 aa, Artificial Sequence
  Synthetic construct
AGTYGLGYSSPGSPGTPGSRSRTC

SEQ ID NO: 115 - protein, 24 aa, Artificial Sequence
  Synthetic construct
AGTYGLGSSPGSPGTPGSRSRTPC

SEQ ID NO: 116 - protein, 25 aa, Artificial Sequence
  Synthetic construct
AGTYGLGRSGYSSPGSPGTPGSRSC

SEQ ID NO: 117 - protein, 25 aa, Artificial Sequence
  Synthetic construct
AGTYGLGSGYSSPGSPGTPGSRSRC

SEQ ID NO: 118 - protein, 25 aa, Artificial Sequence
  Synthetic construct
AGTYGLGGYSSPGSPGTPGSRSRTC

SEQ ID NO: 119 - protein, 25 aa, Artificial Sequence
  Synthetic construct
AGTYGLGYSSPGSPGTPGSRSRTPC

SEQ ID NO: 120 - protein, 26 aa, Artificial Sequence
  Synthetic construct
AGTYGLGRSGYSSPGSPGTPGSRSRC

SEQ ID NO: 121 - protein, 26 aa, Artificial Sequence
  Synthetic construct
AGTYGLGSGYSSPGSPGTPGSRSRTC

SEQ ID NO: 122 - protein, 26 aa, Artificial Sequence
  Synthetic construct
AGTYGLGGYSSPGSPGTPGSRSRTPC

SEQ ID NO: 123 - protein, 27 aa, Artificial Sequence
  Synthetic construct
AGTYGLGRSGYSSPGSPGTPGSRSRTC

SEQ ID NO: 124 - protein, 27 aa, Artificial Sequence
  Synthetic construct
AGTYGLGSGYSSPGSPGTPGSRSRTPC

SEQ ID NO: 125 - protein, 28 aa, Artificial Sequence
  Synthetic construct
AGTYGLGRSGYSSPGSPGTPGSRSRTPC

SEQ ID NO: 126 - protein, 20 aa, Artificial Sequence
  Synthetic construct
GTYGLGDSSPGSPGTPGSRC

SEQ ID NO: 127 - protein, 21 aa, Artificial Sequence
  Synthetic construct
GTYGLGDYSSPGSPGTPGSRC

SEQ ID NO: 128 - protein, 21 aa, Artificial Sequence
  Synthetic construct
GTYGLGDSSPGSPGTPGSRSC

SEQ ID NO: 129 - protein, 22 aa, Artificial Sequence
  Synthetic construct
GTYGLGDGYSSPGSPGTPGSRC

SEQ ID NO: 130 - protein, 22 aa, Artificial Sequence
  Synthetic construct
GTYGLGDYSSPGSPGTPGSRSC

SEQ ID NO: 131 - protein, 22 aa, Artificial Sequence
  Synthetic construct
GTYGLGDSSPGSPGTPGSRSRC

SEQ ID NO: 132 - protein, 23 aa, Artificial Sequence
  Synthetic construct
GTYGLGDSGYSSPGSPGTPGSRC

SEQ ID NO: 133 - protein, 23 aa, Artificial Sequence
  Synthetic construct
GTYGLGDGYSSPGSPGTPGSRSC

SEQ ID NO: 134 - protein, 23 aa, Artificial Sequence
  Synthetic construct
GTYGLGDYSSPGSPGTPGSRSRC

SEQ ID NO: 135 - protein, 23 aa, Artificial Sequence
  Synthetic construct
GTYGLGDSSPGSPGTPGSRSRTC

SEQ ID NO: 136 - protein, 24 aa, Artificial Sequence
  Synthetic construct
GTYGLGDRSGYSSPGSPGTPGSRC

SEQ ID NO: 137 - protein, 24 aa, Artificial Sequence
  Synthetic construct
GTYGLGDSGYSSPGSPGTPGSRSC

SEQ ID NO: 138 - protein, 24 aa, Artificial Sequence
  Synthetic construct
GTYGLGDGYSSPGSPGTPGSRSRC

SEQ ID NO: 139 - protein, 24 aa, Artificial Sequence
  Synthetic construct
GTYGLGDYSSPGSPGTPGSRSRTC

SEQ ID NO: 140 - protein, 24 aa, Artificial Sequence
  Synthetic construct
GTYGLGDSSPGSPGTPGSRSRTPC

SEQ ID NO: 141 - protein, 25 aa, Artificial Sequence
  Synthetic construct
GTYGLGDRSGYSSPGSPGTPGSRSC

SEQ ID NO: 142 - protein, 25 aa, Artificial Sequence
  Synthetic construct
GTYGLGDSGYSSPGSPGTPGSRSRC

SEQ ID NO: 143 - protein, 25 aa, Artificial Sequence
  Synthetic construct
GTYGLGDGYSSPGSPGTPGSRSRTC

SEQ ID NO: 144 - protein, 25 aa, Artificial Sequence
  Synthetic construct
GTYGLGDYSSPGSPGTPGSRSRTPC

SEQ ID NO: 145 - protein, 26 aa, Artificial Sequence
  Synthetic construct
GTYGLGDRSGYSSPGSPGTPGSRSRC

SEQ ID NO: 146 - protein, 26 aa, Artificial Sequence
  Synthetic construct
GTYGLGDSGYSSPGSPGTPGSRSRTC

SEQ ID NO: 147 - protein, 26 aa, Artificial Sequence
  Synthetic construct
GTYGLGDGYSSPGSPGTPGSRSRTPC

SEQ ID NO: 148 - protein, 27 aa, Artificial Sequence
  Synthetic construct
GTYGLGDRSGYSSPGSPGTPGSRSRTC

SEQ ID NO: 149 - protein, 27 aa, Artificial Sequence
  Synthetic construct
GTYGLGDSGYSSPGSPGTPGSRSRTPC

SEQ ID NO: 150 - protein, 28 aa, Artificial Sequence
  Synthetic construct
GTYGLGDRSGYSSPGSPGTPGSRSRTPC

SEQ ID NO: 151 - protein, 21 aa, Artificial Sequence
  Synthetic construct
HAGTYGLGSSPGSPGTPGSRC

SEQ ID NO: 152 - protein, 22 aa, Artificial Sequence
  Synthetic construct
HAGTYGLGYSSPGSPGTPGSRC

SEQ ID NO: 153 - protein, 22 aa, Artificial Sequence
  Synthetic construct
HAGTYGLGSSPGSPGTPGSRSC

SEQ ID NO: 154 - protein, 23 aa, Artificial Sequence
  Synthetic construct
HAGTYGLGGYSSPGSPGTPGSRC

SEQ ID NO: 155 - protein, 23 aa, Artificial Sequence
  Synthetic construct
HAGTYGLGYSSPGSPGTPGSRSC

SEQ ID NO: 156 - protein, 23 aa, Artificial Sequence
  Synthetic construct
HAGTYGLGSSPGSPGTPGSRSRC

SEQ ID NO: 157 - protein, 24 aa, Artificial Sequence
  Synthetic construct
HAGTYGLGSGYSSPGSPGTPGSRC

SEQ ID NO: 158 - protein, 24 aa, Artificial Sequence
  Synthetic construct
HAGTYGLGGYSSPGSPGTPGSRSC

SEQ ID NO: 159 - protein, 24 aa, Artificial Sequence
  Synthetic construct
HAGTYGLGYSSPGSPGTPGSRSRC

SEQ ID NO: 160 - protein, 24 aa, Artificial Sequence
  Synthetic construct
HAGTYGLGSSPGSPGTPGSRSRTC

SEQ ID NO: 161 - protein, 25 aa, Artificial Sequence
  Synthetic construct
HAGTYGLGRSGYSSPGSPGTPGSRC

SEQ ID NO: 162 - protein, 25 aa, Artificial Sequence
  Synthetic construct
HAGTYGLGSGYSSPGSPGTPGSRSC

SEQ ID NO: 163 - protein, 25 aa, Artificial Sequence
  Synthetic construct
HAGTYGLGGYSSPGSPGTPGSRSRC

SEQ ID NO: 164 - protein, 25 aa, Artificial Sequence
  Synthetic construct
HAGTYGLGYSSPGSPGTPGSRSRTC

SEQ ID NO: 165 - protein, 25 aa, Artificial Sequence
  Synthetic construct
HAGTYGLGSSPGSPGTPGSRSRTPC

SEQ ID NO: 166 - protein, 26 aa, Artificial Sequence
  Synthetic construct
HAGTYGLGRSGYSSPGSPGTPGSRSC

SEQ ID NO: 167 - protein, 26 aa, Artificial Sequence
  Synthetic construct
HAGTYGLGSGYSSPGSPGTPGSRSRC

SEQ ID NO: 168 - protein, 26 aa, Artificial Sequence
  Synthetic construct
HAGTYGLGGYSSPGSPGTPGSRSRTC

SEQ ID NO: 169 - protein, 26 aa, Artificial Sequence
  Synthetic construct
HAGTYGLGYSSPGSPGTPGSRSRTPC

SEQ ID NO: 170 - protein, 27 aa, Artificial Sequence
  Synthetic construct
HAGTYGLGRSGYSSPGSPGTPGSRSRC

SEQ ID NO: 171 - protein, 27 aa, Artificial Sequence
  Synthetic construct
HAGTYGLGSGYSSPGSPGTPGSRSRTC

SEQ ID NO: 172 - protein, 27 aa, Artificial Sequence
  Synthetic construct
HAGTYGLGGYSSPGSPGTPGSRSRTPC

SEQ ID NO: 173 - protein, 28 aa, Artificial Sequence
  Synthetic construct
HAGTYGLGRSGYSSPGSPGTPGSRSRTC

SEQ ID NO: 174 - protein, 28 aa, Artificial Sequence
  Synthetic construct
HAGTYGLGSGYSSPGSPGTPGSRSRTPC

SEQ ID NO: 175 - protein, 29 aa, Artificial Sequence
  Synthetic construct
HAGTYGLGRSGYSSPGSPGTPGSRSRTPC

SEQ ID NO: 176 - protein, 21 aa, Artificial Sequence
  Synthetic construct
AGTYGLGDSSPGSPGTPGSRC

SEQ ID NO: 177 - protein, 22 aa, Artificial Sequence
  Synthetic construct
AGTYGLGDYSSPGSPGTPGSRC

SEQ ID NO: 178 - protein, 22 aa, Artificial Sequence
  Synthetic construct
AGTYGLGDSSPGSPGTPGSRSC

SEQ ID NO: 179 - protein, 23 aa, Artificial Sequence
  Synthetic construct
AGTYGLGDGYSSPGSPGTPGSRC

SEQ ID NO: 180 - protein, 23 aa, Artificial Sequence
  Synthetic construct
AGTYGLGDYSSPGSPGTPGSRSC

SEQ ID NO: 181 - protein, 23 aa, Artificial Sequence
  Synthetic construct
AGTYGLGDSSPGSPGTPGSRSRC

SEQ ID NO: 182 - protein, 24 aa, Artificial Sequence
  Synthetic construct
AGTYGLGDSGYSSPGSPGTPGSRC

SEQ ID NO: 183 - protein, 24 aa, Artificial Sequence
  Synthetic construct
AGTYGLGDGYSSPGSPGTPGSRSC

SEQ ID NO: 184 - protein, 24 aa, Artificial Sequence
  Synthetic construct
AGTYGLGDYSSPGSPGTPGSRSRC

SEQ ID NO: 185 - protein, 24 aa, Artificial Sequence
  Synthetic construct
AGTYGLGDSSPGSPGTPGSRSRTC

SEQ ID NO: 186 - protein, 25 aa, Artificial Sequence
  Synthetic construct
AGTYGLGDRSGYSSPGSPGTPGSRC

SEQ ID NO: 187 - protein, 25 aa, Artificial Sequence
  Synthetic construct
AGTYGLGDSGYSSPGSPGTPGSRSC

SEQ ID NO: 188 - protein, 25 aa, Artificial Sequence
  Synthetic construct
AGTYGLGDGYSSPGSPGTPGSRSRC

SEQ ID NO: 189 - protein, 25 aa, Artificial Sequence
  Synthetic construct
AGTYGLGDYSSPGSPGTPGSRSRTC

SEQ ID NO: 190 - protein, 25 aa, Artificial Sequence
  Synthetic construct
AGTYGLGDSSPGSPGTPGSRSRTPC

SEQ ID NO: 191 - protein, 26 aa, Artificial Sequence
  Synthetic construct
AGTYGLGDRSGYSSPGSPGTPGSRSC

SEQ ID NO: 192 - protein, 26 aa, Artificial Sequence
  Synthetic construct
AGTYGLGDSGYSSPGSPGTPGSRSRC

SEQ ID NO: 193 - protein, 26 aa, Artificial Sequence
  Synthetic construct
AGTYGLGDGYSSPGSPGTPGSRSRTC

SEQ ID NO: 194 - protein, 26 aa, Artificial Sequence
  Synthetic construct
AGTYGLGDYSSPGSPGTPGSRSRTPC

SEQ ID NO: 195 - protein, 27 aa, Artificial Sequence
  Synthetic construct
AGTYGLGDRSGYSSPGSPGTPGSRSRC

SEQ ID NO: 196 - protein, 27 aa, Artificial Sequence
  Synthetic construct
AGTYGLGDSGYSSPGSPGTPGSRSRTC

SEQ ID NO: 197 - protein, 27 aa, Artificial Sequence
  Synthetic construct
AGTYGLGDGYSSPGSPGTPGSRSRTPC

SEQ ID NO: 198 - protein, 28 aa, Artificial Sequence
  Synthetic construct
AGTYGLGDRSGYSSPGSPGTPGSRSRTC

SEQ ID NO: 199 - protein, 28 aa, Artificial Sequence
  Synthetic construct
AGTYGLGDSGYSSPGSPGTPGSRSRTPC

SEQ ID NO: 200 - protein, 29 aa, Artificial Sequence
  Synthetic construct
AGTYGLGDRSGYSSPGSPGTPGSRSRTPC

SEQ ID NO: 201 - protein, 22 aa, Artificial Sequence
  Synthetic construct
HAGTYGLGDSSPGSPGTPGSRC

SEQ ID NO: 202 - protein, 23 aa, Artificial Sequence
  Synthetic construct
HAGTYGLGDYSSPGSPGTPGSRC

SEQ ID NO: 203 - protein, 23 aa, Artificial Sequence
  Synthetic construct
HAGTYGLGDSSPGSPGTPGSRSC

SEQ ID NO: 204 - protein, 24 aa, Artificial Sequence
  Synthetic construct
HAGTYGLGDGYSSPGSPGTPGSRC

SEQ ID NO: 205 - protein, 24 aa, Artificial Sequence
  Synthetic construct
HAGTYGLGDYSSPGSPGTPGSRSC

SEQ ID NO: 206 - protein, 24 aa, Artificial Sequence
  Synthetic construct
HAGTYGLGDSSPGSPGTPGSRSRC

SEQ ID NO: 207 - protein, 25 aa, Artificial Sequence
  Synthetic construct
HAGTYGLGDSGYSSPGSPGTPGSRC

SEQ ID NO: 208 - protein, 25 aa, Artificial Sequence
  Synthetic construct
HAGTYGLGDGYSSPGSPGTPGSRSC

SEQ ID NO: 209 - protein, 25 aa, Artificial Sequence
  Synthetic construct
HAGTYGLGDYSSPGSPGTPGSRSRC

SEQ ID NO: 210 - protein, 25 aa, Artificial Sequence
  Synthetic construct
HAGTYGLGDSSPGSPGTPGSRSRTC

SEQ ID NO: 211 - protein, 26 aa, Artificial Sequence
  Synthetic construct
HAGTYGLGDRSGYSSPGSPGTPGSRC

SEQ ID NO: 212 - protein, 26 aa, Artificial Sequence
  Synthetic construct
HAGTYGLGDSGYSSPGSPGTPGSRSC

SEQ ID NO: 213 - protein, 26 aa, Artificial Sequence
  Synthetic construct
HAGTYGLGDGYSSPGSPGTPGSRSRC

SEQ ID NO: 214 - protein, 26 aa, Artificial Sequence
  Synthetic construct
HAGTYGLGDYSSPGSPGTPGSRSRTC

SEQ ID NO: 215 - protein, 26 aa, Artificial Sequence
  Synthetic construct
HAGTYGLGDSSPGSPGTPGSRSRTPC

SEQ ID NO: 216 - protein, 27 aa, Artificial Sequence
  Synthetic construct
HAGTYGLGDRSGYSSPGSPGTPGSRSC

SEQ ID NO: 217 - protein, 27 aa, Artificial Sequence
  Synthetic construct
HAGTYGLGDSGYSSPGSPGTPGSRSRC

SEQ ID NO: 218 - protein, 27 aa, Artificial Sequence
  Synthetic construct
HAGTYGLGDGYSSPGSPGTPGSRSRTC

SEQ ID NO: 219 - protein, 27 aa, Artificial Sequence
  Synthetic construct
HAGTYGLGDYSSPGSPGTPGSRSRTPC

SEQ ID NO: 220 - protein, 28 aa, Artificial Sequence
  Synthetic construct
HAGTYGLGDRSGYSSPGSPGTPGSRSRC

SEQ ID NO: 221 - protein, 28 aa, Artificial Sequence
  Synthetic construct
HAGTYGLGDSGYSSPGSPGTPGSRSRTC

SEQ ID NO: 222 - protein, 28 aa, Artificial Sequence
  Synthetic construct
HAGTYGLGDGYSSPGSPGTPGSRSRTPC

SEQ ID NO: 223 - protein, 29 aa, Artificial Sequence
  Synthetic construct
HAGTYGLGDRSGYSSPGSPGTPGSRSRTC

SEQ ID NO: 224 - protein, 29 aa, Artificial Sequence
  Synthetic construct
HAGTYGLGDSGYSSPGSPGTPGSRSRTPC

SEQ ID NO: 225 - protein, 30 aa, Artificial Sequence
  Synthetic construct
HAGTYGLGDRSGYSSPGSPGTPGSRSRTPC

SEQ ID NO: 226 - protein, 13 aa, Artificial Sequence
  Synthetic construct
GTYGLSRTPSLPC

SEQ ID NO: 227 - protein, 14 aa, Artificial Sequence
  Synthetic construct
GTYGLRSRTPSLPC

SEQ ID NO: 228 - protein, 14 aa, Artificial Sequence
  Synthetic construct
GTYGLSRTPSLPTC

SEQ ID NO: 229 - protein, 15 aa, Artificial Sequence
  Synthetic construct
GTYGLSRSRTPSLPC

SEQ ID NO: 230 - protein, 15 aa, Artificial Sequence
  Synthetic construct
GTYGLRSRTPSLPTC

SEQ ID NO: 231 - protein, 15 aa, Artificial Sequence
  Synthetic construct
GTYGLSRTPSLPTPC

SEQ ID NO: 232 - protein, 16 aa, Artificial Sequence
  Synthetic construct
GTYGLSRSRTPSLPTC

SEQ ID NO: 233 - protein, 16 aa, Artificial Sequence
  Synthetic construct
GTYGLRSRTPSLPTPC

SEQ ID NO: 234 - protein, 17 aa, Artificial Sequence
  Synthetic construct
GTYGLSRSRTPSLPTPC

SEQ ID NO: 235 - protein, 14 aa, Artificial Sequence
  Synthetic construct
AGTYGLSRTPSLPC

SEQ ID NO: 236 - protein, 15 aa, Artificial Sequence
  Synthetic construct
AGTYGLRSRTPSLPC

SEQ ID NO: 237 - protein, 15 aa, Artificial Sequence
  Synthetic construct
AGTYGLSRTPSLPTC

SEQ ID NO: 238 - protein, 16 aa, Artificial Sequence
  Synthetic construct
AGTYGLSRSRTPSLPC

SEQ ID NO: 239 - protein, 16 aa, Artificial Sequence
  Synthetic construct
AGTYGLRSRTPSLPTC

SEQ ID NO: 240 - protein, 16 aa, Artificial Sequence
  Synthetic construct
AGTYGLSRTPSLPTPC

SEQ ID NO: 241 - protein, 17 aa, Artificial Sequence
  Synthetic construct
AGTYGLSRSRTPSLPTC

SEQ ID NO: 242 - protein, 17 aa, Artificial Sequence
  Synthetic construct
AGTYGLRSRTPSLPTPC

SEQ ID NO: 243 - protein, 18 aa, Artificial Sequence
  Synthetic construct
AGTYGLSRSRTPSLPTPC

SEQ ID NO: 244 - protein, 14 aa, Artificial Sequence
  Synthetic construct
GTYGLGSRTPSLPC

SEQ ID NO: 245 - protein, 15 aa, Artificial Sequence
  Synthetic construct
GTYGLGRSRTPSLPC

SEQ ID NO: 246 - protein, 15 aa, Artificial Sequence
  Synthetic construct
GTYGLGSRTPSLPTC

SEQ ID NO: 247 - protein, 16 aa, Artificial Sequence
  Synthetic construct
GTYGLGSRSRTPSLPC

SEQ ID NO: 248 - protein, 16 aa, Artificial Sequence
  Synthetic construct
GTYGLGRSRTPSLPTC

SEQ ID NO: 249 - protein, 16 aa, Artificial Sequence
  Synthetic construct
GTYGLGSRTPSLPTPC

SEQ ID NO: 250 - protein, 17 aa, Artificial Sequence
  Synthetic construct
GTYGLGSRSRTPSLPTC

SEQ ID NO: 251 - protein, 17 aa, Artificial Sequence
  Synthetic construct
GTYGLGRSRTPSLPTPC

SEQ ID NO: 252 - protein, 18 aa, Artificial Sequence
  Synthetic construct
GTYGLGSRSRTPSLPTPC

SEQ ID NO: 253 - protein, 15 aa, Artificial Sequence
  Synthetic construct
HAGTYGLSRTPSLPC

SEQ ID NO: 254 - protein, 16 aa, Artificial Sequence
  Synthetic construct
HAGTYGLRSRTPSLPC

SEQ ID NO: 255 - protein, 16 aa, Artificial Sequence
  Synthetic construct
HAGTYGLSRTPSLPTC

SEQ ID NO: 256 - protein, 17 aa, Artificial Sequence
  Synthetic construct
HAGTYGLSRSRTPSLPC

SEQ ID NO: 257 - protein, 17 aa, Artificial Sequence
  Synthetic construct
HAGTYGLRSRTPSLPTC

SEQ ID NO: 258 - protein, 17 aa, Artificial Sequence
  Synthetic construct
HAGTYGLSRTPSLPTPC

SEQ ID NO: 259 - protein, 18 aa, Artificial Sequence
  Synthetic construct
HAGTYGLSRSRTPSLPTC

SEQ ID NO: 260 - protein, 18 aa, Artificial Sequence
  Synthetic construct
HAGTYGLRSRTPSLPTPC

SEQ ID NO: 261 - protein, 19 aa, Artificial Sequence
  Synthetic construct
HAGTYGLSRSRTPSLPTPC

SEQ ID NO: 262 - protein, 15 aa, Artificial Sequence
  Synthetic construct
AGTYGLGSRTPSLPC

SEQ ID NO: 263 - protein, 16 aa, Artificial Sequence
  Synthetic construct
AGTYGLGRSRTPSLPC

SEQ ID NO: 264 - protein, 16 aa, Artificial Sequence
  Synthetic construct
AGTYGLGSRTPSLPTC

SEQ ID NO: 265 - protein, 17 aa, Artificial Sequence
  Synthetic construct
AGTYGLGSRSRTPSLPC

SEQ ID NO: 266 - protein, 17 aa, Artificial Sequence
  Synthetic construct
AGTYGLGRSRTPSLPTC

SEQ ID NO: 267 - protein, 17 aa, Artificial Sequence
  Synthetic construct
AGTYGLGSRTPSLPTPC

SEQ ID NO: 268 - protein, 18 aa, Artificial Sequence
  Synthetic construct
AGTYGLGSRSRTPSLPTC

SEQ ID NO: 269 - protein, 18 aa, Artificial Sequence
  Synthetic construct
AGTYGLGRSRTPSLPTPC

SEQ ID NO: 270 - protein, 19 aa, Artificial Sequence
  Synthetic construct
AGTYGLGSRSRTPSLPTPC

SEQ ID NO: 271 - protein, 15 aa, Artificial Sequence
  Synthetic construct
GTYGLGDSRTPSLPC

SEQ ID NO: 272 - protein, 16 aa, Artificial Sequence
  Synthetic construct
GTYGLGDRSRTPSLPC

SEQ ID NO: 273 - protein, 16 aa, Artificial Sequence
  Synthetic construct
GTYGLGDSRTPSLPTC

SEQ ID NO: 274 - protein, 17 aa, Artificial Sequence
  Synthetic construct
GTYGLGDSRSRTPSLPC

SEQ ID NO: 275 - protein, 17 aa, Artificial Sequence
  Synthetic construct
GTYGLGDRSRTPSLPTC

SEQ ID NO: 276 - protein, 17 aa, Artificial Sequence
  Synthetic construct
GTYGLGDSRTPSLPTPC

SEQ ID NO: 277 - protein, 18 aa, Artificial Sequence
  Synthetic construct
GTYGLGDSRSRTPSLPTC

SEQ ID NO: 278 - protein, 18 aa, Artificial Sequence
  Synthetic construct
GTYGLGDRSRTPSLPTPC

SEQ ID NO: 279 - protein, 19 aa, Artificial Sequence
  Synthetic construct
GTYGLGDSRSRTPSLPTPC

SEQ ID NO: 280 - protein, 16 aa, Artificial Sequence
  Synthetic construct
HAGTYGLGSRTPSLPC

SEQ ID NO: 281 - protein, 17 aa, Artificial Sequence
  Synthetic construct
HAGTYGLGRSRTPSLPC

SEQ ID NO: 282 - protein, 17 aa, Artificial Sequence
  Synthetic construct
HAGTYGLGSRTPSLPTC

SEQ ID NO: 283 - protein, 18 aa, Artificial Sequence
  Synthetic construct
HAGTYGLGSRSRTPSLPC

SEQ ID NO: 284 - protein, 18 aa, Artificial Sequence
  Synthetic construct
HAGTYGLGRSRTPSLPTC

SEQ ID NO: 285 - protein, 18 aa, Artificial Sequence
  Synthetic construct
HAGTYGLGSRTPSLPTPC

SEQ ID NO: 286 - protein, 19 aa, Artificial Sequence
  Synthetic construct
HAGTYGLGSRSRTPSLPTC

SEQ ID NO: 287 - protein, 19 aa, Artificial Sequence
  Synthetic construct
HAGTYGLGRSRTPSLPTPC

SEQ ID NO: 288 - protein, 20 aa, Artificial Sequence
  Synthetic construct
HAGTYGLGSRSRTPSLPTPC

SEQ ID NO: 289 - protein, 16 aa, Artificial Sequence
  Synthetic construct
AGTYGLGDSRTPSLPC

SEQ ID NO: 290 - protein, 17 aa, Artificial Sequence
  Synthetic construct
AGTYGLGDRSRTPSLPC

SEQ ID NO: 291 - protein, 17 aa, Artificial Sequence
  Synthetic construct
AGTYGLGDSRTPSLPTC

SEQ ID NO: 292 - protein, 18 aa, Artificial Sequence
  Synthetic construct
AGTYGLGDSRSRTPSLPC

SEQ ID NO: 293 - protein, 18 aa, Artificial Sequence
  Synthetic construct
AGTYGLGDRSRTPSLPTC

SEQ ID NO: 294 - protein, 18 aa, Artificial Sequence
  Synthetic construct
AGTYGLGDSRTPSLPTPC

SEQ ID NO: 295 - protein, 19 aa, Artificial Sequence
  Synthetic construct
AGTYGLGDSRSRTPSLPTC

SEQ ID NO: 296 - protein, 19 aa, Artificial Sequence
  Synthetic construct
AGTYGLGDRSRTPSLPTPC

SEQ ID NO: 297 - protein, 20 aa, Artificial Sequence
  Synthetic construct
AGTYGLGDSRSRTPSLPTPC

SEQ ID NO: 298 - protein, 17 aa, Artificial Sequence
  Synthetic construct
HAGTYGLGDSRTPSLPC

SEQ ID NO: 299 - protein, 18 aa, Artificial Sequence
  Synthetic construct
HAGTYGLGDRSRTPSLPC

SEQ ID NO: 300 - protein, 18 aa, Artificial Sequence
  Synthetic construct
HAGTYGLGDSRTPSLPTC

SEQ ID NO: 301 - protein, 19 aa, Artificial Sequence
  Synthetic construct
HAGTYGLGDSRSRTPSLPC

SEQ ID NO: 302 - protein, 19 aa, Artificial Sequence
  Synthetic construct
HAGTYGLGDRSRTPSLPTC

SEQ ID NO: 303 - protein, 19 aa, Artificial Sequence
  Synthetic construct
HAGTYGLGDSRTPSLPTPC

SEQ ID NO: 304 - protein, 20 aa, Artificial Sequence
  Synthetic construct
HAGTYGLGDSRSRTPSLPTC

SEQ ID NO: 305 - protein, 20 aa, Artificial Sequence
  Synthetic construct
HAGTYGLGDRSRTPSLPTPC

SEQ ID NO: 306 - protein, 21 aa, Artificial Sequence
  Synthetic construct
HAGTYGLGDSRSRTPSLPTPC

SEQ ID NO: 307 - protein, 15 aa, Artificial Sequence
  Synthetic construct
GTYGLVRTPPKSPSC

SEQ ID NO: 308 - protein, 16 aa, Artificial Sequence
  Synthetic construct
GTYGLVVRTPPKSPSC

SEQ ID NO: 309 - protein, 16 aa, Artificial Sequence
  Synthetic construct
GTYGLVRTPPKSPSSC

SEQ ID NO: 310 - protein, 17 aa, Artificial Sequence
  Synthetic construct
GTYGLAVVRTPPKSPSC

SEQ ID NO: 311 - protein, 17 aa, Artificial Sequence
  Synthetic construct
GTYGLVVRTPPKSPSSC

SEQ ID NO: 312 - protein, 17 aa, Artificial Sequence
  Synthetic construct
GTYGLVRTPPKSPSSAC

SEQ ID NO: 313 - protein, 18 aa, Artificial Sequence
  Synthetic construct
GTYGLAVVRTPPKSPSSC

SEQ ID NO: 314 - protein, 18 aa, Artificial Sequence
  Synthetic construct
GTYGLVVRTPPKSPSSAC

SEQ ID NO: 315 - protein, 19 aa, Artificial Sequence
  Synthetic construct
GTYGLAVVRTPPKSPSSAC

SEQ ID NO: 316 - protein, 16 aa, Artificial Sequence
  Synthetic construct
AGTYGLVRTPPKSPSC

SEQ ID NO: 317 - protein, 17 aa, Artificial Sequence
  Synthetic construct
AGTYGLVVRTPPKSPSC

SEQ ID NO: 318 - protein, 17 aa, Artificial Sequence
  Synthetic construct
AGTYGLVRTPPKSPSSC

SEQ ID NO: 319 - protein, 18 aa, Artificial Sequence
  Synthetic construct
AGTYGLAVVRTPPKSPSC

SEQ ID NO: 320 - protein, 18 aa, Artificial Sequence
  Synthetic construct
AGTYGLVVRTPPKSPSSC

SEQ ID NO: 321 - protein, 18 aa, Artificial Sequence
  Synthetic construct
AGTYGLVRTPPKSPSSAC

SEQ ID NO: 322 - protein, 19 aa, Artificial Sequence
  Synthetic construct
AGTYGLAVVRTPPKSPSSC

SEQ ID NO: 323 - protein, 19 aa, Artificial Sequence
  Synthetic construct
AGTYGLVVRTPPKSPSSAC

SEQ ID NO: 324 - protein, 20 aa, Artificial Sequence
  Synthetic construct
AGTYGLAVVRTPPKSPSSAC

SEQ ID NO: 325 - protein, 16 aa, Artificial Sequence
  Synthetic construct
GTYGLGVRTPPKSPSC

SEQ ID NO: 326 - protein, 17 aa, Artificial Sequence
  Synthetic construct
GTYGLGVVRTPPKSPSC

SEQ ID NO: 327 - protein, 17 aa, Artificial Sequence
  Synthetic construct
GTYGLGVRTPPKSPSSC

SEQ ID NO: 328 - protein, 18 aa, Artificial Sequence
  Synthetic construct
GTYGLGAVVRTPPKSPSC

SEQ ID NO: 329 - protein, 18 aa, Artificial Sequence
  Synthetic construct
GTYGLGVVRTPPKSPSSC

SEQ ID NO: 330 - protein, 18 aa, Artificial Sequence
  Synthetic construct
GTYGLGVRTPPKSPSSAC

SEQ ID NO: 331 - protein, 19 aa, Artificial Sequence
  Synthetic construct
GTYGLGAVVRTPPKSPSSC

SEQ ID NO: 332 - protein, 19 aa, Artificial Sequence
  Synthetic construct
GTYGLGVVRTPPKSPSSAC

SEQ ID NO: 333 - protein, 20 aa, Artificial Sequence
  Synthetic construct
GTYGLGAVVRTPPKSPSSAC

SEQ ID NO: 334 - protein, 17 aa, Artificial Sequence
  Synthetic construct
HAGTYGLVRTPPKSPSC

SEQ ID NO: 335 - protein, 18 aa, Artificial Sequence
  Synthetic construct
HAGTYGLVVRTPPKSPSC

SEQ ID NO: 336 - protein, 18 aa, Artificial Sequence
  Synthetic construct
HAGTYGLVRTPPKSPSSC

SEQ ID NO: 337 - protein, 19 aa, Artificial Sequence
  Synthetic construct
HAGTYGLAVVRTPPKSPSC

SEQ ID NO: 338 - protein, 19 aa, Artificial Sequence
  Synthetic construct
HAGTYGLVVRTPPKSPSSC

SEQ ID NO: 339 - protein, 19 aa, Artificial Sequence
  Synthetic construct
HAGTYGLVRTPPKSPSSAC

SEQ ID NO: 340 - protein, 20 aa, Artificial Sequence
  Synthetic construct
HAGTYGLAVVRTPPKSPSSC

SEQ ID NO: 341 - protein, 20 aa, Artificial Sequence
  Synthetic construct
HAGTYGLVVRTPPKSPSSAC

SEQ ID NO: 342 - protein, 21 aa, Artificial Sequence
  Synthetic construct
HAGTYGLAVVRTPPKSPSSAC

SEQ ID NO: 343 - protein, 17 aa, Artificial Sequence
  Synthetic construct
AGTYGLGVRTPPKSPSC

SEQ ID NO: 344 - protein, 18 aa, Artificial Sequence
  Synthetic construct
AGTYGLGVVRTPPKSPSC

SEQ ID NO: 345 - protein, 18 aa, Artificial Sequence
  Synthetic construct
AGTYGLGVRTPPKSPSSC

SEQ ID NO: 346 - protein, 19 aa, Artificial Sequence
  Synthetic construct
AGTYGLGAVVRTPPKSPSC

SEQ ID NO: 347 - protein, 19 aa, Artificial Sequence
  Synthetic construct
AGTYGLGVVRTPPKSPSSC

SEQ ID NO: 348 - protein, 19 aa, Artificial Sequence
  Synthetic construct
AGTYGLGVRTPPKSPSSAC

SEQ ID NO: 349 - protein, 20 aa, Artificial Sequence
  Synthetic construct
AGTYGLGAVVRTPPKSPSSC

SEQ ID NO: 350 - protein, 20 aa, Artificial Sequence
  Synthetic construct
AGTYGLGVVRTPPKSPSSAC

SEQ ID NO: 351 - protein, 21 aa, Artificial Sequence
  Synthetic construct
AGTYGLGAVVRTPPKSPSSAC

SEQ ID NO: 352 - protein, 17 aa, Artificial Sequence
  Synthetic construct
GTYGLGDVRTPPKSPSC

SEQ ID NO: 353 - protein, 18 aa, Artificial Sequence
  Synthetic construct
GTYGLGDVVRTPPKSPSC

SEQ ID NO: 354 - protein, 18 aa, Artificial Sequence
  Synthetic construct
GTYGLGDVRTPPKSPSSC

SEQ ID NO: 355 - protein, 19 aa, Artificial Sequence
  Synthetic construct
GTYGLGDAVVRTPPKSPSC

SEQ ID NO: 356 - protein, 19 aa, Artificial Sequence
  Synthetic construct
GTYGLGDVVRTPPKSPSSC

SEQ ID NO: 357 - protein, 19 aa, Artificial Sequence
  Synthetic construct
GTYGLGDVRTPPKSPSSAC

SEQ ID NO: 358 - protein, 20 aa, Artificial Sequence
  Synthetic construct
GTYGLGDAVVRTPPKSPSSC

SEQ ID NO: 359 - protein, 20 aa, Artificial Sequence
  Synthetic construct
GTYGLGDVVRTPPKSPSSAC

SEQ ID NO: 360 - protein, 21 aa, Artificial Sequence
  Synthetic construct
GTYGLGDAVVRTPPKSPSSAC

SEQ ID NO: 361 - protein, 18 aa, Artificial Sequence
  Synthetic construct
HAGTYGLGVRTPPKSPSC

SEQ ID NO: 362 - protein, 19 aa, Artificial Sequence
  Synthetic construct
HAGTYGLGVVRTPPKSPSC

SEQ ID NO: 363 - protein, 19 aa, Artificial Sequence
  Synthetic construct
HAGTYGLGVRTPPKSPSSC

SEQ ID NO: 364 - protein, 20 aa, Artificial Sequence
  Synthetic construct
HAGTYGLGAVVRTPPKSPSC

SEQ ID NO: 365 - protein, 20 aa, Artificial Sequence
  Synthetic construct
HAGTYGLGVVRTPPKSPSSC

SEQ ID NO: 366 - protein, 20 aa, Artificial Sequence
  Synthetic construct
HAGTYGLGVRTPPKSPSSAC

SEQ ID NO: 367 - protein, 21 aa, Artificial Sequence
  Synthetic construct
HAGTYGLGAVVRTPPKSPSSC

SEQ ID NO: 368 - protein, 21 aa, Artificial Sequence
  Synthetic construct
HAGTYGLGVVRTPPKSPSSAC

SEQ ID NO: 369 - protein, 22 aa, Artificial Sequence
  Synthetic construct
HAGTYGLGAVVRTPPKSPSSAC

SEQ ID NO: 370 - protein, 18 aa, Artificial Sequence
  Synthetic construct
AGTYGLGDVRTPPKSPSC

SEQ ID NO: 371 - protein, 19 aa, Artificial Sequence
  Synthetic construct
AGTYGLGDVVRTPPKSPSC

SEQ ID NO: 372 - protein, 19 aa, Artificial Sequence
  Synthetic construct
AGTYGLGDVRTPPKSPSSC

SEQ ID NO: 373 - protein, 20 aa, Artificial Sequence
  Synthetic construct
AGTYGLGDAVVRTPPKSPSC

SEQ ID NO: 374 - protein, 20 aa, Artificial Sequence
  Synthetic construct
AGTYGLGDVVRTPPKSPSSC

SEQ ID NO: 375 - protein, 20 aa, Artificial Sequence
  Synthetic construct
AGTYGLGDVRTPPKSPSSAC

SEQ ID NO: 376 - protein, 21 aa, Artificial Sequence
  Synthetic construct
AGTYGLGDAVVRTPPKSPSSC

SEQ ID NO: 377 - protein, 21 aa, Artificial Sequence
  Synthetic construct
AGTYGLGDVVRTPPKSPSSAC

SEQ ID NO: 378 - protein, 22 aa, Artificial Sequence
  Synthetic construct
AGTYGLGDAVVRTPPKSPSSAC

SEQ ID NO: 379 - protein, 19 aa, Artificial Sequence
  Synthetic construct
HAGTYGLGDVRTPPKSPSC

SEQ ID NO: 380 - protein, 20 aa, Artificial Sequence
  Synthetic construct
HAGTYGLGDVVRTPPKSPSC

SEQ ID NO: 381 - protein, 20 aa, Artificial Sequence
  Synthetic construct
HAGTYGLGDVRTPPKSPSSC

SEQ ID NO: 382 - protein, 21 aa, Artificial Sequence
  Synthetic construct
HAGTYGLGDAVVRTPPKSPSC

SEQ ID NO: 383 - protein, 21 aa, Artificial Sequence
  Synthetic construct
HAGTYGLGDVVRTPPKSPSSC

SEQ ID NO: 384 - protein, 21 aa, Artificial Sequence
  Synthetic construct
HAGTYGLGDVRTPPKSPSSAC

SEQ ID NO: 385 - protein, 22 aa, Artificial Sequence
  Synthetic construct
HAGTYGLGDAVVRTPPKSPSSC

SEQ ID NO: 386 - protein, 22 aa, Artificial Sequence
  Synthetic construct
HAGTYGLGDVVRTPPKSPSSAC

SEQ ID NO: 387 - protein, 23 aa, Artificial Sequence
  Synthetic construct
HAGTYGLGDAVVRTPPKSPSSAC

SEQ ID NO: 388 - protein, 20 aa, Artificial Sequence
  Synthetic construct
GTYGLPSSAKSRSKIGSTEC

SEQ ID NO: 389 - protein, 21 aa, Artificial Sequence
  Synthetic construct
GTYGLSPSSAKSRSKIGSTEC

SEQ ID NO: 390 - protein, 21 aa, Artificial Sequence
  Synthetic construct
GTYGLPSSAKSRSKIGSTENC

SEQ ID NO: 391 - protein, 22 aa, Artificial Sequence
  Synthetic construct
GTYGLKSPSSAKSRSKIGSTEC

SEQ ID NO: 392 - protein, 22 aa, Artificial Sequence
  Synthetic construct
GTYGLSPSSAKSRSKIGSTENC

SEQ ID NO: 393 - protein, 22 aa, Artificial Sequence
  Synthetic construct
GTYGLPSSAKSRSKIGSTENLC

SEQ ID NO: 394 - protein, 23 aa, Artificial Sequence
  Synthetic construct
GTYGLKSPSSAKSRSKIGSTENC

SEQ ID NO: 395 - protein, 23 aa, Artificial Sequence
  Synthetic construct
GTYGLSPSSAKSRSKIGSTENLC

SEQ ID NO: 396 - protein, 24 aa, Artificial Sequence
  Synthetic construct
GTYGLKSPSSAKSRSKIGSTENLC

SEQ ID NO: 397 - protein, 21 aa, Artificial Sequence
  Synthetic construct
AGTYGLPSSAKSRSKIGSTEC

SEQ ID NO: 398 - protein, 22 aa, Artificial Sequence
  Synthetic construct
AGTYGLSPSSAKSRSKIGSTEC

SEQ ID NO: 399 - protein, 22 aa, Artificial Sequence
  Synthetic construct
AGTYGLPSSAKSRSKIGSTENC

SEQ ID NO: 400 - protein, 23 aa, Artificial Sequence
  Synthetic construct
AGTYGLKSPSSAKSRSKIGSTEC

SEQ ID NO: 401 - protein, 23 aa, Artificial Sequence
  Synthetic construct
AGTYGLSPSSAKSRSKIGSTENC

SEQ ID NO: 402 - protein, 23 aa, Artificial Sequence
  Synthetic construct
AGTYGLPSSAKSRSKIGSTENLC

SEQ ID NO: 403 - protein, 24 aa, Artificial Sequence
  Synthetic construct
AGTYGLKSPSSAKSRSKIGSTENC

SEQ ID NO: 404 - protein, 24 aa, Artificial Sequence
  Synthetic construct
AGTYGLSPSSAKSRSKIGSTENLC

SEQ ID NO: 405 - protein, 25 aa, Artificial Sequence
  Synthetic construct
AGTYGLKSPSSAKSRSKIGSTENLC

SEQ ID NO: 406 - protein, 21 aa, Artificial Sequence
  Synthetic construct
GTYGLGPSSAKSRSKIGSTEC

SEQ ID NO: 407 - protein, 22 aa, Artificial Sequence
  Synthetic construct
GTYGLGSPSSAKSRSKIGSTEC

SEQ ID NO: 408 - protein, 22 aa, Artificial Sequence
  Synthetic construct
GTYGLGPSSAKSRSKIGSTENC

SEQ ID NO: 409 - protein, 23 aa, Artificial Sequence
  Synthetic construct
GTYGLGKSPSSAKSRSKIGSTEC

SEQ ID NO: 410 - protein, 23 aa, Artificial Sequence
  Synthetic construct
GTYGLGSPSSAKSRSKIGSTENC

SEQ ID NO: 411 - protein, 23 aa, Artificial Sequence
  Synthetic construct
GTYGLGPSSAKSRSKIGSTENLC

SEQ ID NO: 412 - protein, 24 aa, Artificial Sequence
  Synthetic construct
GTYGLGKSPSSAKSRSKIGSTENC

SEQ ID NO: 413 - protein, 24 aa, Artificial Sequence
  Synthetic construct
GTYGLGSPSSAKSRSKIGSTENLC

SEQ ID NO: 414 - protein, 25 aa, Artificial Sequence
  Synthetic construct
GTYGLGKSPSSAKSRSKIGSTENLC

SEQ ID NO: 415 - protein, 22 aa, Artificial Sequence
  Synthetic construct
HAGTYGLPSSAKSRSKIGSTEC

SEQ ID NO: 416 - protein, 23 aa, Artificial Sequence
  Synthetic construct
HAGTYGLSPSSAKSRSKIGSTEC

SEQ ID NO: 417 - protein, 23 aa, Artificial Sequence
  Synthetic construct
HAGTYGLPSSAKSRSKIGSTENC

SEQ ID NO: 418 - protein, 24 aa, Artificial Sequence
  Synthetic construct
HAGTYGLKSPSSAKSRSKIGSTEC

SEQ ID NO: 419 - protein, 24 aa, Artificial Sequence
  Synthetic construct
HAGTYGLSPSSAKSRSKIGSTENC

SEQ ID NO: 420 - protein, 24 aa, Artificial Sequence
  Synthetic construct
HAGTYGLPSSAKSRSKIGSTENLC

SEQ ID NO: 421 - protein, 25 aa, Artificial Sequence
  Synthetic construct
HAGTYGLKSPSSAKSRSKIGSTENC

SEQ ID NO: 422 - protein, 25 aa, Artificial Sequence
  Synthetic construct
HAGTYGLSPSSAKSRSKIGSTENLC

SEQ ID NO: 423 - protein, 26 aa, Artificial Sequence
  Synthetic construct
HAGTYGLKSPSSAKSRSKIGSTENLC

SEQ ID NO: 424 - protein, 22 aa, Artificial Sequence
  Synthetic construct
AGTYGLGPSSAKSRSKIGSTEC

SEQ ID NO: 425 - protein, 23 aa, Artificial Sequence
  Synthetic construct
AGTYGLGSPSSAKSRSKIGSTEC

SEQ ID NO: 426 - protein, 23 aa, Artificial Sequence
  Synthetic construct
AGTYGLGPSSAKSRSKIGSTENC

SEQ ID NO: 427 - protein, 24 aa, Artificial Sequence
  Synthetic construct
AGTYGLGKSPSSAKSRSKIGSTEC

SEQ ID NO: 428 - protein, 24 aa, Artificial Sequence
  Synthetic construct
AGTYGLGSPSSAKSRSKIGSTENC

SEQ ID NO: 429 - protein, 24 aa, Artificial Sequence
  Synthetic construct
AGTYGLGPSSAKSRSKIGSTENLC

SEQ ID NO: 430 - protein, 25 aa, Artificial Sequence
  Synthetic construct
AGTYGLGKSPSSAKSRSKIGSTENC

SEQ ID NO: 431 - protein, 25 aa, Artificial Sequence
  Synthetic construct
AGTYGLGSPSSAKSRSKIGSTENLC

SEQ ID NO: 432 - protein, 26 aa, Artificial Sequence
  Synthetic construct
AGTYGLGKSPSSAKSRSKIGSTENLC

SEQ ID NO: 433 - protein, 22 aa, Artificial Sequence
  Synthetic construct
GTYGLGDPSSAKSRSKIGSTEC

SEQ ID NO: 434 - protein, 23 aa, Artificial Sequence
  Synthetic construct
GTYGLGDSPSSAKSRSKIGSTEC

SEQ ID NO: 435 - protein, 23 aa, Artificial Sequence
  Synthetic construct
GTYGLGDPSSAKSRSKIGSTENC

SEQ ID NO: 436 - protein, 24 aa, Artificial Sequence
  Synthetic construct
GTYGLGDKSPSSAKSRSKIGSTEC

SEQ ID NO: 437 - protein, 24 aa, Artificial Sequence
  Synthetic construct
GTYGLGDSPSSAKSRSKIGSTENC

SEQ ID NO: 438 - protein, 24 aa, Artificial Sequence
  Synthetic construct
GTYGLGDPSSAKSRSKIGSTENLC

SEQ ID NO: 439 - protein, 25 aa, Artificial Sequence
  Synthetic construct
GTYGLGDKSPSSAKSRSKIGSTENC

SEQ ID NO: 440 - protein, 25 aa, Artificial Sequence
  Synthetic construct
GTYGLGDSPSSAKSRSKIGSTENLC

SEQ ID NO: 441 - protein, 26 aa, Artificial Sequence
  Synthetic construct
GTYGLGDKSPSSAKSRSKIGSTENLC

SEQ ID NO: 442 - protein, 23 aa, Artificial Sequence
  Synthetic construct
HAGTYGLGPSSAKSRSKIGSTEC

SEQ ID NO: 443 - protein, 24 aa, Artificial Sequence
  Synthetic construct
HAGTYGLGSPSSAKSRSKIGSTEC

SEQ ID NO: 444 - protein, 24 aa, Artificial Sequence
  Synthetic construct
HAGTYGLGPSSAKSRSKIGSTENC

SEQ ID NO: 445 - protein, 25 aa, Artificial Sequence
  Synthetic construct
HAGTYGLGKSPSSAKSRSKIGSTEC

SEQ ID NO: 446 - protein, 25 aa, Artificial Sequence
  Synthetic construct
HAGTYGLGSPSSAKSRSKIGSTENC

SEQ ID NO: 447 - protein, 25 aa, Artificial Sequence
  Synthetic construct
HAGTYGLGPSSAKSRSKIGSTENLC

SEQ ID NO: 448 - protein, 26 aa, Artificial Sequence
  Synthetic construct
HAGTYGLGKSPSSAKSRSKIGSTENC

SEQ ID NO: 449 - protein, 26 aa, Artificial Sequence
  Synthetic construct
HAGTYGLGSPSSAKSRSKIGSTENLC

SEQ ID NO: 450 - protein, 27 aa, Artificial Sequence
  Synthetic construct
HAGTYGLGKSPSSAKSRSKIGSTENLC

SEQ ID NO: 451 - protein, 23 aa, Artificial Sequence
  Synthetic construct
AGTYGLGDPSSAKSRSKIGSTEC

SEQ ID NO: 452 - protein, 24 aa, Artificial Sequence
  Synthetic construct
AGTYGLGDSPSSAKSRSKIGSTEC

SEQ ID NO: 453 - protein, 24 aa, Artificial Sequence
  Synthetic construct
AGTYGLGDPSSAKSRSKIGSTENC

SEQ ID NO: 454 - protein, 25 aa, Artificial Sequence
  Synthetic construct
AGTYGLGDKSPSSAKSRSKIGSTEC

SEQ ID NO: 455 - protein, 25 aa, Artificial Sequence
  Synthetic construct
AGTYGLGDSPSSAKSRSKIGSTENC

SEQ ID NO: 456 - protein, 25 aa, Artificial Sequence
  Synthetic construct
AGTYGLGDPSSAKSRSKIGSTENLC

SEQ ID NO: 457 - protein, 26 aa, Artificial Sequence
  Synthetic construct
AGTYGLGDKSPSSAKSRSKIGSTENC

SEQ ID NO: 458 - protein, 26 aa, Artificial Sequence
  Synthetic construct
AGTYGLGDSPSSAKSRSKIGSTENLC

SEQ ID NO: 459 - protein, 27 aa, Artificial Sequence
  Synthetic construct
AGTYGLGDKSPSSAKSRSKIGSTENLC

SEQ ID NO: 460 - protein, 24 aa, Artificial Sequence
  Synthetic construct
HAGTYGLGDPSSAKSRSKIGSTEC

SEQ ID NO: 461 - protein, 25 aa, Artificial Sequence
  Synthetic construct
HAGTYGLGDSPSSAKSRSKIGSTEC

SEQ ID NO: 462 - protein, 25 aa, Artificial Sequence
  Synthetic construct
HAGTYGLGDPSSAKSRSKIGSTENC

SEQ ID NO: 463 - protein, 26 aa, Artificial Sequence
  Synthetic construct
HAGTYGLGDKSPSSAKSRSKIGSTEC

SEQ ID NO: 464 - protein, 26 aa, Artificial Sequence
  Synthetic construct
HAGTYGLGDSPSSAKSRSKIGSTENC

SEQ ID NO: 465 - protein, 26 aa, Artificial Sequence
  Synthetic construct
HAGTYGLGDPSSAKSRSKIGSTENLC

SEQ ID NO: 466 - protein, 27 aa, Artificial Sequence
  Synthetic construct
HAGTYGLGDKSPSSAKSRSKIGSTENC

SEQ ID NO: 467 - protein, 27 aa, Artificial Sequence
  Synthetic construct
HAGTYGLGDSPSSAKSRSKIGSTENLC

SEQ ID NO: 468 - protein, 28 aa, Artificial Sequence
  Synthetic construct
HAGTYGLGDKSPSSAKSRSKIGSTENLC

SEQ ID NO: 469 - protein, 18 aa, Artificial Sequence
  Synthetic construct
GTYGLKSPVVSGDTSPRC

SEQ ID NO: 470 - protein, 19 aa, Artificial Sequence
  Synthetic construct
GTYGLYKSPVVSGDTSPRC

SEQ ID NO: 471 - protein, 20 aa, Artificial Sequence
  Synthetic construct
GTYGLVYKSPVVSGDTSPRC

SEQ ID NO: 472 - protein, 20 aa, Artificial Sequence
  Synthetic construct
GTYGLYKSPVVSGDTSPRHC

SEQ ID NO: 473 - protein, 21 aa, Artificial Sequence
  Synthetic construct
GTYGLIVYKSPVVSGDTSPRC

SEQ ID NO: 474 - protein, 21 aa, Artificial Sequence
  Synthetic construct
GTYGLVYKSPVVSGDTSPRHC

SEQ ID NO: 475 - protein, 21 aa, Artificial Sequence
  Synthetic construct
GTYGLYKSPVVSGDTSPRHLC

SEQ ID NO: 476 - protein, 22 aa, Artificial Sequence
  Synthetic construct
GTYGLIVYKSPVVSGDTSPRHC

SEQ ID NO: 477 - protein, 22 aa, Artificial Sequence
  Synthetic construct
GTYGLVYKSPVVSGDTSPRHLC

SEQ ID NO: 478 - protein, 23 aa, Artificial Sequence
  Synthetic construct
GTYGLIVYKSPVVSGDTSPRHLC

SEQ ID NO: 479 - protein, 19 aa, Artificial Sequence
  Synthetic construct
AGTYGLKSPVVSGDTSPRC

SEQ ID NO: 480 - protein, 20 aa, Artificial Sequence
  Synthetic construct
AGTYGLYKSPVVSGDTSPRC

SEQ ID NO: 481 - protein, 21 aa, Artificial Sequence
  Synthetic construct
AGTYGLVYKSPVVSGDTSPRC

SEQ ID NO: 482 - protein, 21 aa, Artificial Sequence
  Synthetic construct
AGTYGLYKSPVVSGDTSPRHC

SEQ ID NO: 483 - protein, 22 aa, Artificial Sequence
  Synthetic construct
AGTYGLIVYKSPVVSGDTSPRC

SEQ ID NO: 484 - protein, 22 aa, Artificial Sequence
  Synthetic construct
AGTYGLVYKSPVVSGDTSPRHC

SEQ ID NO: 485 - protein, 22 aa, Artificial Sequence
  Synthetic construct
AGTYGLYKSPVVSGDTSPRHLC

SEQ ID NO: 486 - protein, 23 aa, Artificial Sequence
  Synthetic construct
AGTYGLIVYKSPVVSGDTSPRHC

SEQ ID NO: 487 - protein, 23 aa, Artificial Sequence
  Synthetic construct
AGTYGLVYKSPVVSGDTSPRHLC

SEQ ID NO: 488 - protein, 24 aa, Artificial Sequence
  Synthetic construct
AGTYGLIVYKSPVVSGDTSPRHLC

SEQ ID NO: 489 - protein, 19 aa, Artificial Sequence
  Synthetic construct
AGTYGLKSPVVSGDTSPRC

SEQ ID NO: 490 - protein, 20 aa, Artificial Sequence
  Synthetic construct
GTYGLGYKSPVVSGDTSPRC

SEQ ID NO: 491 - protein, 21 aa, Artificial Sequence
  Synthetic construct
AGTYGLVYKSPVVSGDTSPRC

SEQ ID NO: 492 - protein, 21 aa, Artificial Sequence
  Synthetic construct
GTYGLGYKSPVVSGDTSPRHC

SEQ ID NO: 493 - protein, 22 aa, Artificial Sequence
  Synthetic construct
GTYGLGIVYKSPVVSGDTSPRC

SEQ ID NO: 494 - protein, 22 aa, Artificial Sequence
  Synthetic construct
GTYGLGVYKSPVVSGDTSPRHC

SEQ ID NO: 495 - protein, 22 aa, Artificial Sequence
  Synthetic construct
GTYGLGYKSPVVSGDTSPRHLC

SEQ ID NO: 496 - protein, 23 aa, Artificial Sequence
  Synthetic construct
GTYGLGIVYKSPVVSGDTSPRHC

SEQ ID NO: 497 - protein, 23 aa, Artificial Sequence
  Synthetic construct
GTYGLGVYKSPVVSGDTSPRHLC

SEQ ID NO: 498 - protein, 24 aa, Artificial Sequence
  Synthetic construct
GTYGLGIVYKSPVVSGDTSPRHLC

SEQ ID NO: 499 - protein, 19 aa, Artificial Sequence
  Synthetic construct
AGTYGLKSPVVSGDTSPRC

SEQ ID NO: 500 - protein, 21 aa, Artificial Sequence
  Synthetic construct
HAGTYGLYKSPVVSGDTSPRC

SEQ ID NO: 501 - protein, 22 aa, Artificial Sequence
  Synthetic construct
HAGTYGLVYKSPVVSGDTSPRC

SEQ ID NO: 502 - protein, 21 aa, Artificial Sequence
  Synthetic construct
AGTYGLYKSPVVSGDTSPRHC

SEQ ID NO: 503 - protein, 23 aa, Artificial Sequence
  Synthetic construct
HAGTYGLIVYKSPVVSGDTSPRC

SEQ ID NO: 504 - protein, 23 aa, Artificial Sequence
  Synthetic construct
HAGTYGLVYKSPVVSGDTSPRHC

SEQ ID NO: 505 - protein, 23 aa, Artificial Sequence
  Synthetic construct
HAGTYGLYKSPVVSGDTSPRHLC

SEQ ID NO: 506 - protein, 24 aa, Artificial Sequence
  Synthetic construct
HAGTYGLIVYKSPVVSGDTSPRHC

SEQ ID NO: 507 - protein, 24 aa, Artificial Sequence
  Synthetic construct
HAGTYGLVYKSPVVSGDTSPRHLC

SEQ ID NO: 508 - protein, 25 aa, Artificial Sequence
  Synthetic construct
HAGTYGLIVYKSPVVSGDTSPRHLC

SEQ ID NO: 509 - protein, 19 aa, Artificial Sequence
  Synthetic construct
AGTYGLKSPVVSGDTSPRC

SEQ ID NO: 510 - protein, 21 aa, Artificial Sequence
  Synthetic construct
AGTYGLGYKSPVVSGDTSPRC

SEQ ID NO: 511 - protein, 21 aa, Artificial Sequence
  Synthetic construct
AGTYGLVYKSPVVSGDTSPRC

SEQ ID NO: 512 - protein, 21 aa, Artificial Sequence
  Synthetic construct
AGTYGLYKSPVVSGDTSPRHC

SEQ ID NO: 513 - protein, 23 aa, Artificial Sequence
  Synthetic construct
AGTYGLGIVYKSPVVSGDTSPRC

SEQ ID NO: 514 - protein, 23 aa, Artificial Sequence
  Synthetic construct
AGTYGLGVYKSPVVSGDTSPRHC

SEQ ID NO: 515 - protein, 23 aa, Artificial Sequence
  Synthetic construct
AGTYGLGYKSPVVSGDTSPRHLC

SEQ ID NO: 516 - protein, 24 aa, Artificial Sequence
  Synthetic construct
AGTYGLGIVYKSPVVSGDTSPRHC

SEQ ID NO: 517 - protein, 24 aa, Artificial Sequence
  Synthetic construct
AGTYGLGVYKSPVVSGDTSPRHLC

SEQ ID NO: 518 - protein, 25 aa, Artificial Sequence
  Synthetic construct
AGTYGLGIVYKSPVVSGDTSPRHLC

SEQ ID NO: 519 - protein, 20 aa, Artificial Sequence
  Synthetic construct
GTYGLGDKSPVVSGDTSPRC

SEQ ID NO: 520 - protein, 21 aa, Artificial Sequence
  Synthetic construct
GTYGLGDYKSPVVSGDTSPRC

SEQ ID NO: 521 - protein, 22 aa, Artificial Sequence
  Synthetic construct
GTYGLGDVYKSPVVSGDTSPRC

SEQ ID NO: 522 - protein, 22 aa, Artificial Sequence
  Synthetic construct
GTYGLGDYKSPVVSGDTSPRHC

SEQ ID NO: 523 - protein, 23 aa, Artificial Sequence
  Synthetic construct
GTYGLGDIVYKSPVVSGDTSPRC

SEQ ID NO: 524 - protein, 23 aa, Artificial Sequence
  Synthetic construct
GTYGLGDVYKSPVVSGDTSPRHC

SEQ ID NO: 525 - protein, 23 aa, Artificial Sequence
  Synthetic construct
GTYGLGDYKSPVVSGDTSPRHLC

SEQ ID NO: 526 - protein, 24 aa, Artificial Sequence
  Synthetic construct
GTYGLGDIVYKSPVVSGDTSPRHC

SEQ ID NO: 527 - protein, 24 aa, Artificial Sequence
  Synthetic construct
GTYGLGDVYKSPVVSGDTSPRHLC

SEQ ID NO: 528 - protein, 25 aa, Artificial Sequence
  Synthetic construct
GTYGLGDIVYKSPVVSGDTSPRHLC

SEQ ID NO: 529 - protein, 21 aa, Artificial Sequence
  Synthetic construct
HAGTYGLGKSPVVSGDTSPRC

SEQ ID NO: 530 - protein, 22 aa, Artificial Sequence
  Synthetic construct
HAGTYGLGYKSPVVSGDTSPRC

SEQ ID NO: 531 - protein, 23 aa, Artificial Sequence
  Synthetic construct
HAGTYGLGVYKSPVVSGDTSPRC

SEQ ID NO: 532 - protein, 23 aa, Artificial Sequence
  Synthetic construct
HAGTYGLGYKSPVVSGDTSPRHC

SEQ ID NO: 533 - protein, 24 aa, Artificial Sequence
  Synthetic construct
HAGTYGLGIVYKSPVVSGDTSPRC

SEQ ID NO: 534 - protein, 24 aa, Artificial Sequence
  Synthetic construct
HAGTYGLGVYKSPVVSGDTSPRHC

SEQ ID NO: 535 - protein, 24 aa, Artificial Sequence
  Synthetic construct
HAGTYGLGYKSPVVSGDTSPRHLC

SEQ ID NO: 536 - protein, 25 aa, Artificial Sequence
  Synthetic construct
HAGTYGLGIVYKSPVVSGDTSPRHC

SEQ ID NO: 537 - protein, 25 aa, Artificial Sequence
  Synthetic construct
HAGTYGLGVYKSPVVSGDTSPRHLC

SEQ ID NO: 538 - protein, 26 aa, Artificial Sequence
  Synthetic construct
HAGTYGLGIVYKSPVVSGDTSPRHLC

SEQ ID NO: 539 - protein, 21 aa, Artificial Sequence
  Synthetic construct
AGTYGLGDKSPVVSGDTSPRC

SEQ ID NO: 540 - protein, 22 aa, Artificial Sequence
  Synthetic construct
AGTYGLGDYKSPVVSGDTSPRC

SEQ ID NO: 541 - protein, 23 aa, Artificial Sequence
  Synthetic construct
AGTYGLGDVYKSPVVSGDTSPRC

SEQ ID NO: 542 - protein, 23 aa, Artificial Sequence
  Synthetic construct
AGTYGLGDYKSPVVSGDTSPRHC

SEQ ID NO: 543 - protein, 24 aa, Artificial Sequence
  Synthetic construct
AGTYGLGDIVYKSPVVSGDTSPRC

SEQ ID NO: 544 - protein, 24 aa, Artificial Sequence
  Synthetic construct
AGTYGLGDVYKSPVVSGDTSPRHC

SEQ ID NO: 545 - protein, 24 aa, Artificial Sequence
  Synthetic construct
AGTYGLGDYKSPVVSGDTSPRHLC

SEQ ID NO: 546 - protein, 25 aa, Artificial Sequence
  Synthetic construct
AGTYGLGDIVYKSPVVSGDTSPRHC

SEQ ID NO: 547 - protein, 25 aa, Artificial Sequence
  Synthetic construct
AGTYGLGDVYKSPVVSGDTSPRHLC

SEQ ID NO: 548 - protein, 26 aa, Artificial Sequence
  Synthetic construct
AGTYGLGDIVYKSPVVSGDTSPRHLC

SEQ ID NO: 549 - protein, 22 aa, Artificial Sequence
  Synthetic construct
HAGTYGLGDKSPVVSGDTSPRC

SEQ ID NO: 550 - protein, 23 aa, Artificial Sequence
  Synthetic construct
HAGTYGLGDYKSPVVSGDTSPRC

SEQ ID NO: 551 - protein, 24 aa, Artificial Sequence
  Synthetic construct
HAGTYGLGDVYKSPVVSGDTSPRC

SEQ ID NO: 552 - protein, 24 aa, Artificial Sequence
  Synthetic construct
HAGTYGLGDYKSPVVSGDTSPRHC

SEQ ID NO: 553 - protein, 25 aa, Artificial Sequence
  Synthetic construct
HAGTYGLGDIVYKSPVVSGDTSPRC

SEQ ID NO: 554 - protein, 25 aa, Artificial Sequence
  Synthetic construct
HAGTYGLGDVYKSPVVSGDTSPRHC

SEQ ID NO: 555 - protein, 25 aa, Artificial Sequence
  Synthetic construct
HAGTYGLGDYKSPVVSGDTSPRHLC

SEQ ID NO: 556 - protein, 26 aa, Artificial Sequence
  Synthetic construct
HAGTYGLGDIVYKSPVVSGDTSPRHC

SEQ ID NO: 557 - protein, 26 aa, Artificial Sequence
  Synthetic construct
HAGTYGLGDVYKSPVVSGDTSPRHLC

SEQ ID NO: 558 - protein, 27 aa, Artificial Sequence
  Synthetic construct
HAGTYGLGDIVYKSPVVSGDTSPRHLC

SEQ ID NO: 559 - protein, 22 aa, Artificial Sequence
  Synthetic construct
SSPGSPGTPGSRVRTPPKSPSC

SEQ ID NO: 560 - protein, 23 aa, Artificial Sequence
  Synthetic construct
SSPGSPGTPGSRVVRTPPKSPSC

SEQ ID NO: 561 - protein, 23 aa, Artificial Sequence
  Synthetic construct
SSPGSPGTPGSRVRTPPKSPSSC

SEQ ID NO: 562 - protein, 24 aa, Artificial Sequence
  Synthetic construct
SSPGSPGTPGSRAVVRTPPKSPSC

SEQ ID NO: 563 - protein, 24 aa, Artificial Sequence
  Synthetic construct
SSPGSPGTPGSRVVRTPPKSPSSC

SEQ ID NO: 564 - protein, 24 aa, Artificial Sequence
  Synthetic construct
SSPGSPGTPGSRVRTPPKSPSSAC

SEQ ID NO: 565 - protein, 25 aa, Artificial Sequence
  Synthetic construct
SSPGSPGTPGSRAVVRTPPKSPSSC

SEQ ID NO: 566 - protein, 25 aa, Artificial Sequence
  Synthetic construct
SSPGSPGTPGSRVVRTPPKSPSSAC

SEQ ID NO: 567 - protein, 26 aa, Artificial Sequence
  Synthetic construct
SSPGSPGTPGSRAVVRTPPKSPSSAC

SEQ ID NO: 568 - protein, 23 aa, Artificial Sequence
  Synthetic construct
YSSPGSPGTPGSRVRTPPKSPSC

SEQ ID NO: 569 - protein, 24 aa, Artificial Sequence
  Synthetic construct
YSSPGSPGTPGSRVVRTPPKSPSC

SEQ ID NO: 570 - protein, 24 aa, Artificial Sequence
  Synthetic construct
YSSPGSPGTPGSRVRTPPKSPSSC

SEQ ID NO: 571 - protein, 25 aa, Artificial Sequence
  Synthetic construct
YSSPGSPGTPGSRAVVRTPPKSPSC

SEQ ID NO: 572 - protein, 25 aa, Artificial Sequence
  Synthetic construct
YSSPGSPGTPGSRVVRTPPKSPSSC

SEQ ID NO: 573 - protein, 25 aa, Artificial Sequence
  Synthetic construct
YSSPGSPGTPGSRVRTPPKSPSSAC

SEQ ID NO: 574 - protein, 26 aa, Artificial Sequence
  Synthetic construct
YSSPGSPGTPGSRAVVRTPPKSPSSC

SEQ ID NO: 575 - protein, 26 aa, Artificial Sequence
  Synthetic construct
YSSPGSPGTPGSRVVRTPPKSPSSAC

SEQ ID NO: 576 - protein, 27 aa, Artificial Sequence
  Synthetic construct
YSSPGSPGTPGSRAVVRTPPKSPSSAC

SEQ ID NO: 577 - protein, 23 aa, Artificial Sequence
  Synthetic construct
SSPGSPGTPGSRSVRTPPKSPSC

SEQ ID NO: 578 - protein, 24 aa, Artificial Sequence
  Synthetic construct
SSPGSPGTPGSRSVVRTPPKSPSC

SEQ ID NO: 579 - protein, 24 aa, Artificial Sequence
  Synthetic construct
SSPGSPGTPGSRSVRTPPKSPSSC

SEQ ID NO: 580 - protein, 25 aa, Artificial Sequence
  Synthetic construct
SSPGSPGTPGSRSAVVRTPPKSPSC

SEQ ID NO: 581 - protein, 25 aa, Artificial Sequence
  Synthetic construct
SSPGSPGTPGSRSVVRTPPKSPSSC

SEQ ID NO: 582 - protein, 25 aa, Artificial Sequence
  Synthetic construct
SSPGSPGTPGSRSVRTPPKSPSSAC

SEQ ID NO: 583 - protein, 26 aa, Artificial Sequence
  Synthetic construct
SSPGSPGTPGSRSAVVRTPPKSPSSC

SEQ ID NO: 584 - protein, 26 aa, Artificial Sequence
  Synthetic construct
SSPGSPGTPGSRSVVRTPPKSPSSAC

SEQ ID NO: 585 - protein, 27 aa, Artificial Sequence
  Synthetic construct
SSPGSPGTPGSRSAVVRTPPKSPSSAC

SEQ ID NO: 586 - protein, 24 aa, Artificial Sequence
  Synthetic construct
GYSSPGSPGTPGSRVRTPPKSPSC

SEQ ID NO: 587 - protein, 25 aa, Artificial Sequence
  Synthetic construct
GYSSPGSPGTPGSRVVRTPPKSPSC

SEQ ID NO: 588 - protein, 25 aa, Artificial Sequence
  Synthetic construct
GYSSPGSPGTPGSRVRTPPKSPSSC

SEQ ID NO: 589 - protein, 26 aa, Artificial Sequence
  Synthetic construct
GYSSPGSPGTPGSRAVVRTPPKSPSC

SEQ ID NO: 590 - protein, 26 aa, Artificial Sequence
  Synthetic construct
GYSSPGSPGTPGSRVVRTPPKSPSSC

SEQ ID NO: 591 - protein, 26 aa, Artificial Sequence
  Synthetic construct
GYSSPGSPGTPGSRVRTPPKSPSSAC

SEQ ID NO: 592 - protein, 27 aa, Artificial Sequence
  Synthetic construct
GYSSPGSPGTPGSRAVVRTPPKSPSSC

SEQ ID NO: 593 - protein, 27 aa, Artificial Sequence
  Synthetic construct
GYSSPGSPGTPGSRVVRTPPKSPSSAC

SEQ ID NO: 594 - protein, 28 aa, Artificial Sequence
  Synthetic construct
GYSSPGSPGTPGSRAVVRTPPKSPSSAC

SEQ ID NO: 595 - protein, 24 aa, Artificial Sequence
  Synthetic construct
YSSPGSPGTPGSRSVRTPPKSPSC

SEQ ID NO: 596 - protein, 25 aa, Artificial Sequence
  Synthetic construct
YSSPGSPGTPGSRSVVRTPPKSPSC

SEQ ID NO: 597 - protein, 25 aa, Artificial Sequence
  Synthetic construct
YSSPGSPGTPGSRSVRTPPKSPSSC

SEQ ID NO: 598 - protein, 26 aa, Artificial Sequence
  Synthetic construct
YSSPGSPGTPGSRSAVVRTPPKSPSC

SEQ ID NO: 599 - protein, 26 aa, Artificial Sequence
  Synthetic construct
YSSPGSPGTPGSRSVVRTPPKSPSSC

SEQ ID NO: 600 - protein, 26 aa, Artificial Sequence
  Synthetic construct
YSSPGSPGTPGSRSVRTPPKSPSSAC

SEQ ID NO: 601 - protein, 27 aa, Artificial Sequence
  Synthetic construct
YSSPGSPGTPGSRSAVVRTPPKSPSSC

SEQ ID NO: 602 - protein, 27 aa, Artificial Sequence
  Synthetic construct
YSSPGSPGTPGSRSVVRTPPKSPSSAC

SEQ ID NO: 603 - protein, 28 aa, Artificial Sequence
  Synthetic construct
YSSPGSPGTPGSRSAVVRTPPKSPSSAC

SEQ ID NO: 604 - protein, 24 aa, Artificial Sequence
  Synthetic construct
SSPGSPGTPGSRSRVRTPPKSPSC

SEQ ID NO: 605 - protein, 25 aa, Artificial Sequence
  Synthetic construct
SSPGSPGTPGSRSRVVRTPPKSPSC

SEQ ID NO: 606 - protein, 25 aa, Artificial Sequence
  Synthetic construct
SSPGSPGTPGSRSRVRTPPKSPSSC

SEQ ID NO: 607 - protein, 26 aa, Artificial Sequence
  Synthetic construct
SSPGSPGTPGSRSRAVVRTPPKSPSC

SEQ ID NO: 608 - protein, 26 aa, Artificial Sequence
  Synthetic construct
SSPGSPGTPGSRSRVVRTPPKSPSSC

SEQ ID NO: 609 - protein, 26 aa, Artificial Sequence
  Synthetic construct
SSPGSPGTPGSRSRVRTPPKSPSSAC

SEQ ID NO: 610 - protein, 27 aa, Artificial Sequence
  Synthetic construct
SSPGSPGTPGSRSRAVVRTPPKSPSSC

SEQ ID NO: 611 - protein, 27 aa, Artificial Sequence
  Synthetic construct
SSPGSPGTPGSRSRVVRTPPKSPSSAC

SEQ ID NO: 612 - protein, 28 aa, Artificial Sequence
  Synthetic construct
SSPGSPGTPGSRSRAVVRTPPKSPSSAC

SEQ ID NO: 613 - protein, 25 aa, Artificial Sequence
  Synthetic construct
SGYSSPGSPGTPGSRVRTPPKSPSC

SEQ ID NO: 614 - protein, 26 aa, Artificial Sequence
  Synthetic construct
SGYSSPGSPGTPGSRVVRTPPKSPSC

SEQ ID NO: 615 - protein, 26 aa, Artificial Sequence
  Synthetic construct
SGYSSPGSPGTPGSRVRTPPKSPSSC

SEQ ID NO: 616 - protein, 27 aa, Artificial Sequence
  Synthetic construct
SGYSSPGSPGTPGSRAVVRTPPKSPSC

SEQ ID NO: 617 - protein, 27 aa, Artificial Sequence
  Synthetic construct
SGYSSPGSPGTPGSRVVRTPPKSPSSC

SEQ ID NO: 618 - protein, 27 aa, Artificial Sequence
  Synthetic construct
SGYSSPGSPGTPGSRVRTPPKSPSSAC

SEQ ID NO: 619 - protein, 28 aa, Artificial Sequence
  Synthetic construct
SGYSSPGSPGTPGSRAVVRTPPKSPSSC

SEQ ID NO: 620 - protein, 28 aa, Artificial Sequence
  Synthetic construct
SGYSSPGSPGTPGSRVVRTPPKSPSSAC

SEQ ID NO: 621 - protein, 29 aa, Artificial Sequence
  Synthetic construct
SGYSSPGSPGTPGSRAVVRTPPKSPSSAC

SEQ ID NO: 622 - protein, 25 aa, Artificial Sequence
  Synthetic construct
GYSSPGSPGTPGSRSVRTPPKSPSC

SEQ ID NO: 623 - protein, 26 aa, Artificial Sequence
  Synthetic construct
GYSSPGSPGTPGSRSVVRTPPKSPSC

SEQ ID NO: 624 - protein, 26 aa, Artificial Sequence
  Synthetic construct
GYSSPGSPGTPGSRSVRTPPKSPSSC

SEQ ID NO: 625 - protein, 27 aa, Artificial Sequence
  Synthetic construct
GYSSPGSPGTPGSRSAVVRTPPKSPSC

SEQ ID NO: 626 - protein, 27 aa, Artificial Sequence
  Synthetic construct
GYSSPGSPGTPGSRSVVRTPPKSPSSC

SEQ ID NO: 627 - protein, 27 aa, Artificial Sequence
  Synthetic construct
GYSSPGSPGTPGSRSVRTPPKSPSSAC

SEQ ID NO: 628 - protein, 28 aa, Artificial Sequence
  Synthetic construct
GYSSPGSPGTPGSRSAVVRTPPKSPSSC

SEQ ID NO: 629 - protein, 28 aa, Artificial Sequence
  Synthetic construct
GYSSPGSPGTPGSRSVVRTPPKSPSSAC

SEQ ID NO: 630 - protein, 29 aa, Artificial Sequence
  Synthetic construct
GYSSPGSPGTPGSRSAVVRTPPKSPSSAC

SEQ ID NO: 631 - protein, 25 aa, Artificial Sequence
  Synthetic construct
YSSPGSPGTPGSRSRVRTPPKSPSC

SEQ ID NO: 632 - protein, 26 aa, Artificial Sequence
  Synthetic construct
YSSPGSPGTPGSRSRVVRTPPKSPSC

SEQ ID NO: 633 - protein, 26 aa, Artificial Sequence
  Synthetic construct
YSSPGSPGTPGSRSRVRTPPKSPSSC

SEQ ID NO: 634 - protein, 27 aa, Artificial Sequence
  Synthetic construct
YSSPGSPGTPGSRSRAVVRTPPKSPSC

SEQ ID NO: 635 - protein, 27 aa, Artificial Sequence
  Synthetic construct
YSSPGSPGTPGSRSRVVRTPPKSPSSC

SEQ ID NO: 636 - protein, 27 aa, Artificial Sequence
  Synthetic construct
YSSPGSPGTPGSRSRVRTPPKSPSSAC

SEQ ID NO: 637 - protein, 28 aa, Artificial Sequence
  Synthetic construct
YSSPGSPGTPGSRSRAVVRTPPKSPSSC

SEQ ID NO: 638 - protein, 28 aa, Artificial Sequence
  Synthetic construct
YSSPGSPGTPGSRSRVVRTPPKSPSSAC

SEQ ID NO: 639 - protein, 29 aa, Artificial Sequence
  Synthetic construct
YSSPGSPGTPGSRSRAVVRTPPKSPSSAC

SEQ ID NO: 640 - protein, 25 aa, Artificial Sequence
  Synthetic construct
SSPGSPGTPGSRSRTVRTPPKSPSC

SEQ ID NO: 641 - protein, 26 aa, Artificial Sequence
  Synthetic construct
SSPGSPGTPGSRSRTVVRTPPKSPSC

SEQ ID NO: 642 - protein, 26 aa, Artificial Sequence
  Synthetic construct
SSPGSPGTPGSRSRTVRTPPKSPSSC

SEQ ID NO: 643 - protein, 27 aa, Artificial Sequence
  Synthetic construct
SSPGSPGTPGSRSRTAVVRTPPKSPSC

SEQ ID NO: 644 - protein, 27 aa, Artificial Sequence
  Synthetic construct
SSPGSPGTPGSRSRTVVRTPPKSPSSC

SEQ ID NO: 645 - protein, 27 aa, Artificial Sequence
  Synthetic construct
SSPGSPGTPGSRSRTVRTPPKSPSSAC

SEQ ID NO: 646 - protein, 28 aa, Artificial Sequence
  Synthetic construct
SSPGSPGTPGSRSRTAVVRTPPKSPSSC

SEQ ID NO: 647 - protein, 28 aa, Artificial Sequence
  Synthetic construct
SSPGSPGTPGSRSRTVVRTPPKSPSSAC

SEQ ID NO: 648 - protein, 29 aa, Artificial Sequence
  Synthetic construct
SSPGSPGTPGSRSRTAVVRTPPKSPSSAC

SEQ ID NO: 649 - protein, 26 aa, Artificial Sequence
  Synthetic construct
RSGYSSPGSPGTPGSRVRTPPKSPSC

SEQ ID NO: 650 - protein, 27 aa, Artificial Sequence
  Synthetic construct
RSGYSSPGSPGTPGSRVVRTPPKSPSC

SEQ ID NO: 651 - protein, 27 aa, Artificial Sequence
  Synthetic construct
RSGYSSPGSPGTPGSRVRTPPKSPSSC

SEQ ID NO: 652 - protein, 28 aa, Artificial Sequence
  Synthetic construct
RSGYSSPGSPGTPGSRAVVRTPPKSPSC

SEQ ID NO: 653 - protein, 28 aa, Artificial Sequence
  Synthetic construct
RSGYSSPGSPGTPGSRVVRTPPKSPSSC

SEQ ID NO: 654 - protein, 28 aa, Artificial Sequence
  Synthetic construct
RSGYSSPGSPGTPGSRVRTPPKSPSSAC

SEQ ID NO: 655 - protein, 29 aa, Artificial Sequence
  Synthetic construct
RSGYSSPGSPGTPGSRAVVRTPPKSPSSC

SEQ ID NO: 656 - protein, 29 aa, Artificial Sequence
  Synthetic construct
RSGYSSPGSPGTPGSRVVRTPPKSPSSAC

SEQ ID NO: 657 - protein, 30 aa, Artificial Sequence
  Synthetic construct
RSGYSSPGSPGTPGSRAVVRTPPKSPSSAC

SEQ ID NO: 658 - protein, 26 aa, Artificial Sequence
  Synthetic construct
SGYSSPGSPGTPGSRSVRTPPKSPSC

SEQ ID NO: 659 - protein, 27 aa, Artificial Sequence
  Synthetic construct
SGYSSPGSPGTPGSRSVVRTPPKSPSC

SEQ ID NO: 660 - protein, 27 aa, Artificial Sequence
  Synthetic construct
SGYSSPGSPGTPGSRSVRTPPKSPSSC

SEQ ID NO: 661 - protein, 28 aa, Artificial Sequence
  Synthetic construct
SGYSSPGSPGTPGSRSAVVRTPPKSPSC

SEQ ID NO: 662 - protein, 28 aa, Artificial Sequence
  Synthetic construct
SGYSSPGSPGTPGSRSVVRTPPKSPSSC

SEQ ID NO: 663 - protein, 28 aa, Artificial Sequence
  Synthetic construct
SGYSSPGSPGTPGSRSVRTPPKSPSSAC

SEQ ID NO: 664 - protein, 29 aa, Artificial Sequence
  Synthetic construct
SGYSSPGSPGTPGSRSAVVRTPPKSPSSC

SEQ ID NO: 665 - protein, 29 aa, Artificial Sequence
  Synthetic construct
SGYSSPGSPGTPGSRSVVRTPPKSPSSAC

SEQ ID NO: 666 - protein, 30 aa, Artificial Sequence
  Synthetic construct
SGYSSPGSPGTPGSRSAVVRTPPKSPSSAC

SEQ ID NO: 667 - protein, 26 aa, Artificial Sequence
  Synthetic construct
GYSSPGSPGTPGSRSRVRTPPKSPSC

SEQ ID NO: 668 - protein, 27 aa, Artificial Sequence
  Synthetic construct
GYSSPGSPGTPGSRSRVVRTPPKSPSC

SEQ ID NO: 669 - protein, 27 aa, Artificial Sequence
  Synthetic construct
GYSSPGSPGTPGSRSRVRTPPKSPSSC

SEQ ID NO: 670 - protein, 28 aa, Artificial Sequence
  Synthetic construct
GYSSPGSPGTPGSRSRAVVRTPPKSPSC

SEQ ID NO: 671 - protein, 28 aa, Artificial Sequence
  Synthetic construct
GYSSPGSPGTPGSRSRVVRTPPKSPSSC

SEQ ID NO: 672 - protein, 28 aa, Artificial Sequence
  Synthetic construct
GYSSPGSPGTPGSRSRVRTPPKSPSSAC

SEQ ID NO: 673 - protein, 29 aa, Artificial Sequence
  Synthetic construct
GYSSPGSPGTPGSRSRAVVRTPPKSPSSC

SEQ ID NO: 674 - protein, 29 aa, Artificial Sequence
  Synthetic construct
GYSSPGSPGTPGSRSRVVRTPPKSPSSAC

SEQ ID NO: 675 - protein, 30 aa, Artificial Sequence
  Synthetic construct
GYSSPGSPGTPGSRSRAVVRTPPKSPSSAC

SEQ ID NO: 676 - protein, 26 aa, Artificial Sequence
  Synthetic construct
YSSPGSPGTPGSRSRTVRTPPKSPSC

SEQ ID NO: 677 - protein, 27 aa, Artificial Sequence
  Synthetic construct
YSSPGSPGTPGSRSRTVVRTPPKSPSC

SEQ ID NO: 678 - protein, 27 aa, Artificial Sequence
  Synthetic construct
YSSPGSPGTPGSRSRTVRTPPKSPSSC

SEQ ID NO: 679 - protein, 28 aa, Artificial Sequence
  Synthetic construct
YSSPGSPGTPGSRSRTAVVRTPPKSPSC

SEQ ID NO: 680 - protein, 28 aa, Artificial Sequence
  Synthetic construct
YSSPGSPGTPGSRSRTVVRTPPKSPSSC

SEQ ID NO: 681 - protein, 28 aa, Artificial Sequence
  Synthetic construct
YSSPGSPGTPGSRSRTVRTPPKSPSSAC

SEQ ID NO: 682 - protein, 29 aa, Artificial Sequence
  Synthetic construct
YSSPGSPGTPGSRSRTAVVRTPPKSPSSC

SEQ ID NO: 683 - protein, 29 aa, Artificial Sequence
  Synthetic construct
YSSPGSPGTPGSRSRTVVRTPPKSPSSAC

SEQ ID NO: 684 - protein, 30 aa, Artificial Sequence
  Synthetic construct
YSSPGSPGTPGSRSRTAVVRTPPKSPSSAC

SEQ ID NO: 685 - protein, 26 aa, Artificial Sequence
  Synthetic construct
SSPGSPGTPGSRSRTPVRTPPKSPSC

SEQ ID NO: 686 - protein, 27 aa, Artificial Sequence
  Synthetic construct
SSPGSPGTPGSRSRTPVVRTPPKSPSC

SEQ ID NO: 687 - protein, 27 aa, Artificial Sequence
  Synthetic construct
SSPGSPGTPGSRSRTPVRTPPKSPSSC

SEQ ID NO: 688 - protein, 28 aa, Artificial Sequence
  Synthetic construct
SSPGSPGTPGSRSRTPAVVRTPPKSPSC

SEQ ID NO: 689 - protein, 28 aa, Artificial Sequence
  Synthetic construct
SSPGSPGTPGSRSRTPVVRTPPKSPSSC

SEQ ID NO: 690 - protein, 28 aa, Artificial Sequence
  Synthetic construct
SSPGSPGTPGSRSRTPVRTPPKSPSSAC

SEQ ID NO: 691 - protein, 29 aa, Artificial Sequence
  Synthetic construct
SSPGSPGTPGSRSRTPAVVRTPPKSPSSC

SEQ ID NO: 692 - protein, 29 aa, Artificial Sequence
  Synthetic construct
SSPGSPGTPGSRSRTPVVRTPPKSPSSAC

SEQ ID NO: 693 - protein, 30 aa, Artificial Sequence
  Synthetic construct
SSPGSPGTPGSRSRTPAVVRTPPKSPSSAC

SEQ ID NO: 694 - protein, 27 aa, Artificial Sequence
  Synthetic construct
RSGYSSPGSPGTPGSRSVRTPPKSPSC

SEQ ID NO: 695 - protein, 28 aa, Artificial Sequence
  Synthetic construct
RSGYSSPGSPGTPGSRSVVRTPPKSPSC

SEQ ID NO: 696 - protein, 28 aa, Artificial Sequence
  Synthetic construct
RSGYSSPGSPGTPGSRSVRTPPKSPSSC

SEQ ID NO: 697 - protein, 29 aa, Artificial Sequence
  Synthetic construct
RSGYSSPGSPGTPGSRSAVVRTPPKSPSC

SEQ ID NO: 698 - protein, 29 aa, Artificial Sequence
  Synthetic construct
RSGYSSPGSPGTPGSRSVVRTPPKSPSSC

SEQ ID NO: 699 - protein, 29 aa, Artificial Sequence
  Synthetic construct
RSGYSSPGSPGTPGSRSVRTPPKSPSSAC

SEQ ID NO: 700 - protein, 30 aa, Artificial Sequence
  Synthetic construct
RSGYSSPGSPGTPGSRSAVVRTPPKSPSSC

SEQ ID NO: 701 - protein, 30 aa, Artificial Sequence
  Synthetic construct
RSGYSSPGSPGTPGSRSVVRTPPKSPSSAC

SEQ ID NO: 702 - protein, 31 aa, Artificial Sequence
  Synthetic construct
RSGYSSPGSPGTPGSRSAVVRTPPKSPSSAC

SEQ ID NO: 703 - protein, 27 aa, Artificial Sequence
  Synthetic construct
SGYSSPGSPGTPGSRSRVRTPPKSPSC

SEQ ID NO: 704 - protein, 28 aa, Artificial Sequence
  Synthetic construct
SGYSSPGSPGTPGSRSRVVRTPPKSPSC

SEQ ID NO: 705 - protein, 28 aa, Artificial Sequence
  Synthetic construct
SGYSSPGSPGTPGSRSRVRTPPKSPSSC

SEQ ID NO: 706 - protein, 29 aa, Artificial Sequence
  Synthetic construct
SGYSSPGSPGTPGSRSRAVVRTPPKSPSC

SEQ ID NO: 707 - protein, 29 aa, Artificial Sequence
  Synthetic construct
SGYSSPGSPGTPGSRSRVVRTPPKSPSSC

SEQ ID NO: 708 - protein, 29 aa, Artificial Sequence
  Synthetic construct
SGYSSPGSPGTPGSRSRVRTPPKSPSSAC

SEQ ID NO: 709 - protein, 30 aa, Artificial Sequence
  Synthetic construct
SGYSSPGSPGTPGSRSRAVVRTPPKSPSSC

SEQ ID NO: 710 - protein, 30 aa, Artificial Sequence
  Synthetic construct
SGYSSPGSPGTPGSRSRVVRTPPKSPSSAC

SEQ ID NO: 711 - protein, 31 aa, Artificial Sequence
  Synthetic construct
SGYSSPGSPGTPGSRSRAVVRTPPKSPSSAC

SEQ ID NO: 712 - protein, 27 aa, Artificial Sequence
  Synthetic construct
GYSSPGSPGTPGSRSRTVRTPPKSPSC

SEQ ID NO: 713 - protein, 28 aa, Artificial Sequence
  Synthetic construct
GYSSPGSPGTPGSRSRTVVRTPPKSPSC

SEQ ID NO: 714 - protein, 28 aa, Artificial Sequence
  Synthetic construct
GYSSPGSPGTPGSRSRTVRTPPKSPSSC

SEQ ID NO: 715 - protein, 29 aa, Artificial Sequence
  Synthetic construct
GYSSPGSPGTPGSRSRTAVVRTPPKSPSC

SEQ ID NO: 716 - protein, 29 aa, Artificial Sequence
  Synthetic construct
GYSSPGSPGTPGSRSRTVVRTPPKSPSSC

SEQ ID NO: 717 - protein, 29 aa, Artificial Sequence
  Synthetic construct
GYSSPGSPGTPGSRSRTVRTPPKSPSSAC

SEQ ID NO: 718 - protein, 30 aa, Artificial Sequence
  Synthetic construct
GYSSPGSPGTPGSRSRTAVVRTPPKSPSSC

SEQ ID NO: 719 - protein, 30 aa, Artificial Sequence
  Synthetic construct
GYSSPGSPGTPGSRSRTVVRTPPKSPSSAC

SEQ ID NO: 720 - protein, 31 aa, Artificial Sequence
  Synthetic construct
GYSSPGSPGTPGSRSRTAVVRTPPKSPSSAC

SEQ ID NO: 721 - protein, 27 aa, Artificial Sequence
  Synthetic construct
YSSPGSPGTPGSRSRTPVRTPPKSPSC

SEQ ID NO: 722 - protein, 28 aa, Artificial Sequence
  Synthetic construct
YSSPGSPGTPGSRSRTPVVRTPPKSPSC

SEQ ID NO: 723 - protein, 28 aa, Artificial Sequence
  Synthetic construct
YSSPGSPGTPGSRSRTPVRTPPKSPSSC

SEQ ID NO: 724 - protein, 29 aa, Artificial Sequence
  Synthetic construct
YSSPGSPGTPGSRSRTPAVVRTPPKSPSC

SEQ ID NO: 725 - protein, 29 aa, Artificial Sequence
  Synthetic construct
YSSPGSPGTPGSRSRTPVVRTPPKSPSSC

SEQ ID NO: 726 - protein, 29 aa, Artificial Sequence
  Synthetic construct
YSSPGSPGTPGSRSRTPVRTPPKSPSSAC

SEQ ID NO: 727 - protein, 30 aa, Artificial Sequence
  Synthetic construct
YSSPGSPGTPGSRSRTPAVVRTPPKSPSSC

SEQ ID NO: 728 - protein, 30 aa, Artificial Sequence
  Synthetic construct
YSSPGSPGTPGSRSRTPVVRTPPKSPSSAC

SEQ ID NO: 729 - protein, 31 aa, Artificial Sequence
  Synthetic construct
YSSPGSPGTPGSRSRTPAVVRTPPKSPSSAC

SEQ ID NO: 730 - protein, 28 aa, Artificial Sequence
  Synthetic construct
RSGYSSPGSPGTPGSRSRVRTPPKSPSC

SEQ ID NO: 731 - protein, 29 aa, Artificial Sequence
  Synthetic construct
RSGYSSPGSPGTPGSRSRVVRTPPKSPSC

SEQ ID NO: 732 - protein, 29 aa, Artificial Sequence
  Synthetic construct
RSGYSSPGSPGTPGSRSRVRTPPKSPSSC

SEQ ID NO: 733 - protein, 30 aa, Artificial Sequence
  Synthetic construct
RSGYSSPGSPGTPGSRSRAVVRTPPKSPSC

SEQ ID NO: 734 - protein, 30 aa, Artificial Sequence
  Synthetic construct
RSGYSSPGSPGTPGSRSRVVRTPPKSPSSC

SEQ ID NO: 735 - protein, 30 aa, Artificial Sequence
  Synthetic construct
RSGYSSPGSPGTPGSRSRVRTPPKSPSSAC

SEQ ID NO: 736 - protein, 31 aa, Artificial Sequence
  Synthetic construct
RSGYSSPGSPGTPGSRSRAVVRTPPKSPSSC

SEQ ID NO: 737 - protein, 31 aa, Artificial Sequence
  Synthetic construct
RSGYSSPGSPGTPGSRSRVVRTPPKSPSSAC

SEQ ID NO: 738 - protein, 28 aa, Artificial Sequence
  Synthetic construct
SGYSSPGSPGTPGSRSRTVRTPPKSPSC

SEQ ID NO: 739 - protein, 29 aa, Artificial Sequence
  Synthetic construct
SGYSSPGSPGTPGSRSRTVVRTPPKSPSC

SEQ ID NO: 740 - protein, 29 aa, Artificial Sequence
  Synthetic construct
SGYSSPGSPGTPGSRSRTVRTPPKSPSSC

SEQ ID NO: 741 - protein, 30 aa, Artificial Sequence
  Synthetic construct
SGYSSPGSPGTPGSRSRTAVVRTPPKSPSC

SEQ ID NO: 742 - protein, 30 aa, Artificial Sequence
  Synthetic construct
SGYSSPGSPGTPGSRSRTVVRTPPKSPSSC

SEQ ID NO: 743 - protein, 30 aa, Artificial Sequence
  Synthetic construct
SGYSSPGSPGTPGSRSRTVRTPPKSPSSAC

SEQ ID NO: 744 - protein, 31 aa, Artificial Sequence
  Synthetic construct
SGYSSPGSPGTPGSRSRTAVVRTPPKSPSSC

SEQ ID NO: 745 - protein, 31 aa, Artificial Sequence
  Synthetic construct
SGYSSPGSPGTPGSRSRTVVRTPPKSPSSAC

SEQ ID NO: 746 - protein, 28 aa, Artificial Sequence
  Synthetic construct
GYSSPGSPGTPGSRSRTPVRTPPKSPSC

SEQ ID NO: 747 - protein, 29 aa, Artificial Sequence
  Synthetic construct
GYSSPGSPGTPGSRSRTPVVRTPPKSPSC

SEQ ID NO: 748 - protein, 29 aa, Artificial Sequence
  Synthetic construct
GYSSPGSPGTPGSRSRTPVRTPPKSPSSC

SEQ ID NO: 749 - protein, 30 aa, Artificial Sequence
  Synthetic construct
GYSSPGSPGTPGSRSRTPAVVRTPPKSPSC

SEQ ID NO: 750 - protein, 30 aa, Artificial Sequence
  Synthetic construct
GYSSPGSPGTPGSRSRTPVVRTPPKSPSSC

SEQ ID NO: 751 - protein, 30 aa, Artificial Sequence
  Synthetic construct
GYSSPGSPGTPGSRSRTPVRTPPKSPSSAC

SEQ ID NO: 752 - protein, 31 aa, Artificial Sequence
  Synthetic construct
GYSSPGSPGTPGSRSRTPAVVRTPPKSPSSC

SEQ ID NO: 753 - protein, 31 aa, Artificial Sequence
  Synthetic construct
GYSSPGSPGTPGSRSRTPVVRTPPKSPSSAC

SEQ ID NO: 754 - protein, 29 aa, Artificial Sequence
  Synthetic construct
RSGYSSPGSPGTPGSRSRTVRTPPKSPSC

SEQ ID NO: 755 - protein, 30 aa, Artificial Sequence
  Synthetic construct
RSGYSSPGSPGTPGSRSRTVVRTPPKSPSC

SEQ ID NO: 756 - protein, 30 aa, Artificial Sequence
  Synthetic construct
RSGYSSPGSPGTPGSRSRTVRTPPKSPSSC

SEQ ID NO: 757 - protein, 31 aa, Artificial Sequence
  Synthetic construct
RSGYSSPGSPGTPGSRSRTAVVRTPPKSPSC

SEQ ID NO: 758 - protein, 31 aa, Artificial Sequence
  Synthetic construct
RSGYSSPGSPGTPGSRSRTVVRTPPKSPSSC

SEQ ID NO: 759 - protein, 31 aa, Artificial Sequence
  Synthetic construct
RSGYSSPGSPGTPGSRSRTVRTPPKSPSSAC

SEQ ID NO: 760 - protein, 32 aa, Artificial Sequence
  Synthetic construct
RSGYSSPGSPGTPGSRSRTAVVRTPPKSPSSC

SEQ ID NO: 761 - protein, 32 aa, Artificial Sequence
  Synthetic construct
RSGYSSPGSPGTPGSRSRTVVRTPPKSPSSAC

SEQ ID NO: 762 - protein, 29 aa, Artificial Sequence
  Synthetic construct
SGYSSPGSPGTPGSRSRTPVRTPPKSPSC

SEQ ID NO: 763 - protein, 30 aa, Artificial Sequence
  Synthetic construct
SGYSSPGSPGTPGSRSRTPVVRTPPKSPSC

SEQ ID NO: 764 - protein, 30 aa, Artificial Sequence
  Synthetic construct
SGYSSPGSPGTPGSRSRTPVRTPPKSPSSC

SEQ ID NO: 765 - protein, 31 aa, Artificial Sequence
  Synthetic construct
SGYSSPGSPGTPGSRSRTPAVVRTPPKSPSC

SEQ ID NO: 766 - protein, 31 aa, Artificial Sequence
  Synthetic construct
SGYSSPGSPGTPGSRSRTPVVRTPPKSPSSC

SEQ ID NO: 767 - protein, 31 aa, Artificial Sequence
  Synthetic construct
SGYSSPGSPGTPGSRSRTPVRTPPKSPSSAC

SEQ ID NO: 768 - protein, 32 aa, Artificial Sequence
  Synthetic construct
SGYSSPGSPGTPGSRSRTPAVVRTPPKSPSSC

SEQ ID NO: 769 - protein, 32 aa, Artificial Sequence
  Synthetic construct
SGYSSPGSPGTPGSRSRTPVVRTPPKSPSSAC

SEQ ID NO: 770 - protein, 30 aa, Artificial Sequence
  Synthetic construct
RSGYSSPGSPGTPGSRSRTPVRTPPKSPSC

SEQ ID NO: 771 - protein, 31 aa, Artificial Sequence
  Synthetic construct
RSGYSSPGSPGTPGSRSRTPVVRTPPKSPSC

SEQ ID NO: 772 - protein, 31 aa, Artificial Sequence
  Synthetic construct
RSGYSSPGSPGTPGSRSRTPVRTPPKSPSSC

SEQ ID NO: 773 - protein, 32 aa, Artificial Sequence
  Synthetic construct
RSGYSSPGSPGTPGSRSRTPAVVRTPPKSPSC

SEQ ID NO: 774 - protein, 32 aa, Artificial Sequence
  Synthetic construct
RSGYSSPGSPGTPGSRSRTPVVRTPPKSPSSC

SEQ ID NO: 775 - protein, 32 aa, Artificial Sequence
  Synthetic construct
RSGYSSPGSPGTPGSRSRTPVRTPPKSPSSAC

SEQ ID NO: 776 - protein, 27 aa, Artificial Sequence
  Synthetic construct
SSPGSPGTPGSRPSSAKSRSKIGSTEC

SEQ ID NO: 777 - protein, 28 aa, Artificial Sequence
  Synthetic construct
SSPGSPGTPGSRSPSSAKSRSKIGSTEC

SEQ ID NO: 778 - protein, 28 aa, Artificial Sequence
  Synthetic construct
SSPGSPGTPGSRPSSAKSRSKIGSTENC

SEQ ID NO: 779 - protein, 29 aa, Artificial Sequence
  Synthetic construct
SSPGSPGTPGSRKSPSSAKSRSKIGSTEC

SEQ ID NO: 780 - protein, 29 aa, Artificial Sequence
  Synthetic construct
SSPGSPGTPGSRSPSSAKSRSKIGSTENC

SEQ ID NO: 781 - protein, 29 aa, Artificial Sequence
  Synthetic construct
SSPGSPGTPGSRPSSAKSRSKIGSTENLC

SEQ ID NO: 782 - protein, 30 aa, Artificial Sequence
  Synthetic construct
SSPGSPGTPGSRKSPSSAKSRSKIGSTENC

SEQ ID NO: 783 - protein, 30 aa, Artificial Sequence
  Synthetic construct
SSPGSPGTPGSRSPSSAKSRSKIGSTENLC

SEQ ID NO: 784 - protein, 31 aa, Artificial Sequence
  Synthetic construct
SSPGSPGTPGSRKSPSSAKSRSKIGSTENLC

SEQ ID NO: 785 - protein, 28 aa, Artificial Sequence
  Synthetic construct
YSSPGSPGTPGSRPSSAKSRSKIGSTEC

SEQ ID NO: 786 - protein, 29 aa, Artificial Sequence
  Synthetic construct
YSSPGSPGTPGSRSPSSAKSRSKIGSTEC

SEQ ID NO: 787 - protein, 29 aa, Artificial Sequence
  Synthetic construct
YSSPGSPGTPGSRPSSAKSRSKIGSTENC

SEQ ID NO: 788 - protein, 30 aa, Artificial Sequence
  Synthetic construct
YSSPGSPGTPGSRKSPSSAKSRSKIGSTEC

SEQ ID NO: 789 - protein, 30 aa, Artificial Sequence
  Synthetic construct
YSSPGSPGTPGSRSPSSAKSRSKIGSTENC

SEQ ID NO: 790 - protein, 30 aa, Artificial Sequence
  Synthetic construct
YSSPGSPGTPGSRPSSAKSRSKIGSTENLC

SEQ ID NO: 791 - protein, 31 aa, Artificial Sequence
  Synthetic construct
YSSPGSPGTPGSRKSPSSAKSRSKIGSTENC

SEQ ID NO: 792 - protein, 31 aa, Artificial Sequence
  Synthetic construct
YSSPGSPGTPGSRSPSSAKSRSKIGSTENLC

SEQ ID NO: 793 - protein, 32 aa, Artificial Sequence
  Synthetic construct
YSSPGSPGTPGSRKSPSSAKSRSKIGSTENLC

SEQ ID NO: 794 - protein, 28 aa, Artificial Sequence
  Synthetic construct
SSPGSPGTPGSRSPSSAKSRSKIGSTEC

SEQ ID NO: 795 - protein, 29 aa, Artificial Sequence
  Synthetic construct
SSPGSPGTPGSRSSPSSAKSRSKIGSTEC

SEQ ID NO: 796 - protein, 29 aa, Artificial Sequence
  Synthetic construct
SSPGSPGTPGSRSPSSAKSRSKIGSTENC

SEQ ID NO: 797 - protein, 30 aa, Artificial Sequence
  Synthetic construct
SSPGSPGTPGSRSKSPSSAKSRSKIGSTEC

SEQ ID NO: 798 - protein, 30 aa, Artificial Sequence
  Synthetic construct
SSPGSPGTPGSRSSPSSAKSRSKIGSTENC

SEQ ID NO: 799 - protein, 30 aa, Artificial Sequence; identical to SEQ ID NO: 783
  Synthetic construct

SEQ ID NO: 800 - protein, 31 aa, Artificial Sequence
  Synthetic construct
SSPGSPGTPGSRSKSPSSAKSRSKIGSTENC

SEQ ID NO: 801 - protein, 31 aa, Artificial Sequence
  Synthetic construct
SSPGSPGTPGSRSSPSSAKSRSKIGSTENLC

SEQ ID NO: 802 - protein, 32 aa, Artificial Sequence
  Synthetic construct
SSPGSPGTPGSRSKSPSSAKSRSKIGSTENLC

SEQ ID NO: 803 - protein, 29 aa, Artificial Sequence
  Synthetic construct
GYSSPGSPGTPGSRPSSAKSRSKIGSTEC

SEQ ID NO: 804 - protein, 30 aa, Artificial Sequence
  Synthetic construct
GYSSPGSPGTPGSRSPSSAKSRSKIGSTEC

SEQ ID NO: 805 - protein, 30 aa, Artificial Sequence
  Synthetic construct
GYSSPGSPGTPGSRPSSAKSRSKIGSTENC

SEQ ID NO: 806 - protein, 31 aa, Artificial Sequence
  Synthetic construct
GYSSPGSPGTPGSRKSPSSAKSRSKIGSTEC

SEQ ID NO: 807 - protein, 31 aa, Artificial Sequence
  Synthetic construct
GYSSPGSPGTPGSRSPSSAKSRSKIGSTENC

SEQ ID NO: 808 - protein, 31 aa, Artificial Sequence
  Synthetic construct
GYSSPGSPGTPGSRPSSAKSRSKIGSTENLC

SEQ ID NO: 809 - protein, 32 aa, Artificial Sequence
  Synthetic construct
GYSSPGSPGTPGSRKSPSSAKSRSKIGSTENC

SEQ ID NO: 810 - protein, 32 aa, Artificial Sequence
  Synthetic construct
GYSSPGSPGTPGSRSPSSAKSRSKIGSTENLC

SEQ ID NO: 811 - protein, 29 aa, Artificial Sequence
  Synthetic construct
YSSPGSPGTPGSRSPSSAKSRSKIGSTEC

SEQ ID NO: 812 - protein, 30 aa, Artificial Sequence
  Synthetic construct
YSSPGSPGTPGSRSSPSSAKSRSKIGSTEC

SEQ ID NO: 813 - protein, 30 aa, Artificial Sequence; identical to SEQ ID NO: 789
  Synthetic construct

SEQ ID NO: 814 - protein, 31 aa, Artificial Sequence
  Synthetic construct
YSSPGSPGTPGSRSKSPSSAKSRSKIGSTEC

SEQ ID NO: 815 - protein, 31 aa, Artificial Sequence
  Synthetic construct
YSSPGSPGTPGSRSSPSSAKSRSKIGSTENC

SEQ ID NO: 816 - protein, 31 aa, Artificial Sequence; identical to SEQ ID NO: 792
  Synthetic construct

SEQ ID NO: 817 - protein, 32 aa, Artificial Sequence
  Synthetic construct
YSSPGSPGTPGSRSKSPSSAKSRSKIGSTENC

SEQ ID NO: 818 - protein, 32 aa, Artificial Sequence
  Synthetic construct
YSSPGSPGTPGSRSSPSSAKSRSKIGSTENLC

SEQ ID NO: 819 - protein, 29 aa, Artificial Sequence
  Synthetic construct
SSPGSPGTPGSRSRPSSAKSRSKIGSTEC

SEQ ID NO: 820 - protein, 30 aa, Artificial Sequence
  Synthetic construct
SSPGSPGTPGSRSRSPSSAKSRSKIGSTEC

SEQ ID NO: 821 - protein, 30 aa, Artificial Sequence
  Synthetic construct
SSPGSPGTPGSRSRPSSAKSRSKIGSTENC

SEQ ID NO: 822 - protein, 31 aa, Artificial Sequence
  Synthetic construct
SSPGSPGTPGSRSRKSPSSAKSRSKIGSTEC

SEQ ID NO: 823 - protein, 31 aa, Artificial Sequence
  Synthetic construct
SSPGSPGTPGSRSRSPSSAKSRSKIGSTENC

SEQ ID NO: 824 - protein, 31 aa, Artificial Sequence
  Synthetic construct
SSPGSPGTPGSRSRPSSAKSRSKIGSTENLC

SEQ ID NO: 825 - protein, 32 aa, Artificial Sequence
  Synthetic construct
SSPGSPGTPGSRSRKSPSSAKSRSKIGSTENC

SEQ ID NO: 826 - protein, 32 aa, Artificial Sequence
  Synthetic construct
SSPGSPGTPGSRSRSPSSAKSRSKIGSTENLC

SEQ ID NO: 827 - protein, 30 aa, Artificial Sequence
  Synthetic construct
SGYSSPGSPGTPGSRPSSAKSRSKIGSTEC

SEQ ID NO: 828 - protein, 31 aa, Artificial Sequence
  Synthetic construct
SGYSSPGSPGTPGSRSPSSAKSRSKIGSTEC

SEQ ID NO: 829 - protein, 31 aa, Artificial Sequence
  Synthetic construct
SGYSSPGSPGTPGSRPSSAKSRSKIGSTENC

SEQ ID NO: 830 - protein, 32 aa, Artificial Sequence
  Synthetic construct
SGYSSPGSPGTPGSRKSPSSAKSRSKIGSTEC

SEQ ID NO: 831 - protein, 32 aa, Artificial Sequence
  Synthetic construct
SGYSSPGSPGTPGSRSPSSAKSRSKIGSTENC

SEQ ID NO: 832 - protein, 32 aa, Artificial Sequence
  Synthetic construct
SGYSSPGSPGTPGSRPSSAKSRSKIGSTENLC

SEQ ID NO: 833 - protein, 30 aa, Artificial Sequence; identical to SEQ ID NO: 804
  Synthetic construct

SEQ ID NO: 834 - protein, 31 aa, Artificial Sequence
  Synthetic construct
GYSSPGSPGTPGSRSSPSSAKSRSKIGSTEC

SEQ ID NO: 835 - protein, 31 aa, Artificial Sequence; identical to SEQ ID NO: 807
  Synthetic construct

SEQ ID NO: 836 - protein, 32 aa, Artificial Sequence
  Synthetic construct
GYSSPGSPGTPGSRSKSPSSAKSRSKIGSTEC

SEQ ID NO: 837 - protein, 32 aa, Artificial Sequence
  Synthetic construct
GYSSPGSPGTPGSRSSPSSAKSRSKIGSTENC

SEQ ID NO: 838 - protein, 32 aa, Artificial Sequence; identical to SEQ ID NO: 810
  Synthetic construct

SEQ ID NO: 839 - protein, 30 aa, Artificial Sequence
  Synthetic construct
YSSPGSPGTPGSRSRPSSAKSRSKIGSTEC

SEQ ID NO: 840 - protein, 31 aa, Artificial Sequence
  Synthetic construct
YSSPGSPGTPGSRSRSPSSAKSRSKIGSTEC

SEQ ID NO: 841 - protein, 31 aa, Artificial Sequence
  Synthetic construct
YSSPGSPGTPGSRSRPSSAKSRSKIGSTENC

SEQ ID NO: 842 - protein, 32 aa, Artificial Sequence
  Synthetic construct
YSSPGSPGTPGSRSRKSPSSAKSRSKIGSTEC

SEQ ID NO: 843 - protein, 32 aa, Artificial Sequence
  Synthetic construct
YSSPGSPGTPGSRSRSPSSAKSRSKIGSTENC

SEQ ID NO: 844 - protein, 32 aa, Artificial Sequence
  Synthetic construct
YSSPGSPGTPGSRSRPSSAKSRSKIGSTENLC

SEQ ID NO: 845 - protein, 30 aa, Artificial Sequence
  Synthetic construct
SSPGSPGTPGSRSRTPSSAKSRSKIGSTEC

SEQ ID NO: 846 - protein, 31 aa, Artificial Sequence
  Synthetic construct
SSPGSPGTPGSRSRTSPSSAKSRSKIGSTEC

SEQ ID NO: 847 - protein, 31 aa, Artificial Sequence
  Synthetic construct
SSPGSPGTPGSRSRTPSSAKSRSKIGSTENC

SEQ ID NO: 848 - protein, 32 aa, Artificial Sequence
  Synthetic construct
SSPGSPGTPGSRSRTKSPSSAKSRSKIGSTEC

SEQ ID NO: 849 - protein, 32 aa, Artificial Sequence
  Synthetic construct
SSPGSPGTPGSRSRTSPSSAKSRSKIGSTENC

SEQ ID NO: 850 - protein, 32 aa, Artificial Sequence
  Synthetic construct
SSPGSPGTPGSRSRTPSSAKSRSKIGSTENLC

SEQ ID NO: 851 - protein, 31 aa, Artificial Sequence
  Synthetic construct
RSGYSSPGSPGTPGSRPSSAKSRSKIGSTEC

SEQ ID NO: 852 - protein, 32 aa, Artificial Sequence
  Synthetic construct
RSGYSSPGSPGTPGSRSPSSAKSRSKIGSTEC

SEQ ID NO: 853 - protein, 32 aa, Artificial Sequence
  Synthetic construct
RSGYSSPGSPGTPGSRPSSAKSRSKIGSTENC

SEQ ID NO: 854 - protein, 31 aa, Artificial Sequence; identical to SEQ ID NO: 828
  Synthetic construct

SEQ ID NO: 855 - protein, 32 aa, Artificial Sequence
  Synthetic construct
SGYSSPGSPGTPGSRSSPSSAKSRSKIGSTEC

SEQ ID NO: 856 - protein, 32 aa, Artificial Sequence; identical to SEQ ID NO: 831
  Synthetic construct

SEQ ID NO: 857 - protein, 31 aa, Artificial Sequence
  Synthetic construct
GYSSPGSPGTPGSRSRPSSAKSRSKIGSTEC

SEQ ID NO: 858 - protein, 32 aa, Artificial Sequence
  Synthetic construct
GYSSPGSPGTPGSRSRSPSSAKSRSKIGSTEC

SEQ ID NO: 859 - protein, 32 aa, Artificial Sequence
  Synthetic construct
GYSSPGSPGTPGSRSRPSSAKSRSKIGSTENC

SEQ ID NO: 860 - protein, 31 aa, Artificial Sequence
  Synthetic construct
YSSPGSPGTPGSRSRTPSSAKSRSKIGSTEC

SEQ ID NO: 861 - protein, 32 aa, Artificial Sequence
  Synthetic construct
YSSPGSPGTPGSRSRTSPSSAKSRSKIGSTEC

SEQ ID NO: 862 - protein, 32 aa, Artificial Sequence
  Synthetic construct
YSSPGSPGTPGSRSRTPSSAKSRSKIGSTENC

SEQ ID NO: 863 - protein, 31 aa, Artificial Sequence
  Synthetic construct
SSPGSPGTPGSRSRTPPSSAKSRSKIGSTEC

SEQ ID NO: 864 - protein, 32 aa, Artificial Sequence
  Synthetic construct
SSPGSPGTPGSRSRTPSPSSAKSRSKIGSTEC

SEQ ID NO: 865 - protein, 32 aa, Artificial Sequence
  Synthetic construct
SSPGSPGTPGSRSRTPPSSAKSRSKIGSTENC

SEQ ID NO: 866 - protein, 25 aa, Artificial Sequence
  Synthetic construct
SSPGSPGTPGSRKSPVVSGDTSPRC

SEQ ID NO: 867 - protein, 26 aa, Artificial Sequence
  Synthetic construct
SSPGSPGTPGSRYKSPVVSGDTSPRC

SEQ ID NO: 868 - protein, 27 aa, Artificial Sequence
  Synthetic construct
SSPGSPGTPGSRVYKSPVVSGDTSPRC

SEQ ID NO: 869 - protein, 27 aa, Artificial Sequence
  Synthetic construct
SSPGSPGTPGSRYKSPVVSGDTSPRHC

SEQ ID NO: 870 - protein, 28 aa, Artificial Sequence
  Synthetic construct
SSPGSPGTPGSRIVYKSPVVSGDTSPRC

SEQ ID NO: 871 - protein, 28 aa, Artificial Sequence
  Synthetic construct
SSPGSPGTPGSRVYKSPVVSGDTSPRHC

SEQ ID NO: 872 - protein, 28 aa, Artificial Sequence
  Synthetic construct
SSPGSPGTPGSRYKSPVVSGDTSPRHLC

SEQ ID NO: 873 - protein, 29 aa, Artificial Sequence
  Synthetic construct
SSPGSPGTPGSRIVYKSPVVSGDTSPRHC

SEQ ID NO: 874 - protein, 29 aa, Artificial Sequence
  Synthetic construct
SSPGSPGTPGSRVYKSPVVSGDTSPRHLC

SEQ ID NO: 875 - protein, 30 aa, Artificial Sequence
  Synthetic construct
SSPGSPGTPGSRIVYKSPVVSGDTSPRHLC

SEQ ID NO: 876 - protein, 26 aa, Artificial Sequence
  Synthetic construct
YSSPGSPGTPGSRKSPVVSGDTSPRC

SEQ ID NO: 877 - protein, 27 aa, Artificial Sequence
  Synthetic construct
YSSPGSPGTPGSRYKSPVVSGDTSPRC

SEQ ID NO: 878 - protein, 28 aa, Artificial Sequence
  Synthetic construct
YSSPGSPGTPGSRVYKSPVVSGDTSPRC

SEQ ID NO: 879 - protein, 28 aa, Artificial Sequence
  Synthetic construct
YSSPGSPGTPGSRYKSPVVSGDTSPRHC

SEQ ID NO: 880 - protein, 29 aa, Artificial Sequence
  Synthetic construct
YSSPGSPGTPGSRIVYKSPVVSGDTSPRC

SEQ ID NO: 881 - protein, 29 aa, Artificial Sequence
  Synthetic construct
YSSPGSPGTPGSRVYKSPVVSGDTSPRHC

SEQ ID NO: 882 - protein, 29 aa, Artificial Sequence
  Synthetic construct
YSSPGSPGTPGSRYKSPVVSGDTSPRHLC

SEQ ID NO: 883 - protein, 30 aa, Artificial Sequence
  Synthetic construct
YSSPGSPGTPGSRIVYKSPVVSGDTSPRHC

SEQ ID NO: 884 - protein, 30 aa, Artificial Sequence
  Synthetic construct
YSSPGSPGTPGSRVYKSPVVSGDTSPRHLC

SEQ ID NO: 885 - protein, 31 aa, Artificial Sequence
  Synthetic construct
YSSPGSPGTPGSRIVYKSPVVSGDTSPRHLC

SEQ ID NO: 886 - protein, 26 aa, Artificial Sequence
  Synthetic construct
SSPGSPGTPGSRSKSPVVSGDTSPRC

SEQ ID NO: 887 - protein, 27 aa, Artificial Sequence
  Synthetic construct
SSPGSPGTPGSRSYKSPVVSGDTSPRC

SEQ ID NO: 888 - protein, 28 aa, Artificial Sequence
  Synthetic construct
SSPGSPGTPGSRSVYKSPVVSGDTSPRC

SEQ ID NO: 889 - protein, 28 aa, Artificial Sequence
  Synthetic construct
SSPGSPGTPGSRSYKSPVVSGDTSPRHC

SEQ ID NO: 890 - protein, 29 aa, Artificial Sequence
  Synthetic construct
SSPGSPGTPGSRSIVYKSPVVSGDTSPRC

SEQ ID NO: 891 - protein, 29 aa, Artificial Sequence
  Synthetic construct
SSPGSPGTPGSRSVYKSPVVSGDTSPRHC

SEQ ID NO: 892 - protein, 29 aa, Artificial Sequence
  Synthetic construct
SSPGSPGTPGSRSYKSPVVSGDTSPRHLC

SEQ ID NO: 893 - protein, 30 aa, Artificial Sequence
  Synthetic construct
SSPGSPGTPGSRSIVYKSPVVSGDTSPRHC

SEQ ID NO: 894 - protein, 30 aa, Artificial Sequence
  Synthetic construct
SSPGSPGTPGSRSVYKSPVVSGDTSPRHLC

SEQ ID NO: 895 - protein, 31 aa, Artificial Sequence
  Synthetic construct
SSPGSPGTPGSRSIVYKSPVVSGDTSPRHLC

SEQ ID NO: 896 - protein, 27 aa, Artificial Sequence
  Synthetic construct
GYSSPGSPGTPGSRKSPVVSGDTSPRC

SEQ ID NO: 897 - protein, 28 aa, Artificial Sequence
  Synthetic construct
GYSSPGSPGTPGSRYKSPVVSGDTSPRC

SEQ ID NO: 898 - protein, 29 aa, Artificial Sequence
  Synthetic construct
GYSSPGSPGTPGSRVYKSPVVSGDTSPRC

SEQ ID NO: 899 - protein, 29 aa, Artificial Sequence
  Synthetic construct
GYSSPGSPGTPGSRYKSPVVSGDTSPRHC

SEQ ID NO: 900 - protein, 30 aa, Artificial Sequence
  Synthetic construct
GYSSPGSPGTPGSRIVYKSPVVSGDTSPRC

SEQ ID NO: 901 - protein, 30 aa, Artificial Sequence
  Synthetic construct
GYSSPGSPGTPGSRVYKSPVVSGDTSPRHC

SEQ ID NO: 902 - protein, 30 aa, Artificial Sequence
  Synthetic construct
GYSSPGSPGTPGSRYKSPVVSGDTSPRHLC

SEQ ID NO: 903 - protein, 31 aa, Artificial Sequence
  Synthetic construct
GYSSPGSPGTPGSRIVYKSPVVSGDTSPRHC

SEQ ID NO: 904 - protein, 31 aa, Artificial Sequence
  Synthetic construct
GYSSPGSPGTPGSRVYKSPVVSGDTSPRHLC

SEQ ID NO: 905 - protein, 32 aa, Artificial Sequence
  Synthetic construct
GYSSPGSPGTPGSRIVYKSPVVSGDTSPRHLC

SEQ ID NO: 906 - protein, 27 aa, Artificial Sequence
  Synthetic construct
YSSPGSPGTPGSRSKSPVVSGDTSPRC

SEQ ID NO: 907 - protein, 28 aa, Artificial Sequence
  Synthetic construct
YSSPGSPGTPGSRSYKSPVVSGDTSPRC

SEQ ID NO: 908 - protein, 29 aa, Artificial Sequence
  Synthetic construct
YSSPGSPGTPGSRSVYKSPVVSGDTSPRC

SEQ ID NO: 909 - protein, 29 aa, Artificial Sequence
  Synthetic construct
YSSPGSPGTPGSRSYKSPVVSGDTSPRHC

SEQ ID NO: 910 - protein, 30 aa, Artificial Sequence
  Synthetic construct
YSSPGSPGTPGSRSIVYKSPVVSGDTSPRC

SEQ ID NO: 911 - protein, 30 aa, Artificial Sequence
  Synthetic construct
YSSPGSPGTPGSRSVYKSPVVSGDTSPRHC

SEQ ID NO: 912 - protein, 30 aa, Artificial Sequence
  Synthetic construct
YSSPGSPGTPGSRSYKSPVVSGDTSPRHLC

SEQ ID NO: 913 - protein, 31 aa, Artificial Sequence
  Synthetic construct
YSSPGSPGTPGSRSIVYKSPVVSGDTSPRHC

SEQ ID NO: 914 - protein, 31 aa, Artificial Sequence
  Synthetic construct
YSSPGSPGTPGSRSVYKSPVVSGDTSPRHLC

SEQ ID NO: 915 - protein, 32 aa, Artificial Sequence
  Synthetic construct
YSSPGSPGTPGSRSIVYKSPVVSGDTSPRHLC

SEQ ID NO: 916 - protein, 27 aa, Artificial Sequence
  Synthetic construct
SSPGSPGTPGSRSRKSPVVSGDTSPRC

SEQ ID NO: 917 - protein, 28 aa, Artificial Sequence
  Synthetic construct
SSPGSPGTPGSRSRYKSPVVSGDTSPRC

SEQ ID NO: 918 - protein, 29 aa, Artificial Sequence
  Synthetic construct
SSPGSPGTPGSRSRVYKSPVVSGDTSPRC

SEQ ID NO: 919 - protein, 29 aa, Artificial Sequence
  Synthetic construct
SSPGSPGTPGSRSRYKSPVVSGDTSPRHC

SEQ ID NO: 920 - protein, 30 aa, Artificial Sequence
  Synthetic construct
SSPGSPGTPGSRSRIVYKSPVVSGDTSPRC

SEQ ID NO: 921 - protein, 30 aa, Artificial Sequence
  Synthetic construct
SSPGSPGTPGSRSRVYKSPVVSGDTSPRHC

SEQ ID NO: 922 - protein, 30 aa, Artificial Sequence
  Synthetic construct
SSPGSPGTPGSRSRYKSPVVSGDTSPRHLC

SEQ ID NO: 923 - protein, 31 aa, Artificial Sequence
  Synthetic construct
SSPGSPGTPGSRSRIVYKSPVVSGDTSPRHC

SEQ ID NO: 924 - protein, 31 aa, Artificial Sequence
  Synthetic construct
SSPGSPGTPGSRSRVYKSPVVSGDTSPRHLC

SEQ ID NO: 925 - protein, 32 aa, Artificial Sequence
  Synthetic construct
SSPGSPGTPGSRSRIVYKSPVVSGDTSPRHLC

SEQ ID NO: 926 - protein, 28 aa, Artificial Sequence
  Synthetic construct
SGYSSPGSPGTPGSRKSPVVSGDTSPRC

SEQ ID NO: 927 - protein, 29 aa, Artificial Sequence
  Synthetic construct
SGYSSPGSPGTPGSRYKSPVVSGDTSPRC

SEQ ID NO: 928 - protein, 30 aa, Artificial Sequence
  Synthetic construct
SGYSSPGSPGTPGSRVYKSPVVSGDTSPRC

SEQ ID NO: 929 - protein, 30 aa, Artificial Sequence
  Synthetic construct
SGYSSPGSPGTPGSRYKSPVVSGDTSPRHC

SEQ ID NO: 930 - protein, 31 aa, Artificial Sequence
  Synthetic construct
SGYSSPGSPGTPGSRIVYKSPVVSGDTSPRC

SEQ ID NO: 931 - protein, 31 aa, Artificial Sequence
  Synthetic construct
SGYSSPGSPGTPGSRVYKSPVVSGDTSPRHC

SEQ ID NO: 932 - protein, 31 aa, Artificial Sequence
  Synthetic construct
SGYSSPGSPGTPGSRYKSPVVSGDTSPRHLC

SEQ ID NO: 933 - protein, 32 aa, Artificial Sequence
  Synthetic construct
SGYSSPGSPGTPGSRIVYKSPVVSGDTSPRHC

SEQ ID NO: 934 - protein, 32 aa, Artificial Sequence
  Synthetic construct
SGYSSPGSPGTPGSRVYKSPVVSGDTSPRHLC

SEQ ID NO: 935 - protein, 28 aa, Artificial Sequence
  Synthetic construct
GYSSPGSPGTPGSRSKSPVVSGDTSPRC

SEQ ID NO: 936 - protein, 29 aa, Artificial Sequence
  Synthetic construct
GYSSPGSPGTPGSRSYKSPVVSGDTSPRC

SEQ ID NO: 937 - protein, 30 aa, Artificial Sequence
  Synthetic construct
GYSSPGSPGTPGSRSVYKSPVVSGDTSPRC

SEQ ID NO: 938 - protein, 30 aa, Artificial Sequence
  Synthetic construct
GYSSPGSPGTPGSRSYKSPVVSGDTSPRHC

SEQ ID NO: 939 - protein, 31 aa, Artificial Sequence
  Synthetic construct
GYSSPGSPGTPGSRSIVYKSPVVSGDTSPRC

SEQ ID NO: 940 - protein, 31 aa, Artificial Sequence
  Synthetic construct
GYSSPGSPGTPGSRSVYKSPVVSGDTSPRHC

SEQ ID NO: 941 - protein, 31 aa, Artificial Sequence
  Synthetic construct
GYSSPGSPGTPGSRSYKSPVVSGDTSPRHLC

SEQ ID NO: 942 - protein, 32 aa, Artificial Sequence
  Synthetic construct
GYSSPGSPGTPGSRSIVYKSPVVSGDTSPRHC

SEQ ID NO: 943 - protein, 32 aa, Artificial Sequence
  Synthetic construct
GYSSPGSPGTPGSRSVYKSPVVSGDTSPRHLC

SEQ ID NO: 944 - protein, 28 aa, Artificial Sequence
  Synthetic construct
YSSPGSPGTPGSRSRKSPVVSGDTSPRC

SEQ ID NO: 945 - protein, 29 aa, Artificial Sequence
  Synthetic construct
YSSPGSPGTPGSRSRYKSPVVSGDTSPRC

SEQ ID NO: 946 - protein, 30 aa, Artificial Sequence
  Synthetic construct
YSSPGSPGTPGSRSRVYKSPVVSGDTSPRC

SEQ ID NO: 947 - protein, 30 aa, Artificial Sequence
  Synthetic construct
YSSPGSPGTPGSRSRYKSPVVSGDTSPRHC

SEQ ID NO: 948 - protein, 31 aa, Artificial Sequence
  Synthetic construct
YSSPGSPGTPGSRSRIVYKSPVVSGDTSPRC

SEQ ID NO: 949 - protein, 31 aa, Artificial Sequence
  Synthetic construct
YSSPGSPGTPGSRSRVYKSPVVSGDTSPRHC

SEQ ID NO: 950 - protein, 31 aa, Artificial Sequence
  Synthetic construct
YSSPGSPGTPGSRSRYKSPVVSGDTSPRHLC

SEQ ID NO: 951 - protein, 32 aa, Artificial Sequence
  Synthetic construct
YSSPGSPGTPGSRSRIVYKSPVVSGDTSPRHC

SEQ ID NO: 952 - protein, 32 aa, Artificial Sequence
  Synthetic construct
YSSPGSPGTPGSRSRVYKSPVVSGDTSPRHLC

SEQ ID NO: 953 - protein, 28 aa, Artificial Sequence
  Synthetic construct
SSPGSPGTPGSRSRTKSPVVSGDTSPRC

SEQ ID NO: 954 - protein, 29 aa, Artificial Sequence
  Synthetic construct
SSPGSPGTPGSRSRTYKSPVVSGDTSPRC

SEQ ID NO: 955 - protein, 30 aa, Artificial Sequence
  Synthetic construct
SSPGSPGTPGSRSRTVYKSPVVSGDTSPRC

SEQ ID NO: 956 - protein, 30 aa, Artificial Sequence
  Synthetic construct
SSPGSPGTPGSRSRTYKSPVVSGDTSPRHC

SEQ ID NO: 957 - protein, 31 aa, Artificial Sequence
  Synthetic construct
SSPGSPGTPGSRSRTIVYKSPVVSGDTSPRC

SEQ ID NO: 958 - protein, 31 aa, Artificial Sequence
  Synthetic construct
SSPGSPGTPGSRSRTVYKSPVVSGDTSPRHC

SEQ ID NO: 959 - protein, 31 aa, Artificial Sequence
  Synthetic construct
SSPGSPGTPGSRSRTYKSPVVSGDTSPRHLC

SEQ ID NO: 960 - protein, 32 aa, Artificial Sequence
  Synthetic construct
SSPGSPGTPGSRSRTIVYKSPVVSGDTSPRHC

SEQ ID NO: 961 - protein, 32 aa, Artificial Sequence
  Synthetic construct
SSPGSPGTPGSRSRTVYKSPVVSGDTSPRHLC

SEQ ID NO: 962 - protein, 29 aa, Artificial Sequence
  Synthetic construct
RSGYSSPGSPGTPGSRKSPVVSGDTSPRC

SEQ ID NO: 963 - protein, 30 aa, Artificial Sequence
  Synthetic construct
RSGYSSPGSPGTPGSRYKSPVVSGDTSPRC

SEQ ID NO: 964 - protein, 31 aa, Artificial Sequence
  Synthetic construct
RSGYSSPGSPGTPGSRVYKSPVVSGDTSPRC

SEQ ID NO: 965 - protein, 31 aa, Artificial Sequence
  Synthetic construct
RSGYSSPGSPGTPGSRYKSPVVSGDTSPRHC

SEQ ID NO: 966 - protein, 32 aa, Artificial Sequence
  Synthetic construct
RSGYSSPGSPGTPGSRIVYKSPVVSGDTSPRC

SEQ ID NO: 967 - protein, 32 aa, Artificial Sequence
  Synthetic construct
RSGYSSPGSPGTPGSRVYKSPVVSGDTSPRHC

SEQ ID NO: 968 - protein, 32 aa, Artificial Sequence
  Synthetic construct
RSGYSSPGSPGTPGSRYKSPVVSGDTSPRHLC

SEQ ID NO: 969 - protein, 29 aa, Artificial Sequence
  Synthetic construct
SGYSSPGSPGTPGSRSKSPVVSGDTSPRC

SEQ ID NO: 970 - protein, 30 aa, Artificial Sequence
  Synthetic construct
SGYSSPGSPGTPGSRSYKSPVVSGDTSPRC

SEQ ID NO: 971 - protein, 31 aa, Artificial Sequence
  Synthetic construct
SGYSSPGSPGTPGSRSVYKSPVVSGDTSPRC

SEQ ID NO: 972 - protein, 31 aa, Artificial Sequence
  Synthetic construct
SGYSSPGSPGTPGSRSYKSPVVSGDTSPRHC

SEQ ID NO: 973 - protein, 32 aa, Artificial Sequence
  Synthetic construct
SGYSSPGSPGTPGSRSIVYKSPVVSGDTSPRC

SEQ ID NO: 974 - protein, 32 aa, Artificial Sequence
  Synthetic construct
SGYSSPGSPGTPGSRSVYKSPVVSGDTSPRHC

SEQ ID NO: 975 - protein, 32 aa, Artificial Sequence
  Synthetic construct
SGYSSPGSPGTPGSRSYKSPVVSGDTSPRHLC

SEQ ID NO: 976 - protein, 29 aa, Artificial Sequence
  Synthetic construct
GYSSPGSPGTPGSRSRKSPVVSGDTSPRC

SEQ ID NO: 977 - protein, 30 aa, Artificial Sequence
  Synthetic construct
GYSSPGSPGTPGSRSRYKSPVVSGDTSPRC

SEQ ID NO: 978 - protein, 31 aa, Artificial Sequence
  Synthetic construct
GYSSPGSPGTPGSRSRVYKSPVVSGDTSPRC

SEQ ID NO: 979 - protein, 31 aa, Artificial Sequence
  Synthetic construct
GYSSPGSPGTPGSRSRYKSPVVSGDTSPRHC

SEQ ID NO: 980 - protein, 32 aa, Artificial Sequence
  Synthetic construct
GYSSPGSPGTPGSRSRIVYKSPVVSGDTSPRC

SEQ ID NO: 981 - protein, 32 aa, Artificial Sequence
  Synthetic construct
GYSSPGSPGTPGSRSRVYKSPVVSGDTSPRHC

SEQ ID NO: 982 - protein, 32 aa, Artificial Sequence
  Synthetic construct
GYSSPGSPGTPGSRSRYKSPVVSGDTSPRHLC

SEQ ID NO: 983 - protein, 29 aa, Artificial Sequence
  Synthetic construct
YSSPGSPGTPGSRSRTKSPVVSGDTSPRC

SEQ ID NO: 984 - protein, 30 aa, Artificial Sequence
  Synthetic construct
YSSPGSPGTPGSRSRTYKSPVVSGDTSPRC

SEQ ID NO: 985 - protein, 31 aa, Artificial Sequence
  Synthetic construct
YSSPGSPGTPGSRSRTVYKSPVVSGDTSPRC

SEQ ID NO: 986 - protein, 31 aa, Artificial Sequence
  Synthetic construct
YSSPGSPGTPGSRSRTYKSPVVSGDTSPRHC

SEQ ID NO: 987 - protein, 32 aa, Artificial Sequence
  Synthetic construct
YSSPGSPGTPGSRSRTIVYKSPVVSGDTSPRC

SEQ ID NO: 988 - protein, 32 aa, Artificial Sequence
  Synthetic construct
YSSPGSPGTPGSRSRTVYKSPVVSGDTSPRHC

SEQ ID NO: 989 - protein, 32 aa, Artificial Sequence
  Synthetic construct
YSSPGSPGTPGSRSRTYKSPVVSGDTSPRHLC

SEQ ID NO: 990 - protein, 29 aa, Artificial Sequence
  Synthetic construct
SSPGSPGTPGSRSRTPKSPVVSGDTSPRC

SEQ ID NO: 991 - protein, 30 aa, Artificial Sequence
  Synthetic construct
SSPGSPGTPGSRSRTPYKSPVVSGDTSPRC

SEQ ID NO: 992 - protein, 31 aa, Artificial Sequence
  Synthetic construct
SSPGSPGTPGSRSRTPVYKSPVVSGDTSPRC

SEQ ID NO: 993 - protein, 31 aa, Artificial Sequence
  Synthetic construct
SSPGSPGTPGSRSRTPYKSPVVSGDTSPRHC

SEQ ID NO: 994 - protein, 32 aa, Artificial Sequence
  Synthetic construct
SSPGSPGTPGSRSRTPIVYKSPVVSGDTSPRC

SEQ ID NO: 995 - protein, 32 aa, Artificial Sequence
  Synthetic construct
SSPGSPGTPGSRSRTPVYKSPVVSGDTSPRHC

SEQ ID NO: 996 - protein, 32 aa, Artificial Sequence
  Synthetic construct
SSPGSPGTPGSRSRTPYKSPVVSGDTSPRHLC

SEQ ID NO: 997 - protein, 30 aa, Artificial Sequence
  Synthetic construct
RSGYSSPGSPGTPGSRSKSPVVSGDTSPRC

SEQ ID NO: 998 - protein, 31 aa, Artificial Sequence
  Synthetic construct
RSGYSSPGSPGTPGSRSYKSPVVSGDTSPRC

SEQ ID NO: 999 - protein, 32 aa, Artificial Sequence
  Synthetic construct
RSGYSSPGSPGTPGSRSVYKSPVVSGDTSPRC

SEQ ID NO: 1000 - protein, 32 aa, Artificial Sequence
  Synthetic construct
RSGYSSPGSPGTPGSRSYKSPVVSGDTSPRHC

SEQ ID NO: 1001 - protein, 30 aa, Artificial Sequence
  Synthetic construct
SGYSSPGSPGTPGSRSRKSPVVSGDTSPRC

SEQ ID NO: 1002 - protein, 31 aa, Artificial Sequence
  Synthetic construct
SGYSSPGSPGTPGSRSRYKSPVVSGDTSPRC

SEQ ID NO: 1003 - protein, 32 aa, Artificial Sequence
  Synthetic construct
SGYSSPGSPGTPGSRSRVYKSPVVSGDTSPRC

SEQ ID NO: 1004 - protein, 32 aa, Artificial Sequence
  Synthetic construct
SGYSSPGSPGTPGSRSRYKSPVVSGDTSPRHC

SEQ ID NO: 1005 - protein, 30 aa, Artificial Sequence
  Synthetic construct
GYSSPGSPGTPGSRSRTKSPVVSGDTSPRC

SEQ ID NO: 1006 - protein, 31 aa, Artificial Sequence
  Synthetic construct
GYSSPGSPGTPGSRSRTYKSPVVSGDTSPRC

SEQ ID NO: 1007 - protein, 32 aa, Artificial Sequence
  Synthetic construct
GYSSPGSPGTPGSRSRTVYKSPVVSGDTSPRC

SEQ ID NO: 1008 - protein, 32 aa, Artificial Sequence
  Synthetic construct
GYSSPGSPGTPGSRSRTYKSPVVSGDTSPRHC

SEQ ID NO: 1009 - protein, 30 aa, Artificial Sequence
  Synthetic construct
YSSPGSPGTPGSRSRTPKSPVVSGDTSPRC

SEQ ID NO: 1010 - protein, 31 aa, Artificial Sequence
  Synthetic construct
YSSPGSPGTPGSRSRTPYKSPVVSGDTSPRC

SEQ ID NO: 1011 - protein, 32 aa, Artificial Sequence
  Synthetic construct
YSSPGSPGTPGSRSRTPVYKSPVVSGDTSPRC

SEQ ID NO: 1012 - protein, 32 aa, Artificial Sequence
  Synthetic construct
YSSPGSPGTPGSRSRTPYKSPVVSGDTSPRHC

SEQ ID NO: 1013 - protein, 31 aa, Artificial Sequence
  Synthetic construct
RSGYSSPGSPGTPGSRSRKSPVVSGDTSPRC

SEQ ID NO: 1014 - protein, 32 aa, Artificial Sequence
  Synthetic construct
RSGYSSPGSPGTPGSRSRYKSPVVSGDTSPRC

SEQ ID NO: 1015 - protein, 31 aa, Artificial Sequence
  Synthetic construct
SGYSSPGSPGTPGSRSRTKSPVVSGDTSPRC

SEQ ID NO: 1016 - protein, 32 aa, Artificial Sequence
  Synthetic construct
SGYSSPGSPGTPGSRSRTYKSPVVSGDTSPRC

SEQ ID NO: 1017 - protein, 31 aa, Artificial Sequence
  Synthetic construct
GYSSPGSPGTPGSRSRTPKSPVVSGDTSPRC

SEQ ID NO: 1018 - protein, 32 aa, Artificial Sequence
  Synthetic construct
GYSSPGSPGTPGSRSRTPYKSPVVSGDTSPRC

SEQ ID NO: 1019 - protein, 32 aa, Artificial Sequence
  Synthetic construct
RSGYSSPGSPGTPGSRSRTKSPVVSGDTSPRC

SEQ ID NO: 1020 - protein, 32 aa, Artificial Sequence
  Synthetic construct
SGYSSPGSPGTPGSRSRTPKSPVVSGDTSPRC

SEQ ID NO: 1021 - protein, 17 aa, Artificial Sequence
  Synthetic construct
SRTPSLPVRTPPKSPSC

SEQ ID NO: 1022 - protein, 18 aa, Artificial Sequence
  Synthetic construct
SRTPSLPVVRTPPKSPSC

SEQ ID NO: 1023 - protein, 18 aa, Artificial Sequence
  Synthetic construct
SRTPSLPVRTPPKSPSSC

SEQ ID NO: 1024 - protein, 19 aa, Artificial Sequence
  Synthetic construct
SRTPSLPAVVRTPPKSPSC

SEQ ID NO: 1025 - protein, 19 aa, Artificial Sequence
  Synthetic construct
SRTPSLPVVRTPPKSPSSC

SEQ ID NO: 1026 - protein, 19 aa, Artificial Sequence
  Synthetic construct
SRTPSLPVRTPPKSPSSAC

SEQ ID NO: 1027 - protein, 20 aa, Artificial Sequence
  Synthetic construct
SRTPSLPAVVRTPPKSPSSC

SEQ ID NO: 1028 - protein, 20 aa, Artificial Sequence
  Synthetic construct
SRTPSLPVVRTPPKSPSSAC

SEQ ID NO: 1029 - protein, 21 aa, Artificial Sequence
  Synthetic construct
SRTPSLPAVVRTPPKSPSSAC

SEQ ID NO: 1030 - protein, 18 aa, Artificial Sequence
  Synthetic construct
RSRTPSLPVRTPPKSPSC

SEQ ID NO: 1031 - protein, 19 aa, Artificial Sequence
  Synthetic construct
RSRTPSLPVVRTPPKSPSC

SEQ ID NO: 1032 - protein, 19 aa, Artificial Sequence
  Synthetic construct
RSRTPSLPVRTPPKSPSSC

SEQ ID NO: 1033 - protein, 20 aa, Artificial Sequence
  Synthetic construct
RSRTPSLPAVVRTPPKSPSC

SEQ ID NO: 1034 - protein, 20 aa, Artificial Sequence
  Synthetic construct
RSRTPSLPVVRTPPKSPSSC

SEQ ID NO: 1035 - protein, 20 aa, Artificial Sequence
  Synthetic construct
RSRTPSLPVRTPPKSPSSAC

SEQ ID NO: 1036 - protein, 21 aa, Artificial Sequence
  Synthetic construct
RSRTPSLPAVVRTPPKSPSSC

SEQ ID NO: 1037 - protein, 21 aa, Artificial Sequence
  Synthetic construct
RSRTPSLPVVRTPPKSPSSAC

SEQ ID NO: 1038 - protein, 22 aa, Artificial Sequence
  Synthetic construct
RSRTPSLPAVVRTPPKSPSSAC

SEQ ID NO: 1039 - protein, 18 aa, Artificial Sequence
  Synthetic construct
SRTPSLPTVRTPPKSPSC

SEQ ID NO: 1040 - protein, 19 aa, Artificial Sequence
  Synthetic construct
SRTPSLPTVVRTPPKSPSC

SEQ ID NO: 1041 - protein, 19 aa, Artificial Sequence
  Synthetic construct
SRTPSLPTVRTPPKSPSSC

SEQ ID NO: 1042 - protein, 20 aa, Artificial Sequence
  Synthetic construct
SRTPSLPTAVVRTPPKSPSC

SEQ ID NO: 1043 - protein, 20 aa, Artificial Sequence
  Synthetic construct
SRTPSLPTVVRTPPKSPSSC

SEQ ID NO: 1044 - protein, 20 aa, Artificial Sequence
  Synthetic construct
SRTPSLPTVRTPPKSPSSAC

SEQ ID NO: 1045 - protein, 21 aa, Artificial Sequence
  Synthetic construct
SRTPSLPTAVVRTPPKSPSSC

SEQ ID NO: 1046 - protein, 21 aa, Artificial Sequence
  Synthetic construct
SRTPSLPTVVRTPPKSPSSAC

SEQ ID NO: 1047 - protein, 22 aa, Artificial Sequence
  Synthetic construct
SRTPSLPTAVVRTPPKSPSSAC

SEQ ID NO: 1048 - protein, 19 aa, Artificial Sequence
  Synthetic construct
SRSRTPSLPVRTPPKSPSC

SEQ ID NO: 1049 - protein, 20 aa, Artificial Sequence
  Synthetic construct
SRSRTPSLPVVRTPPKSPSC

SEQ ID NO: 1050 - protein, 20 aa, Artificial Sequence
  Synthetic construct
SRSRTPSLPVRTPPKSPSSC

SEQ ID NO: 1051 - protein, 21 aa, Artificial Sequence
  Synthetic construct
SRSRTPSLPAVVRTPPKSPSC

SEQ ID NO: 1052 - protein, 21 aa, Artificial Sequence
  Synthetic construct
SRSRTPSLPVVRTPPKSPSSC

SEQ ID NO: 1053 - protein, 21 aa, Artificial Sequence
  Synthetic construct
SRSRTPSLPVRTPPKSPSSAC

SEQ ID NO: 1054 - protein, 22 aa, Artificial Sequence
  Synthetic construct
SRSRTPSLPAVVRTPPKSPSSC

SEQ ID NO: 1055 - protein, 22 aa, Artificial Sequence
  Synthetic construct
SRSRTPSLPVVRTPPKSPSSAC

SEQ ID NO: 1056 - protein, 23 aa, Artificial Sequence
  Synthetic construct
SRSRTPSLPAVVRTPPKSPSSAC

SEQ ID NO: 1057 - protein, 19 aa, Artificial Sequence
  Synthetic construct
RSRTPSLPTVRTPPKSPSC

SEQ ID NO: 1058 - protein, 20 aa, Artificial Sequence
  Synthetic construct
RSRTPSLPTVVRTPPKSPSC

SEQ ID NO: 1059 - protein, 20 aa, Artificial Sequence
  Synthetic construct
RSRTPSLPTVRTPPKSPSSC

SEQ ID NO: 1060 - protein, 21 aa, Artificial Sequence
  Synthetic construct
RSRTPSLPTAVVRTPPKSPSC

SEQ ID NO: 1061 - protein, 21 aa, Artificial Sequence
  Synthetic construct
RSRTPSLPTVVRTPPKSPSSC

SEQ ID NO: 1062 - protein, 21 aa, Artificial Sequence
  Synthetic construct
RSRTPSLPTVRTPPKSPSSAC

SEQ ID NO: 1063 - protein, 22 aa, Artificial Sequence
  Synthetic construct
RSRTPSLPTAVVRTPPKSPSSC

SEQ ID NO: 1064 - protein, 22 aa, Artificial Sequence
  Synthetic construct
RSRTPSLPTVVRTPPKSPSSAC

SEQ ID NO: 1065 - protein, 23 aa, Artificial Sequence
  Synthetic construct
RSRTPSLPTAVVRTPPKSPSSAC

SEQ ID NO: 1066 - protein, 19 aa, Artificial Sequence
  Synthetic construct
SRTPSLPTPVRTPPKSPSC

SEQ ID NO: 1067 - protein, 20 aa, Artificial Sequence
  Synthetic construct
SRTPSLPTPVVRTPPKSPSC

SEQ ID NO: 1068 - protein, 20 aa, Artificial Sequence
  Synthetic construct
SRTPSLPTPVRTPPKSPSSC

SEQ ID NO: 1069 - protein, 21 aa, Artificial Sequence
  Synthetic construct
SRTPSLPTPAVVRTPPKSPSC

SEQ ID NO: 1070 - protein, 21 aa, Artificial Sequence
  Synthetic construct
SRTPSLPTPVVRTPPKSPSSC

SEQ ID NO: 1071 - protein, 21 aa, Artificial Sequence
  Synthetic construct
SRTPSLPTPVRTPPKSPSSAC

SEQ ID NO: 1072 - protein, 22 aa, Artificial Sequence
  Synthetic construct
SRTPSLPTPAVVRTPPKSPSSC

SEQ ID NO: 1073 - protein, 22 aa, Artificial Sequence
  Synthetic construct
SRTPSLPTPVVRTPPKSPSSAC

SEQ ID NO: 1074 - protein, 23 aa, Artificial Sequence
  Synthetic construct
SRTPSLPTPAVVRTPPKSPSSAC

SEQ ID NO: 1075 - protein, 20 aa, Artificial Sequence
  Synthetic construct
SRSRTPSLPTVRTPPKSPSC

SEQ ID NO: 1076 - protein, 21 aa, Artificial Sequence
  Synthetic construct
SRSRTPSLPTVVRTPPKSPSC

SEQ ID NO: 1077 - protein, 21 aa, Artificial Sequence
  Synthetic construct
SRSRTPSLPTVRTPPKSPSSC

SEQ ID NO: 1078 - protein, 22 aa, Artificial Sequence
  Synthetic construct
SRSRTPSLPTAVVRTPPKSPSC

SEQ ID NO: 1079 - protein, 22 aa, Artificial Sequence
  Synthetic construct
SRSRTPSLPTVVRTPPKSPSSC

SEQ ID NO: 1080 - protein, 22 aa, Artificial Sequence
  Synthetic construct
SRSRTPSLPTVRTPPKSPSSAC

SEQ ID NO: 1081 - protein, 23 aa, Artificial Sequence
  Synthetic construct
SRSRTPSLPTAVVRTPPKSPSSC

SEQ ID NO: 1082 - protein, 23 aa, Artificial Sequence
  Synthetic construct
SRSRTPSLPTVVRTPPKSPSSAC

SEQ ID NO: 1083 - protein, 24 aa, Artificial Sequence
  Synthetic construct
SRSRTPSLPTAVVRTPPKSPSSAC

SEQ ID NO: 1084 - protein, 20 aa, Artificial Sequence
  Synthetic construct
RSRTPSLPTPVRTPPKSPSC

SEQ ID NO: 1085 - protein, 21 aa, Artificial Sequence
  Synthetic construct
RSRTPSLPTPVVRTPPKSPSC

SEQ ID NO: 1086 - protein, 21 aa, Artificial Sequence
  Synthetic construct
RSRTPSLPTPVRTPPKSPSSC

SEQ ID NO: 1087 - protein, 22 aa, Artificial Sequence
  Synthetic construct
RSRTPSLPTPAVVRTPPKSPSC

SEQ ID NO: 1088 - protein, 22 aa, Artificial Sequence
  Synthetic construct
RSRTPSLPTPVVRTPPKSPSSC

SEQ ID NO: 1089 - protein, 22 aa, Artificial Sequence
  Synthetic construct
RSRTPSLPTPVRTPPKSPSSAC

SEQ ID NO: 1090 - protein, 23 aa, Artificial Sequence
  Synthetic construct
RSRTPSLPTPAVVRTPPKSPSSC

SEQ ID NO: 1091 - protein, 23 aa, Artificial Sequence
  Synthetic construct
RSRTPSLPTPVVRTPPKSPSSAC

SEQ ID NO: 1092 - protein, 24 aa, Artificial Sequence
  Synthetic construct
RSRTPSLPTPAVVRTPPKSPSSAC

SEQ ID NO: 1093 - protein, 21 aa, Artificial Sequence
  Synthetic construct
SRSRTPSLPTPVRTPPKSPSC

SEQ ID NO: 1094 - protein, 22 aa, Artificial Sequence
  Synthetic construct
SRSRTPSLPTPVVRTPPKSPSC

SEQ ID NO: 1095 - protein, 22 aa, Artificial Sequence
  Synthetic construct
SRSRTPSLPTPVRTPPKSPSSC

SEQ ID NO: 1096 - protein, 23 aa, Artificial Sequence
  Synthetic construct
SRSRTPSLPTPAVVRTPPKSPSC

SEQ ID NO: 1097 - protein, 23 aa, Artificial Sequence
  Synthetic construct
SRSRTPSLPTPVVRTPPKSPSSC

SEQ ID NO: 1098 - protein, 23 aa, Artificial Sequence
  Synthetic construct
SRSRTPSLPTPVRTPPKSPSSAC

SEQ ID NO: 1099 - protein, 24 aa, Artificial Sequence
  Synthetic construct
SRSRTPSLPTPAVVRTPPKSPSSC

SEQ ID NO: 1100 - protein, 24 aa, Artificial Sequence
  Synthetic construct
SRSRTPSLPTPVVRTPPKSPSSAC

SEQ ID NO: 1101 - protein, 25 aa, Artificial Sequence
  Synthetic construct
SRSRTPSLPTPAVVRTPPKSPSSAC

SEQ ID NO: 1102 - protein, 22 aa, Artificial Sequence
  Synthetic construct
SRTPSLPPSSAKSRSKIGSTEC

SEQ ID NO: 1103 - protein, 23 aa, Artificial Sequence
  Synthetic construct
SRTPSLPSPSSAKSRSKIGSTEC

SEQ ID NO: 1104 - protein, 23 aa, Artificial Sequence
  Synthetic construct
SRTPSLPPSSAKSRSKIGSTENC

SEQ ID NO: 1105 - protein, 24 aa, Artificial Sequence
  Synthetic construct
SRTPSLPKSPSSAKSRSKIGSTEC

SEQ ID NO: 1106 - protein, 24 aa, Artificial Sequence
  Synthetic construct
SRTPSLPSPSSAKSRSKIGSTENC

SEQ ID NO: 1107 - protein, 24 aa, Artificial Sequence
  Synthetic construct
SRTPSLPPSSAKSRSKIGSTENLC

SEQ ID NO: 1108 - protein, 25 aa, Artificial Sequence
  Synthetic construct
SRTPSLPKSPSSAKSRSKIGSTENC

SEQ ID NO: 1109 - protein, 25 aa, Artificial Sequence
  Synthetic construct
SRTPSLPSPSSAKSRSKIGSTENLC

SEQ ID NO: 1110 - protein, 26 aa, Artificial Sequence
  Synthetic construct
SRTPSLPKSPSSAKSRSKIGSTENLC

SEQ ID NO: 1111 - protein, 23 aa, Artificial Sequence
  Synthetic construct
RSRTPSLPPSSAKSRSKIGSTEC

SEQ ID NO: 1112 - protein, 24 aa, Artificial Sequence
  Synthetic construct
RSRTPSLPSPSSAKSRSKIGSTEC

SEQ ID NO: 1113 - protein, 24 aa, Artificial Sequence
  Synthetic construct
RSRTPSLPPSSAKSRSKIGSTENC

SEQ ID NO: 1114 - protein, 25 aa, Artificial Sequence
  Synthetic construct
RSRTPSLPKSPSSAKSRSKIGSTEC

SEQ ID NO: 1115 - protein, 25 aa, Artificial Sequence
  Synthetic construct
RSRTPSLPSPSSAKSRSKIGSTENC

SEQ ID NO: 1116 - protein, 25 aa, Artificial Sequence
  Synthetic construct
RSRTPSLPPSSAKSRSKIGSTENLC

SEQ ID NO: 1117 - protein, 26 aa, Artificial Sequence
  Synthetic construct
RSRTPSLPKSPSSAKSRSKIGSTENC

SEQ ID NO: 1118 - protein, 26 aa, Artificial Sequence
  Synthetic construct
RSRTPSLPSPSSAKSRSKIGSTENLC

SEQ ID NO: 1119 - protein, 27 aa, Artificial Sequence
  Synthetic construct
RSRTPSLPKSPSSAKSRSKIGSTENLC

SEQ ID NO: 1120 - protein, 23 aa, Artificial Sequence
  Synthetic construct
SRTPSLPTPSSAKSRSKIGSTEC

SEQ ID NO: 1121 - protein, 24 aa, Artificial Sequence
  Synthetic construct
SRTPSLPTSPSSAKSRSKIGSTEC

SEQ ID NO: 1122 - protein, 24 aa, Artificial Sequence
  Synthetic construct
SRTPSLPTPSSAKSRSKIGSTENC

SEQ ID NO: 1123 - protein, 25 aa, Artificial Sequence
  Synthetic construct
SRTPSLPTKSPSSAKSRSKIGSTEC

SEQ ID NO: 1124 - protein, 25 aa, Artificial Sequence
  Synthetic construct
SRTPSLPTSPSSAKSRSKIGSTENC

SEQ ID NO: 1125 - protein, 25 aa, Artificial Sequence
  Synthetic construct
SRTPSLPTPSSAKSRSKIGSTENLC

SEQ ID NO: 1126 - protein, 26 aa, Artificial Sequence
  Synthetic construct
SRTPSLPTKSPSSAKSRSKIGSTENC

SEQ ID NO: 1127 - protein, 26 aa, Artificial Sequence
  Synthetic construct
SRTPSLPTSPSSAKSRSKIGSTENLC

SEQ ID NO: 1128 - protein, 27 aa, Artificial Sequence
  Synthetic construct
SRTPSLPTKSPSSAKSRSKIGSTENLC

SEQ ID NO: 1129 - protein, 24 aa, Artificial Sequence
  Synthetic construct
SRSRTPSLPPSSAKSRSKIGSTEC

SEQ ID NO: 1130 - protein, 25 aa, Artificial Sequence
  Synthetic construct
SRSRTPSLPSPSSAKSRSKIGSTEC

SEQ ID NO: 1131 - protein, 25 aa, Artificial Sequence
  Synthetic construct
SRSRTPSLPPSSAKSRSKIGSTENC

SEQ ID NO: 1132 - protein, 26 aa, Artificial Sequence
  Synthetic construct
SRSRTPSLPKSPSSAKSRSKIGSTEC

SEQ ID NO: 1133 - protein, 26 aa, Artificial Sequence
  Synthetic construct
SRSRTPSLPSPSSAKSRSKIGSTENC

SEQ ID NO: 1134 - protein, 26 aa, Artificial Sequence
  Synthetic construct
SRSRTPSLPPSSAKSRSKIGSTENLC

SEQ ID NO: 1135 - protein, 27 aa, Artificial Sequence
  Synthetic construct
SRSRTPSLPKSPSSAKSRSKIGSTENC

SEQ ID NO: 1136 - protein, 27 aa, Artificial Sequence
  Synthetic construct
SRSRTPSLPSPSSAKSRSKIGSTENLC

SEQ ID NO: 1137 - protein, 28 aa, Artificial Sequence
  Synthetic construct
SRSRTPSLPKSPSSAKSRSKIGSTENLC

SEQ ID NO: 1138 - protein, 24 aa, Artificial Sequence
  Synthetic construct
RSRTPSLPTPSSAKSRSKIGSTEC

SEQ ID NO: 1139 - protein, 25 aa, Artificial Sequence
  Synthetic construct
RSRTPSLPTSPSSAKSRSKIGSTEC

SEQ ID NO: 1140 - protein, 25 aa, Artificial Sequence
  Synthetic construct
RSRTPSLPTPSSAKSRSKIGSTENC

SEQ ID NO: 1141 - protein, 26 aa, Artificial Sequence
  Synthetic construct
RSRTPSLPTKSPSSAKSRSKIGSTEC

SEQ ID NO: 1142 - protein, 26 aa, Artificial Sequence
  Synthetic construct
RSRTPSLPTSPSSAKSRSKIGSTENC

SEQ ID NO: 1143 - protein, 26 aa, Artificial Sequence
  Synthetic construct
RSRTPSLPTPSSAKSRSKIGSTENLC

SEQ ID NO: 1144 - protein, 27 aa, Artificial Sequence
  Synthetic construct
RSRTPSLPTKSPSSAKSRSKIGSTENC

SEQ ID NO: 1145 - protein, 27 aa, Artificial Sequence
  Synthetic construct
RSRTPSLPTSPSSAKSRSKIGSTENLC

SEQ ID NO: 1146 - protein, 28 aa, Artificial Sequence
  Synthetic construct
RSRTPSLPTKSPSSAKSRSKIGSTENLC

SEQ ID NO: 1147 - protein, 24 aa, Artificial Sequence
  Synthetic construct
SRTPSLPTPPSSAKSRSKIGSTEC

SEQ ID NO: 1148 - protein, 25 aa, Artificial Sequence
  Synthetic construct
SRTPSLPTPSPSSAKSRSKIGSTEC

SEQ ID NO: 1149 - protein, 25 aa, Artificial Sequence
  Synthetic construct
SRTPSLPTPPSSAKSRSKIGSTENC

SEQ ID NO: 1150 - protein, 26 aa, Artificial Sequence
  Synthetic construct
SRTPSLPTPKSPSSAKSRSKIGSTEC

SEQ ID NO: 1151 - protein, 26 aa, Artificial Sequence
  Synthetic construct
SRTPSLPTPSPSSAKSRSKIGSTENC

SEQ ID NO: 1152 - protein, 26 aa, Artificial Sequence
  Synthetic construct
SRTPSLPTPPSSAKSRSKIGSTENLC

SEQ ID NO: 1153 - protein, 27 aa, Artificial Sequence
  Synthetic construct
SRTPSLPTPKSPSSAKSRSKIGSTENC

SEQ ID NO: 1154 - protein, 27 aa, Artificial Sequence
  Synthetic construct
SRTPSLPTPSPSSAKSRSKIGSTENLC

SEQ ID NO: 1155 - protein, 28 aa, Artificial Sequence
  Synthetic construct
SRTPSLPTPKSPSSAKSRSKIGSTENLC

SEQ ID NO: 1156 - protein, 25 aa, Artificial Sequence
  Synthetic construct
SRSRTPSLPTPSSAKSRSKIGSTEC

SEQ ID NO: 1157 - protein, 26 aa, Artificial Sequence
  Synthetic construct
SRSRTPSLPTSPSSAKSRSKIGSTEC

SEQ ID NO: 1158 - protein, 26 aa, Artificial Sequence
  Synthetic construct
SRSRTPSLPTPSSAKSRSKIGSTENC

SEQ ID NO: 1159 - protein, 27 aa, Artificial Sequence
  Synthetic construct
SRSRTPSLPTKSPSSAKSRSKIGSTEC

SEQ ID NO: 1160 - protein, 27 aa, Artificial Sequence
  Synthetic construct
SRSRTPSLPTSPSSAKSRSKIGSTENC

SEQ ID NO: 1161 - protein, 27 aa, Artificial Sequence
  Synthetic construct
SRSRTPSLPTPSSAKSRSKIGSTENLC

SEQ ID NO: 1162 - protein, 28 aa, Artificial Sequence
  Synthetic construct
SRSRTPSLPTKSPSSAKSRSKIGSTENC

SEQ ID NO: 1163 - protein, 28 aa, Artificial Sequence
  Synthetic construct
SRSRTPSLPTSPSSAKSRSKIGSTENLC

SEQ ID NO: 1164 - protein, 29 aa, Artificial Sequence
  Synthetic construct
SRSRTPSLPTKSPSSAKSRSKIGSTENLC

SEQ ID NO: 1165 - protein, 25 aa, Artificial Sequence
  Synthetic construct
RSRTPSLPTPPSSAKSRSKIGSTEC

SEQ ID NO: 1166 - protein, 26 aa, Artificial Sequence
  Synthetic construct
RSRTPSLPTPSPSSAKSRSKIGSTEC

SEQ ID NO: 1167 - protein, 26 aa, Artificial Sequence
  Synthetic construct
RSRTPSLPTPPSSAKSRSKIGSTENC

SEQ ID NO: 1168 - protein, 27 aa, Artificial Sequence
  Synthetic construct
RSRTPSLPTPKSPSSAKSRSKIGSTEC

SEQ ID NO: 1169 - protein, 27 aa, Artificial Sequence
  Synthetic construct
RSRTPSLPTPSPSSAKSRSKIGSTENC

SEQ ID NO: 1170 - protein, 27 aa, Artificial Sequence
  Synthetic construct
RSRTPSLPTPPSSAKSRSKIGSTENLC

SEQ ID NO: 1171 - protein, 28 aa, Artificial Sequence
  Synthetic construct
RSRTPSLPTPKSPSSAKSRSKIGSTENC

SEQ ID NO: 1172 - protein, 28 aa, Artificial Sequence
  Synthetic construct
RSRTPSLPTPSPSSAKSRSKIGSTENLC

SEQ ID NO: 1173 - protein, 29 aa, Artificial Sequence
  Synthetic construct
RSRTPSLPTPKSPSSAKSRSKIGSTENLC

SEQ ID NO: 1174 - protein, 26 aa, Artificial Sequence
  Synthetic construct
SRSRTPSLPTPPSSAKSRSKIGSTEC

SEQ ID NO: 1175 - protein, 27 aa, Artificial Sequence
  Synthetic construct
SRSRTPSLPTPSPSSAKSRSKIGSTEC

SEQ ID NO: 1176 - protein, 27 aa, Artificial Sequence
  Synthetic construct
SRSRTPSLPTPPSSAKSRSKIGSTENC

SEQ ID NO: 1177 - protein, 28 aa, Artificial Sequence
  Synthetic construct
SRSRTPSLPTPKSPSSAKSRSKIGSTEC

SEQ ID NO: 1178 - protein, 28 aa, Artificial Sequence
  Synthetic construct
SRSRTPSLPTPSPSSAKSRSKIGSTENC

SEQ ID NO: 1179 - protein, 28 aa, Artificial Sequence
  Synthetic construct
SRSRTPSLPTPPSSAKSRSKIGSTENLC

SEQ ID NO: 1180 - protein, 29 aa, Artificial Sequence
  Synthetic construct
SRSRTPSLPTPKSPSSAKSRSKIGSTENC

SEQ ID NO: 1181 - protein, 29 aa, Artificial Sequence
  Synthetic construct
SRSRTPSLPTPSPSSAKSRSKIGSTENLC

SEQ ID NO: 1182 - protein, 30 aa, Artificial Sequence
  Synthetic construct
SRSRTPSLPTPKSPSSAKSRSKIGSTENLC

SEQ ID NO: 1183 - protein, 20 aa, Artificial Sequence
  Synthetic construct
SRTPSLPKSPVVSGDTSPRC

SEQ ID NO: 1184 - protein, 21 aa, Artificial Sequence
  Synthetic construct
SRTPSLPYKSPVVSGDTSPRC

SEQ ID NO: 1185 - protein, 22 aa, Artificial Sequence
  Synthetic construct
SRTPSLPVYKSPVVSGDTSPRC

SEQ ID NO: 1186 - protein, 22 aa, Artificial Sequence
  Synthetic construct
SRTPSLPYKSPVVSGDTSPRHC

SEQ ID NO: 1187 - protein, 23 aa, Artificial Sequence
  Synthetic construct
SRTPSLPIVYKSPVVSGDTSPRC

SEQ ID NO: 1188 - protein, 23 aa, Artificial Sequence
  Synthetic construct
SRTPSLPVYKSPVVSGDTSPRHC

SEQ ID NO: 1189 - protein, 23 aa, Artificial Sequence
  Synthetic construct
SRTPSLPYKSPVVSGDTSPRHLC

SEQ ID NO: 1190 - protein, 24 aa, Artificial Sequence
  Synthetic construct
SRTPSLPIVYKSPVVSGDTSPRHC

SEQ ID NO: 1191 - protein, 24 aa, Artificial Sequence
  Synthetic construct
SRTPSLPVYKSPVVSGDTSPRHLC

SEQ ID NO: 1192 - protein, 25 aa, Artificial Sequence
  Synthetic construct
SRTPSLPIVYKSPVVSGDTSPRHLC

SEQ ID NO: 1193 - protein, 21 aa, Artificial Sequence
  Synthetic construct
RSRTPSLPKSPVVSGDTSPRC

SEQ ID NO: 1194 - protein, 22 aa, Artificial Sequence
  Synthetic construct
RSRTPSLPYKSPVVSGDTSPRC

SEQ ID NO: 1195 - protein, 23 aa, Artificial Sequence
  Synthetic construct
RSRTPSLPVYKSPVVSGDTSPRC

SEQ ID NO: 1196 - protein, 23 aa, Artificial Sequence
  Synthetic construct
RSRTPSLPYKSPVVSGDTSPRHC

SEQ ID NO: 1197 - protein, 24 aa, Artificial Sequence
  Synthetic construct
RSRTPSLPIVYKSPVVSGDTSPRC

SEQ ID NO: 1198 - protein, 24 aa, Artificial Sequence
  Synthetic construct
RSRTPSLPVYKSPVVSGDTSPRHC

SEQ ID NO: 1199 - protein, 24 aa, Artificial Sequence
  Synthetic construct
RSRTPSLPYKSPVVSGDTSPRHLC

SEQ ID NO: 1200 - protein, 25 aa, Artificial Sequence
  Synthetic construct
RSRTPSLPIVYKSPVVSGDTSPRHC

SEQ ID NO: 1201 - protein, 25 aa, Artificial Sequence
  Synthetic construct
RSRTPSLPVYKSPVVSGDTSPRHLC

SEQ ID NO: 1202 - protein, 26 aa, Artificial Sequence
  Synthetic construct
RSRTPSLPIVYKSPVVSGDTSPRHLC

SEQ ID NO: 1203 - protein, 21 aa, Artificial Sequence
  Synthetic construct
SRTPSLPTKSPVVSGDTSPRC

SEQ ID NO: 1204 - protein, 22 aa, Artificial Sequence
  Synthetic construct
SRTPSLPTYKSPVVSGDTSPRC

SEQ ID NO: 1205 - protein, 23 aa, Artificial Sequence
  Synthetic construct
SRTPSLPTVYKSPVVSGDTSPRC

SEQ ID NO: 1206 - protein, 23 aa, Artificial Sequence
  Synthetic construct
SRTPSLPTYKSPVVSGDTSPRHC

SEQ ID NO: 1207 - protein, 24 aa, Artificial Sequence
  Synthetic construct
SRTPSLPTIVYKSPVVSGDTSPRC

SEQ ID NO: 1208 - protein, 24 aa, Artificial Sequence
  Synthetic construct
SRTPSLPTVYKSPVVSGDTSPRHC

SEQ ID NO: 1209 - protein, 24 aa, Artificial Sequence
  Synthetic construct
SRTPSLPTYKSPVVSGDTSPRHLC

SEQ ID NO: 1210 - protein, 25 aa, Artificial Sequence
  Synthetic construct
SRTPSLPTIVYKSPVVSGDTSPRHC

SEQ ID NO: 1211 - protein, 25 aa, Artificial Sequence
  Synthetic construct
SRTPSLPTVYKSPVVSGDTSPRHLC

SEQ ID NO: 1212 - protein, 26 aa, Artificial Sequence
  Synthetic construct
SRTPSLPTIVYKSPVVSGDTSPRHLC

SEQ ID NO: 1213 - protein, 22 aa, Artificial Sequence
  Synthetic construct
SRSRTPSLPKSPVVSGDTSPRC

SEQ ID NO: 1214 - protein, 23 aa, Artificial Sequence
  Synthetic construct
SRSRTPSLPYKSPVVSGDTSPRC

SEQ ID NO: 1215 - protein, 24 aa, Artificial Sequence
  Synthetic construct
SRSRTPSLPVYKSPVVSGDTSPRC

SEQ ID NO: 1216 - protein, 24 aa, Artificial Sequence
  Synthetic construct
SRSRTPSLPYKSPVVSGDTSPRHC

SEQ ID NO: 1217 - protein, 25 aa, Artificial Sequence
  Synthetic construct
SRSRTPSLPIVYKSPVVSGDTSPRC

SEQ ID NO: 1218 - protein, 25 aa, Artificial Sequence
  Synthetic construct
SRSRTPSLPVYKSPVVSGDTSPRHC

SEQ ID NO: 1219 - protein, 25 aa, Artificial Sequence
  Synthetic construct
SRSRTPSLPYKSPVVSGDTSPRHLC

SEQ ID NO: 1220 - protein, 26 aa, Artificial Sequence
  Synthetic construct
SRSRTPSLPIVYKSPVVSGDTSPRHC

SEQ ID NO: 1221 - protein, 26 aa, Artificial Sequence
  Synthetic construct
SRSRTPSLPVYKSPVVSGDTSPRHLC

SEQ ID NO: 1222 - protein, 27 aa, Artificial Sequence
  Synthetic construct
SRSRTPSLPIVYKSPVVSGDTSPRHLC

SEQ ID NO: 1223 - protein, 22 aa, Artificial Sequence
  Synthetic construct
RSRTPSLPTKSPVVSGDTSPRC

SEQ ID NO: 1224 - protein, 23 aa, Artificial Sequence
  Synthetic construct
RSRTPSLPTYKSPVVSGDTSPRC

SEQ ID NO: 1225 - protein, 24 aa, Artificial Sequence
  Synthetic construct
RSRTPSLPTVYKSPVVSGDTSPRC

SEQ ID NO: 1226 - protein, 24 aa, Artificial Sequence
  Synthetic construct
RSRTPSLPTYKSPVVSGDTSPRHC

SEQ ID NO: 1227 - protein, 25 aa, Artificial Sequence
  Synthetic construct
RSRTPSLPTIVYKSPVVSGDTSPRC

SEQ ID NO: 1228 - protein, 25 aa, Artificial Sequence
  Synthetic construct
RSRTPSLPTVYKSPVVSGDTSPRHC

SEQ ID NO: 1229 - protein, 25 aa, Artificial Sequence
  Synthetic construct
RSRTPSLPTYKSPVVSGDTSPRHLC

SEQ ID NO: 1230 - protein, 26 aa, Artificial Sequence
  Synthetic construct
RSRTPSLPTIVYKSPVVSGDTSPRHC

SEQ ID NO: 1231 - protein, 26 aa, Artificial Sequence
  Synthetic construct
RSRTPSLPTVYKSPVVSGDTSPRHLC

SEQ ID NO: 1232 - protein, 27 aa, Artificial Sequence
  Synthetic construct
RSRTPSLPTIVYKSPVVSGDTSPRHLC

SEQ ID NO: 1233 - protein, 22 aa, Artificial Sequence
  Synthetic construct
SRTPSLPTPKSPVVSGDTSPRC

SEQ ID NO: 1234 - protein, 23 aa, Artificial Sequence
  Synthetic construct
SRTPSLPTPYKSPVVSGDTSPRC

SEQ ID NO: 1235 - protein, 24 aa, Artificial Sequence
  Synthetic construct
SRTPSLPTPVYKSPVVSGDTSPRC

SEQ ID NO: 1236 - protein, 24 aa, Artificial Sequence
  Synthetic construct
SRTPSLPTPYKSPVVSGDTSPRHC

SEQ ID NO: 1237 - protein, 25 aa, Artificial Sequence
  Synthetic construct
SRTPSLPTPIVYKSPVVSGDTSPRC

SEQ ID NO: 1238 - protein, 25 aa, Artificial Sequence
  Synthetic construct
SRTPSLPTPVYKSPVVSGDTSPRHC

SEQ ID NO: 1239 - protein, 25 aa, Artificial Sequence
  Synthetic construct
SRTPSLPTPYKSPVVSGDTSPRHLC

SEQ ID NO: 1240 - protein, 26 aa, Artificial Sequence
  Synthetic construct
SRTPSLPTPIVYKSPVVSGDTSPRHC

SEQ ID NO: 1241 - protein, 26 aa, Artificial Sequence
  Synthetic construct
SRTPSLPTPVYKSPVVSGDTSPRHLC

SEQ ID NO: 1242 - protein, 27 aa, Artificial Sequence
  Synthetic construct
SRTPSLPTPIVYKSPVVSGDTSPRHLC

SEQ ID NO: 1243 - protein, 23 aa, Artificial Sequence
  Synthetic construct
SRSRTPSLPTKSPVVSGDTSPRC

SEQ ID NO: 1244 - protein, 24 aa, Artificial Sequence
  Synthetic construct
SRSRTPSLPTYKSPVVSGDTSPRC

SEQ ID NO: 1245 - protein, 25 aa, Artificial Sequence
  Synthetic construct
SRSRTPSLPTVYKSPVVSGDTSPRC

SEQ ID NO: 1246 - protein, 25 aa, Artificial Sequence
  Synthetic construct
SRSRTPSLPTYKSPVVSGDTSPRHC

SEQ ID NO: 1247 - protein, 26 aa, Artificial Sequence
  Synthetic construct
SRSRTPSLPTIVYKSPVVSGDTSPRC

SEQ ID NO: 1248 - protein, 26 aa, Artificial Sequence
  Synthetic construct
SRSRTPSLPTVYKSPVVSGDTSPRHC

SEQ ID NO: 1249 - protein, 26 aa, Artificial Sequence
  Synthetic construct
SRSRTPSLPTYKSPVVSGDTSPRHLC

SEQ ID NO: 1250 - protein, 27 aa, Artificial Sequence
  Synthetic construct
SRSRTPSLPTIVYKSPVVSGDTSPRHC

SEQ ID NO: 1251 - protein, 27 aa, Artificial Sequence
  Synthetic construct
SRSRTPSLPTVYKSPVVSGDTSPRHLC

SEQ ID NO: 1252 - protein, 28 aa, Artificial Sequence
  Synthetic construct
SRSRTPSLPTIVYKSPVVSGDTSPRHLC

SEQ ID NO: 1253 - protein, 23 aa, Artificial Sequence
  Synthetic construct
RSRTPSLPTPKSPVVSGDTSPRC

SEQ ID NO: 1254 - protein, 24 aa, Artificial Sequence
  Synthetic construct
RSRTPSLPTPYKSPVVSGDTSPRC

SEQ ID NO: 1255 - protein, 25 aa, Artificial Sequence
  Synthetic construct
RSRTPSLPTPVYKSPVVSGDTSPRC

SEQ ID NO: 1256 - protein, 25 aa, Artificial Sequence
  Synthetic construct
RSRTPSLPTPYKSPVVSGDTSPRHC

SEQ ID NO: 1257 - protein, 26 aa, Artificial Sequence
  Synthetic construct
RSRTPSLPTPIVYKSPVVSGDTSPRC

SEQ ID NO: 1258 - protein, 26 aa, Artificial Sequence
  Synthetic construct
RSRTPSLPTPVYKSPVVSGDTSPRHC

SEQ ID NO: 1259 - protein, 26 aa, Artificial Sequence
  Synthetic construct
RSRTPSLPTPYKSPVVSGDTSPRHLC

SEQ ID NO: 1260 - protein, 27 aa, Artificial Sequence
  Synthetic construct
RSRTPSLPTPIVYKSPVVSGDTSPRHC

SEQ ID NO: 1261 - protein, 27 aa, Artificial Sequence
  Synthetic construct
RSRTPSLPTPVYKSPVVSGDTSPRHLC

SEQ ID NO: 1262 - protein, 28 aa, Artificial Sequence
  Synthetic construct
RSRTPSLPTPIVYKSPVVSGDTSPRHLC

SEQ ID NO: 1263 - protein, 24 aa, Artificial Sequence
  Synthetic construct
SRSRTPSLPTPKSPVVSGDTSPRC

SEQ ID NO: 1264 - protein, 25 aa, Artificial Sequence
  Synthetic construct
SRSRTPSLPTPYKSPVVSGDTSPRC

SEQ ID NO: 1265 - protein, 26 aa, Artificial Sequence
  Synthetic construct
SRSRTPSLPTPVYKSPVVSGDTSPRC

SEQ ID NO: 1266 - protein, 26 aa, Artificial Sequence
  Synthetic construct
SRSRTPSLPTPYKSPVVSGDTSPRHC

SEQ ID NO: 1267 - protein, 27 aa, Artificial Sequence
  Synthetic construct
SRSRTPSLPTPIVYKSPVVSGDTSPRC

SEQ ID NO: 1268 - protein, 27 aa, Artificial Sequence
  Synthetic construct
SRSRTPSLPTPVYKSPVVSGDTSPRHC

SEQ ID NO: 1269 - protein, 27 aa, Artificial Sequence
  Synthetic construct
SRSRTPSLPTPYKSPVVSGDTSPRHLC

SEQ ID NO: 1270 - protein, 28 aa, Artificial Sequence
  Synthetic construct
SRSRTPSLPTPIVYKSPVVSGDTSPRHC

SEQ ID NO: 1271 - protein, 28 aa, Artificial Sequence
  Synthetic construct
SRSRTPSLPTPVYKSPVVSGDTSPRHLC

SEQ ID NO: 1272 - protein, 29 aa, Artificial Sequence
  Synthetic construct
SRSRTPSLPTPIVYKSPVVSGDTSPRHLC

SEQ ID NO: 1273 - protein, 27 aa, Artificial Sequence
  Synthetic construct
PSSAKSRSKIGSTEKSPVVSGDTSPRC

SEQ ID NO: 1274 - protein, 28 aa, Artificial Sequence
  Synthetic construct
PSSAKSRSKIGSTEYKSPVVSGDTSPRC

SEQ ID NO: 1275 - protein, 29 aa, Artificial Sequence
  Synthetic construct
PSSAKSRSKIGSTEVYKSPVVSGDTSPRC

SEQ ID NO: 1276 - protein, 29 aa, Artificial Sequence
  Synthetic construct
PSSAKSRSKIGSTEYKSPVVSGDTSPRHC

SEQ ID NO: 1277 - protein, 30 aa, Artificial Sequence
  Synthetic construct
PSSAKSRSKIGSTEIVYKSPVVSGDTSPRC

SEQ ID NO: 1278 - protein, 30 aa, Artificial Sequence
  Synthetic construct
PSSAKSRSKIGSTEVYKSPVVSGDTSPRHC

SEQ ID NO: 1279 - protein, 30 aa, Artificial Sequence
  Synthetic construct
PSSAKSRSKIGSTEYKSPVVSGDTSPRHLC

SEQ ID NO: 1280 - protein, 31 aa, Artificial Sequence
  Synthetic construct
PSSAKSRSKIGSTEIVYKSPVVSGDTSPRHC

SEQ ID NO: 1281 - protein, 31 aa, Artificial Sequence
  Synthetic construct
PSSAKSRSKIGSTEVYKSPVVSGDTSPRHLC

SEQ ID NO: 1282 - protein, 32 aa, Artificial Sequence
  Synthetic construct
PSSAKSRSKIGSTEIVYKSPVVSGDTSPRHLC

SEQ ID NO: 1283 - protein, 28 aa, Artificial Sequence
  Synthetic construct
SPSSAKSRSKIGSTEKSPVVSGDTSPRC

SEQ ID NO: 1284 - protein, 29 aa, Artificial Sequence
  Synthetic construct
SPSSAKSRSKIGSTEYKSPVVSGDTSPRC

SEQ ID NO: 1285 - protein, 30 aa, Artificial Sequence
  Synthetic construct
SPSSAKSRSKIGSTEVYKSPVVSGDTSPRC

SEQ ID NO: 1286 - protein, 30 aa, Artificial Sequence
  Synthetic construct
SPSSAKSRSKIGSTEYKSPVVSGDTSPRHC

SEQ ID NO: 1287 - protein, 31 aa, Artificial Sequence
  Synthetic construct
SPSSAKSRSKIGSTEIVYKSPVVSGDTSPRC

SEQ ID NO: 1288 - protein, 31 aa, Artificial Sequence
  Synthetic construct
SPSSAKSRSKIGSTEVYKSPVVSGDTSPRHC

SEQ ID NO: 1289 - protein, 31 aa, Artificial Sequence
  Synthetic construct
SPSSAKSRSKIGSTEYKSPVVSGDTSPRHLC

SEQ ID NO: 1290 - protein, 32 aa, Artificial Sequence
  Synthetic construct
SPSSAKSRSKIGSTEIVYKSPVVSGDTSPRHC

SEQ ID NO: 1291 - protein, 32 aa, Artificial Sequence
  Synthetic construct
SPSSAKSRSKIGSTEVYKSPVVSGDTSPRHLC

SEQ ID NO: 1292 - protein, 28 aa, Artificial Sequence
  Synthetic construct
PSSAKSRSKIGSTENKSPVVSGDTSPRC

SEQ ID NO: 1293 - protein, 29 aa, Artificial Sequence
  Synthetic construct
PSSAKSRSKIGSTENYKSPVVSGDTSPRC

SEQ ID NO: 1294 - protein, 30 aa, Artificial Sequence
  Synthetic construct
PSSAKSRSKIGSTENVYKSPVVSGDTSPRC

SEQ ID NO: 1295 - protein, 30 aa, Artificial Sequence
  Synthetic construct
PSSAKSRSKIGSTENYKSPVVSGDTSPRHC

SEQ ID NO: 1296 - protein, 31 aa, Artificial Sequence
  Synthetic construct
PSSAKSRSKIGSTENIVYKSPVVSGDTSPRC

SEQ ID NO: 1297 - protein, 31 aa, Artificial Sequence
  Synthetic construct
PSSAKSRSKIGSTENVYKSPVVSGDTSPRHC

SEQ ID NO: 1298 - protein, 31 aa, Artificial Sequence
  Synthetic construct
PSSAKSRSKIGSTENYKSPVVSGDTSPRHLC

SEQ ID NO: 1299 - protein, 32 aa, Artificial Sequence
  Synthetic construct
PSSAKSRSKIGSTENIVYKSPVVSGDTSPRHC

SEQ ID NO: 1300 - protein, 32 aa, Artificial Sequence
  Synthetic construct
PSSAKSRSKIGSTENVYKSPVVSGDTSPRHLC

SEQ ID NO: 1301 - protein, 29 aa, Artificial Sequence
  Synthetic construct
KSPSSAKSRSKIGSTEKSPVVSGDTSPRC

SEQ ID NO: 1302 - protein, 30 aa, Artificial Sequence
  Synthetic construct
KSPSSAKSRSKIGSTEYKSPVVSGDTSPRC

SEQ ID NO: 1303 - protein, 31 aa, Artificial Sequence
  Synthetic construct
KSPSSAKSRSKIGSTEVYKSPVVSGDTSPRC

SEQ ID NO: 1304 - protein, 31 aa, Artificial Sequence
  Synthetic construct
KSPSSAKSRSKIGSTEYKSPVVSGDTSPRHC

SEQ ID NO: 1305 - protein, 32 aa, Artificial Sequence
  Synthetic construct
KSPSSAKSRSKIGSTEIVYKSPVVSGDTSPRC

SEQ ID NO: 1306 - protein, 32 aa, Artificial Sequence
  Synthetic construct
KSPSSAKSRSKIGSTEVYKSPVVSGDTSPRHC

SEQ ID NO: 1307 - protein, 32 aa, Artificial Sequence
  Synthetic construct
KSPSSAKSRSKIGSTEYKSPVVSGDTSPRHLC

SEQ ID NO: 1308 - protein, 29 aa, Artificial Sequence
  Synthetic construct
SPSSAKSRSKIGSTENKSPVVSGDTSPRC

SEQ ID NO: 1309 - protein, 30 aa, Artificial Sequence
  Synthetic construct
SPSSAKSRSKIGSTENYKSPVVSGDTSPRC

SEQ ID NO: 1310 - protein, 31 aa, Artificial Sequence
  Synthetic construct
SPSSAKSRSKIGSTENVYKSPVVSGDTSPRC

SEQ ID NO: 1311 - protein, 31 aa, Artificial Sequence
  Synthetic construct
SPSSAKSRSKIGSTENYKSPVVSGDTSPRHC

SEQ ID NO: 1312 - protein, 32 aa, Artificial Sequence
  Synthetic construct
SPSSAKSRSKIGSTENIVYKSPVVSGDTSPRC

SEQ ID NO: 1313 - protein, 32 aa, Artificial Sequence
  Synthetic construct
SPSSAKSRSKIGSTENVYKSPVVSGDTSPRHC

SEQ ID NO: 1314 - protein, 32 aa, Artificial Sequence
  Synthetic construct
SPSSAKSRSKIGSTENYKSPVVSGDTSPRHLC

SEQ ID NO: 1315 - protein, 29 aa, Artificial Sequence
  Synthetic construct
PSSAKSRSKIGSTENLKSPVVSGDTSPRC

SEQ ID NO: 1316 - protein, 30 aa, Artificial Sequence
  Synthetic construct
PSSAKSRSKIGSTENLYKSPVVSGDTSPRC

SEQ ID NO: 1317 - protein, 31 aa, Artificial Sequence
  Synthetic construct
PSSAKSRSKIGSTENLVYKSPVVSGDTSPRC

SEQ ID NO: 1318 - protein, 31 aa, Artificial Sequence
  Synthetic construct
PSSAKSRSKIGSTENLYKSPVVSGDTSPRHC

SEQ ID NO: 1319 - protein, 32 aa, Artificial Sequence
  Synthetic construct
PSSAKSRSKIGSTENLIVYKSPVVSGDTSPRC

SEQ ID NO: 1320 - protein, 32 aa, Artificial Sequence
  Synthetic construct
PSSAKSRSKIGSTENLVYKSPVVSGDTSPRHC

SEQ ID NO: 1321 - protein, 32 aa, Artificial Sequence
  Synthetic construct
PSSAKSRSKIGSTENLYKSPVVSGDTSPRHLC

SEQ ID NO: 1322 - protein, 30 aa, Artificial Sequence
  Synthetic construct
KSPSSAKSRSKIGSTENKSPVVSGDTSPRC

SEQ ID NO: 1323 - protein, 31 aa, Artificial Sequence
  Synthetic construct
KSPSSAKSRSKIGSTENYKSPVVSGDTSPRC

SEQ ID NO: 1324 - protein, 32 aa, Artificial Sequence
  Synthetic construct
KSPSSAKSRSKIGSTENVYKSPVVSGDTSPRC

SEQ ID NO: 1325 - protein, 32 aa, Artificial Sequence
  Synthetic construct
KSPSSAKSRSKIGSTENYKSPVVSGDTSPRHC

SEQ ID NO: 1326 - protein, 30 aa, Artificial Sequence
  Synthetic construct
SPSSAKSRSKIGSTENLKSPVVSGDTSPRC

SEQ ID NO: 1327 - protein, 31 aa, Artificial Sequence
  Synthetic construct
SPSSAKSRSKIGSTENLYKSPVVSGDTSPRC

SEQ ID NO: 1328 - protein, 32 aa, Artificial Sequence
  Synthetic construct
SPSSAKSRSKIGSTENLVYKSPVVSGDTSPRC

SEQ ID NO: 1329 - protein, 32 aa, Artificial Sequence
  Synthetic construct
SPSSAKSRSKIGSTENLYKSPVVSGDTSPRHC

SEQ ID NO: 1330 - protein, 31 aa, Artificial Sequence
  Synthetic construct
KSPSSAKSRSKIGSTENLKSPVVSGDTSPRC

SEQ ID NO: 1331 - protein, 32 aa, Artificial Sequence
  Synthetic construct
KSPSSAKSRSKIGSTENLYKSPVVSGDTSPRC

SEQ ID NO: 1332 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 1333 - protein, 317 aa, Artificial Sequence
  Synthetic construct
MKPFSVPILTVEEMTNSRFPIPLEKLFTGPSSAFVVQPQNGRCTTDGVLLGTTQLSPVNICTFRGDVTHIAGTQNYTMNLASQNWNNYDPTEEIPAPLGTPDFVGRIQGVLTQTTRRDGSTRGHKATVSTGSVHFTPKLGSVQFSTDTSNDFETGQNTRFTPVGVVQDGSTTHQNEPQQWVLPDYSGRDSHNVHLAPAVAPTFPGEQLLFFRSTMPGCSGYPNMNLDCLLPQEWVQHFYQESAPAQSDVALLRFVNPDTGRVLFECKLHKSGYVTVAHTGQHDLVIPPNGYFRFDSWVNQFYTLAPMGNGAGRRRAL

SEQ ID NO: 1334 - DNA, 59 bp, Artificial Sequence
  Synthetic construct
ctgtgaacatcgctactttccgcggcgacgtcacacacatcgcttgcacacaaaactac

SEQ ID NO: 1335 - DNA, 59 bp, Artificial Sequence
  Synthetic construct
gtagttttgtgtgcaagcgatgtgtgtgacgtcgccgcggaaagtagcgatgttcacag

SEQ ID NO: 1336 - DNA, 41 bp, Artificial Sequence
  Synthetic construct
gcaattcagcacagacacctgcaacgatttcgagactggcc

SEQ ID NO: 1337 - DNA, 41 bp, Artificial Sequence
  Synthetic construct
ggccagtctcgaaatcgttgcaggtgtctgtgctgaattgc

SEQ ID NO: 1338 - DNA, 42 bp, Artificial Sequence
  Synthetic construct
ccgtgggtgtcgttcaagactgcagcaccactcaccagaacg

SEQ ID NO: 1339 - DNA, 42 bp, Artificial Sequence
  Synthetic construct
cgttctggtgagtggtgctgcagtcttgaacgacacccacgg

SEQ ID NO: 1340 - DNA, 45 bp, Artificial Sequence
  Synthetic construct
gacgtcacacacatcgctggaaagacacaaaactacaccatgaac

SEQ ID NO: 1341 - DNA, 45 bp, Artificial Sequence
  Synthetic construct
gttcatggtgtagttttgtgtctttccagcgatgtgtgtgacgtc

SEQ ID NO: 1342 - DNA, 45 bp, Artificial Sequence
  Synthetic construct
caattcagcacagacacctcaaagaacgatttcgagactggccag

SEQ ID NO: 1343 - DNA, 45 bp, Artificial Sequence
  Synthetic construct
ctggccagtctcgaaatcgttctttgaggtgtctgtgctgaattg

SEQ ID NO: 1344 - DNA, 45 bp, Artificial Sequence
  Synthetic construct
gtgggtgtcgttcaagacggcaagagcaccactcaccagaacgaa

SEQ ID NO: 1345 - DNA, 45 bp, Artificial Sequence
  Synthetic construct
ttcgttctggtgagtggtgctcttgccgtcttgaacgacacccac

SEQ ID NO: 1346 - protein, 9 aa, Artificial Sequence
  Synthetic construct
HAGTYGLGD

SEQ ID NO: 1347 - protein, 12 aa, Artificial Sequence
  Synthetic construct
SSPGSPGTPGSR

SEQ ID NO: 1348 - protein, 11 aa, Artificial Sequence
  Synthetic construct
SRSRTPSLTTP

SEQ ID NO: 1349 - protein, 13 aa, Artificial Sequence
  Synthetic construct
AVVRTPPKSPSSA

SEQ ID NO: 1350 - protein, 9 aa, Artificial Sequence
  Synthetic construct
KSPSSAKSR

SEQ ID NO: 1351 - protein, 9 aa, Artificial Sequence
  Synthetic construct
SKIGSTENL

SEQ ID NO: 1352 - protein, 9 aa, Artificial Sequence
  Synthetic construct
IVYKSPVVS

SEQ ID NO: 1353 - protein, 9 aa, Artificial Sequence
  Synthetic construct
SGDTSPRHL

SEQ ID NO: 1354 - protein, 31 aa, Artificial Sequence
  Synthetic construct
SGYSSPGSPGTPGSRSRTPKSPVVSGDTSPR

SEQ ID NO: 1355 - protein, 27 aa, Artificial Sequence
  Synthetic construct
HAGTYGLGDKSPSSAKSRSKIGSTENL

SEQ ID NO: 1356 - protein, 24 aa, Artificial Sequence
  Synthetic construct
SRSRTPSLPTPAVVRTPPKSPSSA

SEQ ID NO: 1357 - protein, 318 aa, Artificial Sequence
  Synthetic construct
MGKPFSVPILTVEEMTNSRFPIPLEKLFTGPSSAFVVQPQNGRCTTDGVLLGTTQLSPVNICTFRGDVTHIAGCQNYTMNLASQNWNNYDPTEEIPAPLGTPDFVGRIQGVLTQTTRRDGSTRGHKATVSTGSVHFTPKLGSVQFSTDTCNDFETGQNTRFTPVGVVQDCSTTHQNEPQQWVLPDYSGRDSHNVHLAPAVAPTFPGEQLLFFRSTMPGCSGYPNMNLDCLLPQEWVQHFYQESAPAQSDVALLRFVNPDTGRVLFECKLHKSGYVTVAHTGQHDLVIPPNGYFRFDSWVNQFYTLAPMGNGAGRRRAL

SEQ ID NO: 1358 - DNA, 957 bp, Artificial Sequence
  Synthetic construct
atgggcaagcccttctcggtccctatcctgacagttgaggaaatgaccaactctagattcccaatcccgctggaaaagctcttcaccggaccctcctctgctttcgttgtgcagcctcaaaacggtcgttgcaccactgatggtgtcctgctcggcacaacccagctctcacctgtgaacatctgtactttccgcggcgacgtcacacacatcgctggatgccaaaactacaccatgaacttggcctcgcagaactggaacaactacgatccaaccgaggaaatccccgctcctttgggaactcccgacttcgtgggacgtatccaaggtgtcctgacacagactacacgtcgcgacggctctactcgcggacacaaggccactgtgtcgacaggttccgtccacttcacccctaagctgggctctgtgcaattcagcacagacacctgcaacgatttcgagactggccagaacaccaggttcactcccgtgggtgtcgttcaagactgcagcaccactcaccagaacgaaccccagcaatgggtcctccctgactactcgggcagggattcccacaacgttcacttggctcccgccgtggctccaaccttccctggagagcagttgctgttcttcagatccactatgccaggctgctctggatacccgaacatgaacctcgactgtctcttgcctcaggagtgggtgcaacacttctaccaggaatctgccccagctcaaagcgacgtcgctctgctccgtttcgttaaccccgataccggtcgcgtgctcttcgagtgtaagttgcacaagtctggttacgtcactgttgcccacacaggccagcacgacctggtcatccctcccaacggctacttccgcttcgatagctgggtcaaccagttctacacactcgccccgatgggaaacggagccggtcgtcgcagagccttgtaa

SEQ ID NO: 1359 - protein, 321 aa, Artificial Sequence
  Synthetic construct
MGKPFSVPILTVEEMTNSRFPIPLEKLFTGPSSAFVVQPQNGRCTTDGVLLGTTQLSPVNICTFRGDVTHIAGKTQNYTMNLASQNWNNYDPTEEIPAPLGTPDFVGRIQGVLTQTTRRDGSTRGHKATVSTGSVHFTPKLGSVQFSTDTSKNDFETGQNTRFTPVGVVQDGKSTTHQNEPQQWVLPDYSGRDSHNVHLAPAVAPTFPGEQLLFFRSTMPGCSGYPNMNLDCLLPQEWVQHFYQESAPAQSDVALLRFVNPDTGRVLFECKLHKSGYVTVAHTGQHDLVIPPNGYFRFDSWVNQFYTLAPMGNGAGRRRAL

SEQ ID NO: 1360 - DNA, 966 bp, Artificial Sequence
  Synthetic construct
atgggcaagcccttctcggtccctatcctgacagttgaggaaatgaccaactctagattcccaatcccgctggaaaagctcttcaccggaccctcctctgctttcgttgtgcagcctcaaaacggtcgttgcaccactgatggtgtcctgctcggcacaacccagctctcacctgtgaacatctgtactttccgcggcgacgtcacacacatcgctggaaagacacaaaactacaccatgaacttggcctcgcagaactggaacaactacgatccaaccgaggaaatccccgctcctttgggaactcccgacttcgtgggacgtatccaaggtgtcctgacacagactacacgtcgcgacggctctactcgcggacacaaggccactgtgtcgacaggttccgtccacttcacccctaagctgggctctgtgcaattcagcacagacacctcaaagaacgatttcgagactggccagaacaccaggttcactcccgtgggtgtcgttcaagacggcaagagcaccactcaccagaacgaaccccagcaatgggtcctccctgactactcgggcagggattcccacaacgttcacttggctcccgccgtggctccaaccttccctggagagcagttgctgttcttcagatccactatgccaggctgctctggatacccgaacatgaacctcgactgtctcttgcctcaggagtgggtgcaacacttctaccaggaatctgccccagctcaaagcgacgtcgctctgctccgtttcgttaaccccgataccggtcgcgtgctcttcgagtgtaagttgcacaagtctggttacgtcactgttgcccacacaggccagcacgacctggtcatccctcccaacggctacttccgcttcgatagctgggtcaaccagttctacacactcgccccgatgggaaacggagccggtcgtcgcagagccttgtaa

SEQ ID NO: 1361 - DNA, 47 bp, Artificial Sequence
  Synthetic construct
aactttaagaaggagatataccatgggcaagcccttctcggtcccta

SEQ ID NO: 1362 - DNA, 57 bp, Artificial Sequence
  Synthetic construct
cggatctcagtggtggtggtggtggtgctcgagttacaaggctctgcgacgaccggc

SEQ ID NO: 1363 - DNA, 696 bp, Artificial Sequence
  Synthetic construct
atgggtggtggtggttcttgcggcggcggcggctctgcgggcaatacgaactcgggcggctccacgacgaccatcaccaacaacaactcgggcaccaactcatccagcacgacctacacggttaaaagcggcgataccctgtggggtatttcacagcgttatggcatttcggttgctcagatccaaagcgcgaacaatctgaaatctaccattatctacatcggccaaaaactggtcctgaccggtagcgcgagctctacgaactcaggcggttcgaacaatagcgcttctaccacgccgaccacgtcggtgaccccggccaaaccgacgagccagaccaccgtcaaagtgaaatctggcgataccctgtgggccctgagtgttaaatataaaacgtccattgcacaactgaaatcatggaaccatctgagttccgacaccatttacatcggtcagaacctgatcgtcagtcaatccgcggccgcaagcaatccgtctaccggctcaggttcgaccgccacgaacaatagcaattctacgtcatcgaacagtaatgcatccattcacaaagtggttaaaggcgacaccctgtggggtctgagtcagaaaagtggttccccgattgcgtctatcaaagcatggaaccacctgagcagcgacacgattctgattggccagtatctgcgtattaaataa

SEQ ID NO: 1364 - protein, 230 aa, Artificial Sequence
  Synthetic construct
GGGGSCGGGGSAGNTNSGGSTTTITNNNSGTNSSSTTYTVKSGDTLWGISQRYGISVAQIQSANNLKSTIIYIGQKLVLTGSASSTNSGGSNNSASTTPTTSVTPAKPTSQTTVKVKSGDTLWALSVKYKTSIAQLKSWNHLSSDTIYIGQNLIVSQSAAASNPSTGSGSTATNNSNSTSSNSNASIHKVVKGDTLWGLSQKSGSPIASIKAWNHLSSDTILIGQYLRIK

SEQ ID NO: 1365 - DNA, 54 bp, Artificial Sequence
  Synthetic construct
tttaactttaagaaggagatatacatatgggtggtggtggttcttgcggcggcg

SEQ ID NO: 1366 - DNA, 53 bp, Artificial Sequence
  Synthetic construct
gtggtggtggtggtggtgctcgagttatttaatacgcagatactggccaatca

(19) United States
(12) Patent Application Publication
(10) Pub. No.: US 2024/0252606 A1
(43) Pub. Date: Aug. 1, 2024

(54) Phosphorylated Polypeptide Antigen Vaccine, Preparation Method Therefor and Application Thereof

(71) Applicants: CHANGCHUN BCHT BIOTECHNOLOGY CO., Changchun, Jilin Province (CN); JILIN UNIVERSITY, Changchun, Jilin Province (CN)

(72) Inventors: Wei KONG, Changchun (CN); Hui WU, Changchun (CN); Yao SUN, Changchun (CN); Yongqing GUO, Changchun (CN)

(73) Assignees: CHANGCHUN BCHT BIOTECHNOLOGY CO., Changchun, Jilin Province (CN); JILIN UNIVERSITY, Changchun, Jilin Province (CN)

(21) Appl. No.: 17/059,665

(22) PCT Filed: Mar. 26, 2019
(86) PCT No.: PCT/CN2019/079708

§ 371 Date: Nov. 30, 2020

(30) Foreign Application Priority Data
May 31, 2018 (CN) 201810551372.1

(51) Int. Cl. A61K 39/00; A61P 25/28; A61P 37/04

(52) U.S. Cl. CPC A61K 39/0007; A61P 25/28; A61P 37/04; A61K 2039/55561; A61K 2039/55566; A61K 2039/6031; A61K 2039/6037; A61K 2039/6068; A61K 2039/6075; A61K 2039/6081; A61K 2039/627

(57) ABSTRACT

The present invention discloses a phosphorylated polypeptide antigen vaccine, comprising at least two polypeptide fragments or conservatively modified variants thereof from human full-length Tau protein, wherein the polypeptide fragments or conservatively modified variants thereof contain phosphorylation sites. The present invention also discloses a complex vaccine formed by coupling a phosphorylated polypeptide antigen vaccine with a carrier. The polypeptide antigen vaccine and the complex vaccine can be used for preventing and/or treating tauopathy comprising Alzheimer's disease (AD).

### TECHNICAL FIELD

The present invention relates to the field of molecular biology. In particular, the present invention relates to splicing of truncated Tau proteins to form a phosphorylated polypeptide antigen vaccine. Preferably, this polypeptide antigen vaccine is connected with a suitable carrier to prepare a complex vaccine. The polypeptide antigen vaccine and the complex vaccine can be used for preventing and/or treating tauopathy comprising Alzheimer's disease (AD).

### BACKGROUND ART

Alzheimer's disease (AD) is a progressive neurodegenerative disease which causes deficits in cognitive function and declines in learning, memory, language, and motor functions in AD patients. With the progression of the disease, AD patients show concomitant behavioral, emotional, interpersonal, and social deterioration. Late-stage AD patients are unable to speak, understand language, and take care of themselves. Several drugs currently approved can control or relieve complications and side effects from AD and improve AD patients' life quality. Nevertheless, there is still an unmet need for treatments that directly target the disease process and have improved therapeutic effects.

AD is histologically characterized by the deposition of extraneuronal plaques, intracellular and extracellular neurofibrillary tangles and loss of neurons in the brain. The results of numerous studies demonstrate that Tau proteins play an important role in AD pathology and are also the most downstream change in the pathological changes of AD. Studies show that Aβ neurotoxicity in cultured neurons appears to depend on Tau proteins; and a reduction in the amount of Tau proteins in a model of tauopathy can restore memory function thereof. In addition, a reduction in endogenous Tau proteins inhibits behavioral deficits in transgenic mice that express the human amyloid precursor, without altering their Aβ levels. Thus, therapies targeting Tau proteins can become an effective strategy for treating tauopathy comprising AD.

Tau proteins are microtubule-associated proteins. Tau proteins mediate the assembly of tubulin monomers into microtubules (MT) that constitute the neuronal microtubules network; Tau proteins carry nutrients and transmitters on microtubules through phosphorylation and dephosphorylation, which is significant for the proper formation and executive function of neuronal circuits. The binding of Tau to MT is controlled by dynamic phosphorylation and dephosphorylation, as demonstrated by experiments in vitro and in non-neuronal cells. When being hyperphosphorylated, Tau proteins will aggregate with each other and detach from the MT, and the MT will lose their stability so as to disintegrate themselves, thereby causing formation of neurofibrillary tangles (NFT) and neuronal loss.

Notwithstanding AD's prevalence in human beings, its pathogenesis has not been fully elucidated so far, and the current research and treatment methods have not yet met the needs. As demonstrated by experiments conducted in a tauopathy mouse model by Asuni et al., mice vaccinated with Tau protein derived phospho-peptide showed a reduction in neurofibrillary tangles and functional improvements. Although small molecules of short peptides can often interact with immune response products, they usually cannot elicit a response alone. These peptide immunogens are also called “haptens”, which cannot, on their own, produce immunogenicity or cause the production of antibodies in the body, and can only be prepared into an immunogenic composition by coupling them with a suitable carrier. On the other hand, full-length recombinant Tau proteins expressed in a prokaryotic system do not seem to be suitable as a vaccine.

In view of the foregoing, there is a need in the art for the development of a method of effectively preventing and/or treating tauopathy.

### SUMMARY OF THE INVENTION

With resepct to the aforementioned drawbacks, the present invention, based on human full-length Tau protein, designs phosphorylated polypeptide antigen vaccines, and complex vaccines which are formed by coupling the phosphorylated polypeptide antigen vaccines with suitable carriers for preventing and/or treating tauopathy comprising Alzheimer's disease (AD). The vaccine of the present invention has good safety and high immunogenicity, and can induce the production of high-titer antibodies.

In particular, the first aspect of the present invention provides a phosphorylated polypeptide antigen vaccine, which comprises at least two polypeptide fragments or conservative modified variants thereof from human full-length Tau protein, wherein the polypeptide fragments or conservative modified variants thereof contain phosphorylation sites. In some embodiments, the phosphorylated polypeptide antigen vaccine contains an additional cysteine residue at its C′-terminal.

In the context of the present invention, the term “phosphorylated polypeptide antigen vaccine” means a phosphorylated polypeptide, and can be used interchangeably with the term “phosphorylated polypeptide”.

In some embodiments, the polypeptide fragments are derived from a phosphorylation modification site-rich region of human full-length Tau protein. In some preferred embodiments, the polypeptide fragments are derived from the following regions of human full-length Tau protein: amino acids at positions 14 to 22 of human full-length Tau protein, amino acids at positions 194 to 266 of human full-length Tau protein, and/or amino acids at positions 392 to 408 of human full-length Tau protein.

In some embodiments, the polypeptide fragments are connected directly by peptide bonds or connected by amino acid linkers. In preferred embodiments, the polypeptide fragments are connected directly by peptide bonds.

In some specific embodiments, the phosphorylation sites include two or more, preferably all phosphorylated amino acid sites corresponding to positions 18, 202, 205, 212, 214, 231, 235, 238, 262, 396 and 404 of the amino acid sequence of human full-length Tau protein, namely, 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄). In some preferred embodiments, 1 to 4, preferably 1 to 3, more preferably 1 to 2, and most preferably 1 Ser site of the phosphorylation sites are substituted by an aspartic acid and/or 1 to 4, preferably 1 to 3, more preferably 1 to 2, and most preferably 1 Thr site of the phosphorylation sites are substituted by a glutamic acid; the overall net charge of the polypeptide fragments or conservatively modified variants thereof thus obtained and the charge distribution on the molecules thereof remain substantially the same as those before the substitution. The simulation of phosphorylation by replacing phosphorylation sites with aspartic acids and/or glutamic acids is well known in the art.

In some embodiments, the conservatively modified variant of the polypeptide fragment contained in the phosphorylated polypeptide antigen vaccine of the present invention is a variant obtained by conservatively substitution of one or more amino acids, preferably 1 to 10 amino acids, more preferably 1 to 6 amino acids, more preferably 1 to 4 amino acids, more preferably 1 to 3 amino acids and most preferably 1 amino acid of the polypeptide fragment with functionally similar amino acids. The conservatively substitution is well known in the art and includes the following 6 groups of amino acids:
1) alanine (A), serine (S) and threonine (T);
2) aspartic acid (D) and glutamic acid (E);
3) asparagine (N) and glutamine (Q);
4) arginine (R) and lysine (K);
5) isoleucine (I), leucine (L), methionine (M) and valine (V); and
6) phenylalanine (F), tyrosine (Y) and tryptophan (W).

The overall net charge of the variant of the polypeptide fragment thus obtained and the charge distribution on the molecules thereof remain substantially the same as those before the substitution.

In some preferred embodiments, the phosphorylated polypeptide antigen vaccine has an amino acid sequence as represented by any one of SEQ ID NOs: 1-1331, and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄). In some embodiments, the phosphorylated polypeptide antigen vaccine has an amino acid sequence which has at least 80%, at least 85%, at least 90%, at least 95%, preferably at least 98%, more preferably at least 99% sequence identity to any one of SEQ ID NOs: 1-1331, and the phosphorylated polypeptide antigen vaccine has basically the same immunogenic activity as the original phosphorylated polypeptide antigen vaccine, and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄).

In a more preferred embodiment, the phosphorylated polypeptide antigen vaccine has an amino acid sequence as represented by any one of SEQ ID NO: 201, SEQ ID NO: 225, SEQ ID NO: 306, SEQ ID NO: 387, SEQ ID NO: 468, SEQ ID NO: 558, SEQ ID NO: 567, SEQ ID NO: 769, SEQ ID NO: 784, SEQ ID NO: 875, SEQ ID NO: 1020, SEQ ID NO: 1101, SEQ ID NO: 1182, SEQ ID NO: 1272, SEQ ID NO: 1313 and SEQ ID NO: 1330, and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄). In some embodiments, the phosphorylated polypeptide antigen vaccine has an amino acid sequence which has at least 80%, at least 85%, at least 90%, at least 95%, preferably at least 98%, and more preferably at least 99% sequence identity to any one of the above sequences, and the phosphorylated polypeptide antigen vaccine has basically the same immunogenic activity as the original phosphorylated polypeptide antigen vaccine, and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄).

In a particularly specific embodiment, the phosphorylated polypeptide antigen vaccine has an amino acid sequence as represented by any one of SEQ ID NO: 201, SEQ ID NO: 225, SEQ ID NO: 306, SEQ ID NO: 387, SEQ ID NO: 468, SEQ ID NO: 558, SEQ ID NO: 567, SEQ ID NO: 769, SEQ ID NO: 784, SEQ ID NO: 875, SEQ ID NO: 1020, SEQ ID NO: 1101, SEQ ID NO: 1182, SEQ ID NO: 1272, SEQ ID NO: 1313 and SEQ ID NO: 1330, and the amino acid sequence respectively contains the following phosphorylation sites: 18(P-Tyr₁₈), 202(P-Ser₂₀₂), 205(P-Thr₂₀₅); 18(P-Tyr₁₈), 202(P-Ser₂₀₂), 205(P-Thr₂₀₅); 18(P-Tyr₁₈), 212(P-Thr₂₁₂), 214(P-Ser₂₁₄); 18(P-Tyr₁₈), 231(P-Thr₂₃₁), 235(P-Ser₂₃₅); 18(P-Tyr₁₈), 238(P-Ser₂₃₈), 262(P-Ser₂₆₂); 18(P-Tyr₁₈), 396(P-Ser₃₉₆), 404(P-Ser₄₀₄); 202(P-Ser₂₀₂), 205(P-Thr₂₀₅), 231(P-Thr₂₃₁), 235(P-Ser₂₃₅); 202(P-Ser₂₀₂), 205(P-Thr₂₀₅), 231(P-Thr₂₃₁), 235(P-Ser₂₃₅); 202(P-Ser₂₀₂), 205(P-Thr₂₀₅), 238(P-Ser₂₃₈), 262(P-Ser₂₆₂); 202(P-Ser₂₀₂), 205(P-Thr₂₀₅), 396(P-Ser₃₉₆), 404(P-Ser₄₀₄); 202(P-Ser₂₀₂), 205(P-Thr₂₀₅), 396(P-Ser₃₉₆), 404(P-Ser₄₀₄); 212(P-Thr₂₁₂), 214(P-Ser₂₁₄), 231(P-Thr₂₃₁), 235(P-Ser₂₃₅); 212(P-Thr₂₁₂), 214(P-Ser₂₁₄), 238(P-Ser₂₃₈), 262(P-Ser₂₆₂); 212(P-Thr₂₁₂), 214(P-Ser₂₁₄), 396(P-Ser₃₉₆), 404(P-Ser₄₀₄); 238(P-Ser₂₃₈), 262(P-Ser₂₆₂), 396(P-Ser₃₉₆), 404(P-Ser₄₀₄); 238(P-Ser₂₃₈), 262(P-Ser₂₆₂), 396(P-Ser₃₉₆) and 404(P-Ser₄₀₄).

The second aspect of the present invention provides a complex vaccine formed by coupling the phosphorylated polypeptide antigen vaccine of the first aspect of the present invention with a carrier.

In some embodiments, the carrier is selected from the group comprising human serum albumin, keyhole limpet hemocyanin, bacterium-like particles (BLP), immunoglobulin molecules, thyroglobulin, ovalbumin, bovine serum albumin component V, influenza hemagglutinin, PAN-DR binding peptide (PADRE polypeptide), malaria circumsporozoite (CS) protein, hepatitis B surface antigen (HBₛAg₁₉₋₂₈), Heat Shock Protein (HSP) 65, Bacille Calmette-Guérin (BCG), cholera toxin, attenuated cholera toxin variants, diphtheria toxin, norovirus capsid P protein, recombinant Streptococcus C5a peptidase, Streptococcus pyogenes ORF1224, Streptococcus pyogenes ORF1664, Streptococcus pyogenes ORF2452, pneumolysin, attenuated pneumolysin toxicity variants, Chlamydia pneumoniae ORFT367, Chlamydia pneumoniae ORFT858, Tetanus toxoid, HIV gp120T1, microbial surface components recognizing adhesive matrix molecules (MSCRAMMS), growth factor/hormone and chemokines, etc. Complex vaccines formed by coupling or mixing with carriers can, to the greatest extent, stimulate the body to produce a specific immune response against phosphorylated polypeptides.

In some preferred embodiments, the carrier is norovirus capsid P protein.

The term “norovirus capsid P protein”, herein also referred to as P protein, refers to the P protein in the norovirus capsid proteins, which can self-assemble in vitro into a P particle. When used herein, P protein used at the gene level means a gene fragment, nucleotide sequence, plasmid, etc. encoding a P protein; P protein used at the protein level means a P protein monomer or polymer.

The term P particle (abbreviated as PP) refers to a protein particle formed by the in vitro self-assembly of P protein in norovirus. The most common form of P particle is a tetracosamer. When used herein, P particle (PP) is only used at the protein level and means the form of polymer (such as a tetracosamer), including various proteins used for property detection and immunization.

In some specific embodiments, one amino acid in each of the three loops in loop domain of the norovirus capsid P protein is mutated to a cysteine to facilitate chemical connection with the phosphorylated polypeptide vaccine. The norovirus capsid P protein modified with this mutation was named PP-3C (whose sequence is represented by SEQ ID NO: 1357), wherein the mutation would not result in a frameshift mutation of the norovirus P protein.

In other specific embodiments, a lysine is inserted into each of the three loops in loop domain of the norovirus P protein to facilitate chemical coupling with spliced phosphorylated polypeptide. The norovirus capsid P protein modified with this mutation was named PP-3K (whose sequence is represented by SEQ ID NO: 1359), wherein the mutation would not result in a frameshift mutation of the norovirus P protein.

In other preferred embodiments, the carrier is bacterium-like particles (BLP).

In some specific embodiments, the BLP is coupled with a phosphorylated polypeptide antigen vaccine by means of a protein adaptor (PA); specifically, the BLP is connected with a PA (a GGGGSCGGGGS sequence is added to the N-terminal of the PA) (i.e., C-PA) through covalent binding to obtain C-PA-BLP, and then C-PA-BLP is coupled with a phosphorylated polypeptide antigen vaccine which contains a cysteine at the C-terminal.

The term “bacterium-like particles (BLP)” refers to a new mucosal adjuvant which is obtained by hot acid treatment of Lactococcus lactis, and is an inanimate spherical particle with a Lactococcus lactis peptidoglycan shell as its main component. BLP particles, as a carrier for vaccine antigen components, can be effectively bound to the antigen and display the antigen on its surface. The peptidoglycan shell of BLP itself activates the innate immune system by interacting with Toll-like receptors to function as an adjuvant. BLP can be obtained by methods known in the art.

The term “protein adaptor (PA)” refers to PA protein, which is a full-length or truncated sequence in the Lactococcus lactis cell wall hydrolase ACMA cell wall binding region. The PA protein used in the present invention is a product (i.e., a C-PA protein) obtained by adding a GGGGSCGGGGS sequence to the N-terminal of the amino acid sequence from positions 219 to 437 of the Lactococcus lactis cell wall hydrolase ACMA (GenBank: U17696.1), and has a sequence as represented by SEQ ID NO: 1364.

The third aspect of the present invention provides a method for preparing the complex vaccine of the second aspect of the present invention, the method comprising the following steps:
1) artificially synthesizing the phosphorylated polypeptide antigen vaccine of the first aspect of the present invention;
2) preparing a carrier to be coupled with the phosphorylated polypeptide antigen vaccine;
3) mixing the phosphorylated polypeptide antigen vaccine with the carrier to preform coupling reaction; and
4) separating and purifying the conjugate obtained in 3), thereby obtaining a complex vaccine.

In some embodiments, the carrier in step 2) of the method is a norovirus capsid P protein, preferably PP-3C or PP-3K, and step 2) specifically includes
i) obtaining an expression vector comprising a nucleic acid encoding a PP-3C or PP-3K protein;
ii) transferring the expression vector into a recipient cell;
iii) expressing the PP-3C or PP-3K protein, and allowing it to self-assemble into a recombinant P particle in the recipient cell;
preferably, step 2) also comprises isolation and purification steps. In some specific embodiments, ion exchange chromatography and/or hydrophobic chromatography can be used for purification.

In some embodiments, in step 3) of the method, PP-3C is used as a vaccine carrier for coupling, a preferred buffer system is an ammonium bicarbonate buffer system, and a preferred pH ranges from 7.5 to 8.8; preferably, the phosphorylated polypeptide antigen vaccine and the carrier are mixed in a ratio of 10:1 to 100:1, and a preferred reaction temperature ranges from 2° C. to 10° C.

In some embodiments, in step 3) of the method, PP-3K is used as a vaccine carrier for coupling, a preferred buffer system is a phosphate buffer system, and a preferred pH ranges from 7.0 to 8.5; preferably, the phosphorylated polypeptide antigen vaccine and the carrier are mixed in a ratio of 10:1 to 100:1, and a preferred reaction temperature ranges from 2° C. to 25° C.

In other embodiments, the carrier in step 2) of the method is bacterium-like particles (BLP). In some embodiments, step 3) of the method specifically comprises i) obtaining a purified protein adaptor—C-PA protein (whose sequence is represented by SEQ ID NO: 1364); ii) connecting the carrier—bacterium-like particles (BLP)—obtained in step 2) with the C-PA protein to obtain C-PA-BLP; and iii) preforming coupling reaction of C-PA-BLP with the phosphorylated polypeptide antigen vaccine; wherein a Tris buffer system is used as a buffer system, and a preferred pH ranges from 7.2 to 8.8; a preferred C-PA protein concentration is 0.1 mg/mL to 1.5 mg/mL, and a preferred reaction temperature ranges from 2° C. to 30° C. In some embodiments, the buffer system used in step 3) of the method is an ammonium bicarbonate buffer system, and a preferred pH ranges from 7.5 to 8.8; preferably, the phosphorylated polypeptide antigen vaccine and the C-PA-BLP are mixed in a ratio of 10:1 to 100:1, and a preferred reaction temperature ranges from 2° C. to 10° C.

In some embodiments, step 4) of the method comprises removing carriers and polypeptide antigens which are not successfully connected by methods including centrifugation, dialysis, and ultrafiltration.

The fourth aspect of the present invention provides a vaccine composition comprising the phosphorylated polypeptide antigen vaccine of the first aspect of the present invention or the complex vaccine of the second aspect of the present invention. Preferably, the vaccine composition further comprises pharmaceutically acceptable adjuvants.

In some embodiments, the pharmaceutically acceptable adjuvants are selected from one or more of CpG, MF59, AS02, AS03, Freund's complete adjuvant and Freund's incomplete adjuvant.

The fifth aspect of the present invention provides use of the phosphorylated polypeptide antigen vaccine of the first aspect of the present invention or the complex vaccine of the second aspect of the present invention or the vaccine composition of the fourth aspect of the present invention for preparing a medicament for prevention and/or treatment of neurodegenerative disorders.

In the present inventimon, the “neurodegenerative disorders” include, but not limited to, AD, Creutzfeldt-Jacob Syndrome, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism syndrome-dementia syndrome, argyrophilic grain dementia, corticobasal degeneration, diffuse neurofibrillary tangles with calcification, frontotemporal dementia with parkinsonism syndrome linked to chromosome 17, Hallevorden-Spatz disease, multiple system atrophy, Niemann-Pick disease type C, Pick's disease, progressive subcortical gliosis and progressive supranuclear panencephalitis; preferably the “neurodegenerative disorder” is AD.

In some embodiments, the vaccine or vaccine composition is preferably immunized subcutaneously, intraperitoneally or intramuscularly, and more preferably immunized intramuscularly.

The sixth aspect of the present invention provides use of the phosphorylated polypeptide antigen vaccine of the first aspect of the present invention or the complex vaccine of the second aspect of the present invention or the vaccine composition of the fourth aspect of the present invention for preparing a medicament for maintaining or improving, preferably recovering, and more preferably completely recovering the cognitive memory of mammals, especially human beings.

The seventh aspect of the present invention provides a method for treating or preventing neurodegenerative disorders, comprising administering to a subject the phosphorylated polypeptide antigen vaccine of the first aspect of the present invention or the complex vaccine of the second aspect of the present invention or the vaccine composition of the fourth aspect of the present invention.

In some embodiments, the subject is a mammal, preferably a human.

In some embodiments, the vaccine or vaccine composition is preferably administered subcutaneously, intraperitoneally or intramuscularly, and more preferably administered intramuscularly.

The eighth aspect of the present invention provides a method for maintaining or improving, preferably recovering, and more preferably completely recovering the cognitive memory of a subject, the method comprising administering to the subject the phosphorylated polypeptide antigen vaccine of the first aspect of the present invention or the complex vaccine of the second aspect of the present invention or the vaccine composition of the fourth aspect of the present invention.

The ninth aspect of the present invention provides a norovirus capsid P protein to be coupled with the phosphorylated polypeptide antigen vaccine of the first aspect of the present invention, wherein one amino acid in each of the three loops in loop domain of the norovirus capsid P protein is mutated to a cysteine—the P protein thus obtained is called PP-3C (with a sequence as represented by SEQ ID NO: 1357), or a lysine is inserted into each of the three loops in loop domain of the norovirus capsid P protein—the P protein thus obtained is called PP-3K (with a sequence as represented by SEQ ID NO: 1359).

The tenth aspect of the present invention provides a method for preparing the norovirus capsid P protein of the ninth aspect of the present invention, comprising the following steps:
i) obtaining an expression vector comprising a nucleic acid encoding a PP-3C or PP-3K protein;
ii) transferring the expression vector into a recipient cell;
iii) expressing the PP-3C or PP-3K protein, and allowing it to self-assemble into a recombinant P particle in the recipient cell;
preferably, the method also comprises isolation and purification steps. In some specific embodiments, ion exchange chromatography and/or hydrophobic chromatography can be used for purification.

The eleventh aspect of the present invention provides a protein adaptor with a GGGGSCGGGGS sequence inserted at its N-terminal—the protein adaptor thus obtained is C-PA (whose sequence is represented by SEQ ID NO: 1364).

The twelfth aspect of the present invention provides a method for preparing the protein adaptor of the eleventh aspect of the present invention, comprising the following steps:
i) obtaining an expression vector comprising a nucleic acid encoding a C-PA protein;
ii) transferring the expression vector into a host cell; and
iii) expressing the C-PA protein, and obtaining the C-PA protein by isolating and purifying.

### BRIEF DESCRIPTION OF DRAWINGS

FIGS. 1A to 1E show the maps of the various recombinant plasmid constructs. FIG. 1A is a schematic diagram of pET26b-PP plasmid that has been constructed; FIG. 1B is a schematic diagram of pET26b-PP-3C plasmid that has been constructed; FIG. 1C is a schematic diagram of pET28a-PP plasmid that has been constructed; FIG. 1D is a schematic diagram of pET28a-PP-3K plasmid that has been constructed; FIG. 1E is a schematic diagram of pColdIV-PP-3C plasmid that has been constructed.

FIGS. 2A to 2C show the recombinant pET26b plasmid construct and the results of purification and expression. FIG. 2A is a schematic diagram of a PP-3C particle. FIG. 2B is a schematic diagram of identification of pET26b—PP-3C plasmid that has been constructed by double-enzyme digestion; as shown in FIG. 2B, the target band is at 1,000 bp. FIG. 2C is a diagram of native gel electrophoresis of PP-3C and PP-3K protein samples; as shown in FIG. 2C, both of the protein bands appeared above 250 kDa, and it is determined from analysis that they are in the form of a dodecamer and a tetracosamer.

FIGS. 3A to 3C show the recombinant pET28a plasmid construct and the results of purification and expression. FIG. 3A is a schematic diagram of a PP-3K particle. FIG. 3B is a schematic diagram of identification of pET28a-PP-3K plasmid that has been constructed by double-enzyme digestion; as shown in FIG. 3B, the target band is at 1,000 bp. FIG. 3C is a SDS-PAGE graph of the target protein elution peak protein sample; as shown in FIG. 3C, the target proteins were enriched at 35 kDa.

FIG. 4 shows an electron microscope image of recombinant pET28a-PP-3K protein particles.

FIG. 5 shows an electron microscope image of recombinant pET26b-PP-3C protein particles.

FIGS. 6A to 6H show the levels of IgG antibodies against different antigens produced in mice immunized with phosphorylated polypeptide antigen vaccines in combination with Freund's adjuvant. FIG. 6A shows the levels of anti-Tau14-22 (pY18) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6B shows the levels of anti-Tau198-209 (pS202/pT205) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6C shows the levels of anti-Tau208-218 (pT212/pS214) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6D shows the levels of anti-Tau227-239 (pS231/pS235) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6E shows the levels of anti-Tau234-242 (pS238) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6F shows the levels of anti-Tau258-266 (pS262) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6G shows the levels of anti-Tau392-400 (pS396) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice. FIG. 6H shows the levels of anti-Tau401-408 (pS404) IgG antibodies produced in WT mice immunized with the vaccines A1 to A16; each group contained 6 mice; each group of mice was immunized four times at two-week intervals using complete Freund's adjuvant for the first immunization and incomplete Freund's adjuvant for the second, third and fourth immunization; two weeks after the fourth immunization, the mice were subjected to blood sampling to obtain mouse serum for ELISA experiment; the results were expressed as the mean O.D.+SD values obtained by each group of mice.

FIGS. 7A to 7S show the results of immunization of mice with phosphorylated polypeptide antigens coupled with a norovirus P protein through different immunization routes. Each group contained 6 mice. Each group of mice was immunized four times at two-week intervals. In the fourth week, sixth week and eighth week after the first immunization, the mice were subjected to blood sampling to obtain serum for ELISA experiment. FIG. 7A shows the results of intramuscular immunization of WT mice with A1 coupled with a norovirus P protein; the contents of anti-Tau14-22 (pY18) IgG antibodies and anti-Tau198-209 (pS202/pT205) IgG antibodies in the mouse serum were detected by ELISA method; the results were expressed as the mean O.D.+SD values obtained at a dilution of 1/200 of the mouse serum. FIG. 7B shows the results of intramuscular immunization of WT mice with A5 coupled with a norovirus P protein; the contents of anti-Tau14-22 (pY18) IgG antibodies, anti-Tau234-242 (pS238) IgG antibodies and anti-Tau258-266 (pS262) IgG antibodies in the mouse serum were detected by ELISA method; the results were expressed as the mean O.D.+SD values obtained at a dilution of 1/200 of the mouse serum. FIG. 7C shows the results of intramuscular immunization of WT mice with A9 coupled with a norovirus P protein; the contents of anti-Tau198-209 (pS202/pT205) IgG antibodies, anti-Tau234-242 (pS238) IgG antibodies and anti-Tau258-266 (pS262) IgG antibodies in the mouse serum were detected by ELISA method; the results were expressed as the mean O.D.+SD values obtained at a dilution of 1/200 of the mouse serum. FIG. 7D shows the results of intramuscular immunization of WT mice with A10 coupled with a norovirus P protein; the contents of anti-Tau198-209 (pS202/pT205) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7E shows the results of intramuscular immunization of WT mice with A11 coupled with a norovirus P protein; the contents of anti-Tau198-209 (pS202/pT205) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7F shows the results of intramuscular immunization of WT mice with A12 coupled with a norovirus P protein; the contents of anti-Tau208-218 (pT212/pS214) IgG antibodies and anti-Tau227-239 (pS231/pS235) IgG antibodies in the mouse serum were detected by ELISA method; the results were expressed as the mean O.D.+SD values obtained at a dilution of 1/200 of the mouse serum. FIG. 7G shows the results of intramuscular immunization of WT mice with A15 coupled with a norovirus P protein; the contents of anti-Tau234-242 (pS238) IgG antibodies, anti-Tau258-266 (pS262) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7H shows the results of intraperitoneal immunization of WT mice with A2 coupled with a norovirus P protein; the contents of anti-Tau14-22 (pY18) IgG antibodies and anti-Tau198-209 (pS202/pT205) IgG antibodies in the mouse serum were detected by ELISA method; the results were expressed as the mean O.D.+SD values obtained at a dilution of 1/200 of the mouse serum. FIG. 7J shows the results of intraperitoneal immunization of WT mice with A3 coupled with a norovirus P protein; the contents of anti-Tau14-22 (pY18) IgG antibodies and anti-Tau208-218 (pT212/pS214) IgG antibodies in the mouse serum were detected by ELISA method; the results were expressed as the mean O.D.+SD values obtained at a dilution of 1/200 of the mouse serum. FIG. 7K shows the results of intraperitoneal immunization of WT mice with A7 coupled with a norovirus P protein; the contents of anti-Tau198-209 (pS202/pT205) IgG antibodies and anti-Tau227-239 (pS231/pS235) IgG antibodies in the mouse serum were detected by ELISA method; the results were expressed as the mean O.D.+SD values obtained at a dilution of 1/200 of the mouse serum. FIG. 7L shows the results of intraperitoneal immunization of WT mice with A11 coupled with a norovirus P protein; the contents of anti-Tau198-209 (pS202/pT205) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7M shows the results of intraperitoneal immunization of WT mice with A14 coupled with a norovirus P protein; the contents of anti-Tau208-218 (pT212/pS214) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7N shows the results of subcutaneous immunization of WT mice with A4 coupled with a norovirus P protein; the contents of anti-Tau14-22 (pY18) IgG antibodies and anti-Tau227-239 (pS231/pS235) IgG antibodies in the mouse serum were detected by ELISA method; the results were expressed as the mean O.D.+SD values obtained at a dilution of 1/200 of the mouse serum. FIG. 7O shows the results of subcutaneous immunization of WT mice with A6 coupled with a norovirus P protein; the contents of anti-Tau14-22 (pY18) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7P shows the results of subcutaneous immunization of WT mice with A8 coupled with a norovirus P protein; the contents of anti-Tau198-209 (pS202/pT205) IgG antibodies and anti-Tau227-239 (pS231/pS235) IgG antibodies in the mouse serum were detected by ELISA method; the results were expressed as the mean O.D.+SD values obtained at a dilution of 1/200 of the mouse serum. FIG. 7Q shows the results of subcutaneous immunization of WT mice with A11 coupled with a norovirus P protein; the contents of anti-Tau198-209 (pS202/pT205) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7R shows the results of subcutaneous immunization of WT mice with A13 coupled with a norovirus P protein; the contents of anti-Tau208-218 (pT212/pS214) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum. FIG. 7S shows the results of subcutaneous immunization of WT mice with A16 coupled with a norovirus P protein; the contents of anti-Tau234-242 (pS238) IgG antibodies, anti-Tau258-266 (pS262) IgG antibodies, anti-Tau392-400 (pS396) IgG antibodies and anti-Tau401-408 (pS404) IgG antibodies in the mouse serum were detected by ELISA method; the results were expressed as the mean O.D.+SD values obtained at a dilution of 1/200 of the mouse serum.

FIG. 8 show that a phosphorylated polypeptide antigen coupled with PP-3C was combined respectively with CpG, AS02, AS03, MF59 and AS02+CpG adjuvant and a phosphorylated polypeptide antigen coupled with PP-3K was combined with AS02+CpG adjuvant to form different combinations; wild mice were intramuscularly immunized respectively with the above different combinations at week 0, week 2, week 4, week 6 and week 12; blood was taken two weeks after every immunization to obtain serum. The phosphorylated polypeptide of SEQ ID NO: 1354 corresponding to phosphorylated amino acids at sites 202, 205, 396, and 404 of full-length Tau protein, was used as the coating antigen for ELISA experiment. The results were expressed as the concentrations (standardized by AT8 antibodies) of the antibodies of anti-phosphorylated polypeptide obtained in each group of mice.

FIG. 9A to 9F show the analysis of immunogenicity of phosphorylated polypeptide antigen A5 coupled with PP-3K and combined with AS02+CpG adjuvant in mice. FIG. 9A shows the results of immunization of WT mice with phosphorylated polypeptide antigen A5 coupled with PP-3K and combined with AS02+CpG adjuvant; the content of antibodies in the mouse serum against the phosphorylated polypeptide with a sequence of SEQ ID NO: 1355 was detected. The results were expressed as the mean O.D.+SD value obtained at different dilutions of the mouse serum. FIG. 9B shows the results of immunization of P301S mice with phosphorylated polypeptide antigen A5 coupled with PP-3K and combined with AS02+CpG adjuvant; the content of antibodies in the mouse serum against the phosphorylated polypeptide with a sequence of SEQ ID NO: 1355 was detected. The results were expressed as the mean O.D.+SD value obtained at different dilutions of the mouse serum. FIG. 9C shows the results of immunization of P301S mice with phosphorylated polypeptide antigen A11 coupled with PP-3C and combined with AS02+CpG adjuvant; the content of antibodies in the mouse serum against the phosphorylated polypeptide with a sequence of SEQ ID NO: 1354 was detected. The results were expressed as the mean O.D.+SD value obtained at different dilutions of the mouse serum. FIG. 9D shows the results of immunization of P301S mice with phosphorylated polypeptide antigen A11 coupled with PP-3K and combined with AS02+CpG adjuvant; the content of antibodies in the mouse serum against the phosphorylated polypeptide with a sequence of SEQ ID NO: 1354 was detected. The results were expressed as the mean O.D.+SD value obtained at different dilutions of the mouse serum. FIG. 9E shows the results of immunization of WT mice with phosphorylated polypeptide antigen A12 coupled with PP-3K and combined with AS02+CpG adjuvant; the content of antibodies in the mouse serum against the phosphorylated polypeptide with a sequence of SEQ ID NO: 1356 was detected. The results were expressed as the mean O.D.+SD value obtained at different dilutions of the mouse serum. FIG. 9F shows the results of immunization of P301S mice with phosphorylated polypeptide antigen A12 coupled with PP-3K and combined with AS02+CpG adjuvant; the content of antibodies in the mouse serum against the phosphorylated polypeptide with a sequence of SEQ ID NO: 1356 was detected. The results were expressed as the mean O.D.+SD value obtained at different dilutions of the mouse serum.

FIGS. 10A to 10H show the cellular immunity of the vaccines in P301S transgenic mice. FIG. 10A shows the ELISPOT results for PP-3K-A5 complex vaccine in P301S transgenic mice; the results were expressed as the number of IFN-γ spots generated per 10⁶ cells when the animal spleen cells in different immune groups were stimulated by a prokaryotically expressed full-length Tau protein stimulus. FIG. 10B shows the ELISPOT results for PP-3K-A5 complex vaccine in P301S transgenic mice; the results were expressed as the number of IFN-γ spots generated per 10⁶ cells when the animal spleen cells in different immune groups were stimulated by a nonphosphorylated A5 polypeptide stimulus. FIG. 10C shows the ELISPOT results for PP-3K-A11 complex vaccine in P301S transgenic mice; the results were expressed as the number of IFN-γ spots generated per 10⁶ cells when the animal spleen cells in different immune groups were stimulated by a prokaryotically expressed full-length Tau protein stimulus. FIG. 10D shows the ELISPOT results for PP-3K-A11 complex vaccine in P301S transgenic mice; the results were expressed as the number of IFN-γ spots generated per 10⁶ cells when the animal spleen cells in different immune groups were stimulated by a nonphosphorylated A11 polypeptide stimulus. FIG. 10E shows the ELISPOT results for PP-3C-A11 complex vaccine in P301S transgenic mice; the results were expressed as the number of IFN-γ spots generated per 10⁶ cells when the animal spleen cells in different immune groups were stimulated by a prokaryotically expressed full-length Tau protein stimulus. FIG. 10F shows the ELISPOT results for PP-3C-A11 complex vaccine in P301S transgenic mice; the results were expressed as the number of IFN-γ spots generated per 10⁶ cells when the animal spleen cells in different immune groups were stimulated by a nonphosphorylated A11 polypeptide stimulus. FIG. 10G shows the ELISPOT results for PP-3K-A12 complex vaccine in P301S transgenic mice; the results were expressed as the number of IFN-γ spots generated per 10⁶ cells when the animal spleen cells in different immune groups were stimulated by a prokaryotically expressed full-length Tau protein stimulus. FIG. 10H shows the ELISPOT results for PP-3K-A12 complex vaccine in P301S transgenic mice; the results were expressed as the number of IFN-γ spots generated per 10⁶ cells when the animal spleen cells in different immune groups were stimulated by a nonphosphorylated A12 polypeptide stimulus.

FIGS. 11A to 11D show the rota-rod behavior of the vaccines in P301S transgenic mice. FIG. 11A shows the results of the rota-rod experiment for PP-3K-A5 complex vaccine in P301S transgenic mice; the results are expressed as curves of the average time until mice fell from the rota-rod within 300 s versus the detection time points for animals in different immune groups. FIG. 11B shows the results of the rota-rod experiment for PP-3C-A11 complex vaccine in P301S transgenic mice; the results are expressed as curves of the average time until mice fell from the rota-rod within 300 s versus the detection time points for animals in different immune groups. FIG. 11C shows the results of the rota-rod experiment for PP-3K-A11 complex vaccine in P301S transgenic mice; the results are expressed as curves of the average time until mice fell from the rota-rod within 300 s versus the detection time points for animals in different immune groups. FIG. 11D shows the results of the rota-rod experiment for PP-3K-A12 complex vaccine in P301S transgenic mice; the results are expressed as curves of the average time until mice fell from the rota-rod within 300 s versus the detection time points for animals in different immune groups.

FIGS. 12A to 12D show the nesting behavior of the vaccines in P301S transgenic mice. FIG. 12A shows the results of the nesting test for PP-3K-A5 complex vaccine in P301S transgenic mice; the results are expressed as the average of the scores given by three persons for each mouse in the nesting test. The results show that the mice in the immune group are statistically significantly different from the PBS group. FIG. 12B shows the results of the nesting test for PP-3C-A11 complex vaccine in P301S transgenic mice; the results are expressed as the average of the scores given by three persons for each mouse in the nesting test. The results show that the mice in the immune group are statistically significantly different from the PBS group. FIG. 12C shows the results of the nesting test for PP-3K-A11 complex vaccine in P301S transgenic mice; the results are expressed as the average of the scores given by three persons for each mouse in the nesting test. The results show that the mice in the immune group are statistically significantly different from the PBS group. FIG. 12D shows the results of the nesting test for PP-3K-A12 complex vaccine in P301S transgenic mice; the results are expressed as the average of the scores given by three persons for each mouse in the nesting test. The results show that the mice in the immune group are statistically significantly different from the PBS group.

FIGS. 13A to 13D show schematic diagrams of the results of HPLC quantification of the phosphorylated polypeptides coupled with PP-3C/C-PA-BLP. FIG. 13A shows the HPLC diagram when the concentration of the standard was 0.03125 mg/mL; the peak labelled in the figure is the peak position of the standard. FIG. 13B exemplarily shows the HPLC diagram of the phosphorylated polypeptide dissociated from the phosphorylated polypeptide sample coupled with PP-3C prepared in Example 4-2; the peak labelled in the figure is the peak position of the sample. FIG. 13C exemplarily shows the HPLC diagram of the phosphorylated polypeptide dissociated from the phosphorylated polypeptide sample coupled with C-PA-BLP prepared in Example 33; the peak labelled in the figure is the peak position of the sample. FIG. 13D shows a graph of a standard curve of the concentration of the standard versus the peak area.

FIG. 14 show is a schematic diagram of pET28a-C-PA plasmid that has been constructed.

FIGS. 15A to 15B show the recombinant pET28a-C-PA plasmid construct and the results of purification and expression. FIG. 15A is a schematic diagram of identification of pET28a-C-PA plasmid that has been constructed by double-enzyme digestion; as shown in FIG. 15A, the target band is at 700 bp. FIG. 15B is a SDS-PAGE graph of a C-PA protein sample; as shown in FIG. 15B, the protein band appeared around 24 kDa.

### DETAILED DESCRIPTION OF THE INVENTION

The content of the present invention is illustrated below through specific embodiments. It should be understood that the specific embodiments are for illustrative purposes only, and do not mean that the content of the present invention is limited to the specific embodiments.

### Example 1: Preparation of Phosphorylated Polypeptide Antigen Vaccines

In this example, based on human full-length Tau protein (SEQ ID NO: 1332), the inventor designed the following 15 phosphorylated polypeptide antigen vaccines comprising two polypeptide fragments from the human full-length Tau protein; the specific sequence information of the 15 phosphorylated polypeptide antigen vaccines is shown in Table 1 below:

TABLE 1
Sequence information of the 15 phosphorylated polypeptide antigen vaccines

| | | | The corresponding full-length |
|---|---|---|---|
| Sequence | Vaccine | | Tau protein fragmets and |
| No. | No. | Sequences | phosphorylation sites |
| SEQ ID | A1 | HAGTYGLGDSSPGSP | Tau14-22[pY18], Tau198- |
| NO: 201 | | GTPGSRC | 209[pS202, pT205] |
| SEQ ID | A2 | HAGTYGLGDRSGYSS | Tau14-22[pY18], Tau194- |
| NO: 225 | | PGSPGTPGSRSRTPC | 213[pS202, pT205] |
| SEQ ID | A3 | HAGTYGLGDSRSRTP | Tau14-22[pY18], Tau208- |
| NO: 306 | | SLPTPC | 218[pT212, pS214] |
| SEQ ID | A4 | HAGTYGLGDAVVRTPP | Tau14-22[pY18], Tau227- |
| NO: 387 | | KSPSSAC | 239[pT231, pS235] |
| SEQ ID | A5 | HAGTYGLGDKSPSSA | Tau14-22[pY18], Tau234- |
| NO: 468 | | KSRSKIGSTENLC | 242[pS238], Tau258- |
| | | | 266[pS262] |
| SEQ ID | A6 | HAGTYGLGDIVYKSPV | Tau14-22[pY18], Tau392- |
| NO: 558 | | VSGDTSPRHLC | 408[pS396, pS404] |
| SEQ ID | A7 | SSPGSPGTPGSRAVV | Tau198-209[pS202, pT205], |
| NO: 567 | | RTPPKSPSSAC | Tau227-239[pT231, pS235] |
| SEQ ID | A8 | SGYSSPGSPGTPGSR | Tau195-213[pS202, pT205], |
| NO: 769 | | SRTPVVRTPPKSPSSAC | Tau228-239[pT231, pS235] |
| SEQ ID | A9 | SSPGSPGTPGSRKSP | Tau198-209[pS202, pT205], |
| NO: 784 | | SSAKSRSKIGSTENLC | Tau234-242[pS238], Tau258- |
| | | | 266[pS262] |
| SEQ ID | A10 | SSPGSPGTPGSRIVYK | Tau198-209[pS202, pT205], |
| NO: 875 | | SPVVSGDTSPRHLC | Tau395-406[pS396, pS404] |
| SEQ ID | A11 | SGYSSPGSPGTPGSR | Tau195-213[pS202, pT205], |
| NO: 1020 | | SRTPKSPVVSGDTSPRC | Tau395-406[PS396, pS404] |
| SEQ ID | A12 | SRSRTPSLPTPAVVRT | Tau208-218[pT212, pS214], |
| NO: 1101 | | PPKSPSSAC | Tau227-239[pT231, pS235] |
| SEQ ID | A13 | SRSRTPSLPTPKSPSS | Tau208-218[pT212, pS214], |
| NO: 1182 | | AKSRSKIGSTENLC | Tau234-242[pS238], Tau258- |
| | | | 266[pS262] |
| SEQ ID | A14 | SRSRTPSLPTPIVYKSP | Tau208-218[pT212, pS214], |
| NO: 1272 | | VVSGDTSPRHLC | Tau392-408[pS396, pS404] |
| SEQ ID | A15 | SPSSAKSRSKIGSTEN | Tau235-242[pS238], Tau258- |
| NO: 1313 | | VYKSPVVSGDTSPRH | 265[pS262], Tau393- |
| | | C | 407[pS396, pS404] |
| SEQ ID | A16 | KSPSSAKSRSKIGSTE | Tau234-242[pS238], Tau258- |
| NO: 1330 | | NLKSPVVSGDTSPRC | 266[pS262], Tau395- |
| | | | 406[pS396, pS404] |

The aforementioned 15 phosphorylated polypeptide antigen vaccines were synthesized and prepared by GL biochem (Shanghai) Ltd., and are presented in lyophilized form.

### Example 2: Preparation of a Cysteine-Modified Norovirus P Protein (PP-3C)

The preparation of norovirus P protein plasmid using pET26b(+) as a vector has been described in Chinese invention patent application 2015104155561. In this example, three point mutations were performed using this plasmid as a template, one amino acid in each of the three loop structures of the norovirus P particle was mutated to a cysteine, and the protein was expressed and purified.

### 2.1 Construction of PP-3C Plasmid

Three rounds of site-directed mutagenesis were performed by site-directed mutation method using the pET26b-P protein plasmid which has already been constructed in Chinese invention patent application 2015104155561. Respectively, 5′ATCGCTGGAACACAA3″ in loop1 was mutated to 5′ATCGCTTGCACACAA3′. The specific method was as follows:

SEQ ID NO: 1334 (forward):
CTGTGAACATCGCTACTTTCCGCGGCGACGTCACACACATCGCTTGCAC
ACAAAACTAC,
and
SEQ ID NO: 1335 (reverse):
GTAGTTTTGTGTGCAAGCGATGTGTGTGACGTCGCCGCGGAAAGTAGCG
ATGTTCACAG;

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing.

The plasmid obtained by the first round of point mutation was subjected to a second round of point mutation. 5′ACCTCAAACGAT3′ in loop2 was mutated to 5′ACCTGCAACGA3′. The specific method was as follows:

SEQ ID NO: 1336 (forward):
GCAATTCAGCACAGACACCTGCAACGATTTCGAGACTGGCC,
and
SEQ ID NO: 1337 (reverse):
GGCCAGTCTCGAAATCGTTGCAGGTGTCTGTGCTGAATTGC;

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min. and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing.

The plasmid obtained by the second round of point mutation was subjected to a third round of point mutation. 5′GACGGCAGCACC3′ in loop3 was mutated to 5′GACTGCAGCACC3′. The specific method was as follows:

SEQ ID NO: 1338 (forward):
CCGTGGGTGTCGTTCAAGACTGCAGCACCACTCACCAGAACG,
and
SEQ ID NO: 1339 (reverse):
CGTTCTGGTGAGTGGTGCTGCAGTCTTGAACGACACCCACGG;

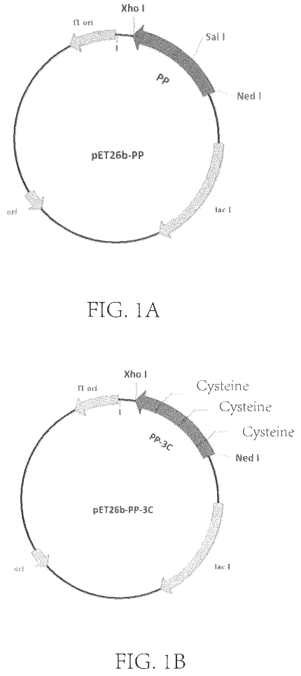

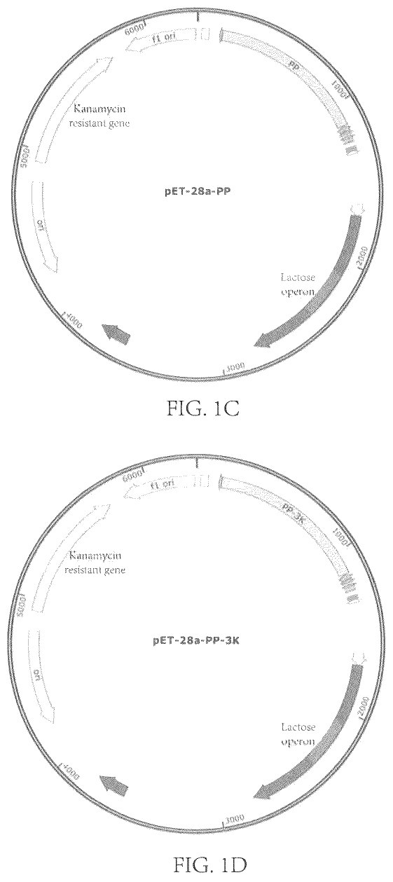

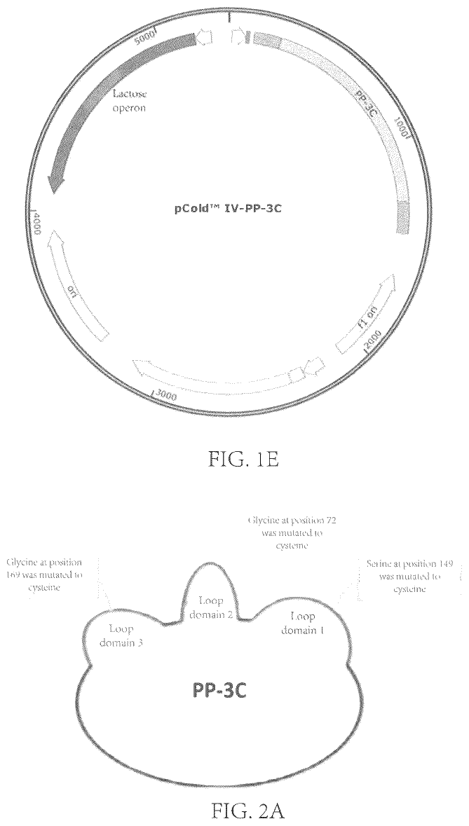

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 UM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min. and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing. The resulting plasmid of pET26b-PP-3C is shown in FIG. 1B.

2.2 Construction of the P Protein that has been Successfully Mutated, Namely the PP-3C Gene, on a pCold IV Carrier

An EcoR I restriction site was introduced at the 5′ end of the PP-3C gene (whose sequence is represented by SEQ ID NO: 1358) by PCR method, and a Hind Ill restriction site was introduced at the 3′ end. The specific method was as follows:

SEQ ID NO: 1340 (forward):
GGGAATTCCATATGAAGCCCTTCTCGGTCCCTATCCTGACAG,
and
SEQ ID NO: 1341 (reverse):
CCCAAGCTTTTACAAGGCTCTGCGACGACCGGCTC;

the pET26b-PP-3C plasmid obtained above was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 UM upstream primer and 0.3 μM downstream primer, 50 ng template DNA, 1 μL KOD enzyme, and water which was added to a final volume of 50 μL). PCR was carried out in accordance with the reaction system instructions to obtain 50 μL of PCR products. The products were subjected to agarose gel electrophoresis, and the fragments of interest were recovered using gel recovery kit (purchased from Tiangen Biotech Co., Ltd.) to obtain a PP-3C gene fragment with an EcoRI restriction site at the 5′ end and a Hind III restriction site at the 3′ end.

1 μL of Hind III enzyme and 1 μL of EcoR I enzyme (purchased from Takara Corporation) and 5 μL of enzyme digestion buffer (purchased from Takara Corporation) were added respectively to 2 μg of the aforementioned recovered fragment, and finally sterile water was added to the system to reach a final volume of 50 μL. Digestion was carried out at 37° C. for 2 hours. The products were purified using common DNA product purification kit (purchased from Tiangen Biotech Co., Ltd.) to obtain the fragment of interest having sticky ends.

1 μL of Hind III enzyme and 1 μL of EcoR I enzyme (purchased from Takara Corporation) and 5 μL of enzyme digestion buffer (purchased from Takara Corporation) were added respectively to 2 μg of pCold IV vector plasmid (purchased from Novagen Corporation), and finally sterile water was added to the system to reach a final volume of 50 μL. Digestion was carried out at 37° C. for 2 hours. The products were subjected to agarose gel electrophoresis, and the vector fragments were recovered using gel recovery kit (purchased from Tiangen Biotech Co., Ltd.) to obtain a plasmid vector having double enzyme-digested sticky ends

The above double enzyme-digested vector fragment and fragment of interest (with a molar ratio of 1:3, and a total volume of 15 μL) were mixed, and 0.75 μL of T4 ligase (purchased from Takara Corporation) and 1.5 μL of ligase buffer (purchased from Takara Corporation) were added. The ligation was carried out at 16° C. overnight. 10 μL of the ligated products were added to Tran1-Blue competent cells (purchased from Beijing TransGen Biotech, Ltd.), and placed on ice for 30 min. After that, the cells were subjected to heat shock at 42° C. for 45 s, placed on ice for 2 min, and then were added to 600 μL of fluid LB medium without resistance, and recovered at 200 rpm at 37° C. for 1 h. The culture broth was plated on a LB solid culture plate containing ampicillin (15 μg/mL), and were placed upside down at 37° C. overnight. The recombinant clones were obtained, and the sequence was verified by sequencing. A pColdIV-PP-3C plasmid that can express PP-3C protein was obtained, as shown in FIG. 1E.

### 2.3 Expression of the Cysteine-Modified Norovirus P Protein

1 μL of the recombinant plasmids prepared in the above examples were respectively added to 100 μL of E. coli BL21 competent cells (purchased from TransGen Corporation), ice-bathed for 30 min, heat shocked for 90 s in a water-bath at 42° C., and then ice-bathed for 2 min. 600 UL of LB medium was added to the mixture, and cultured at 180 rpm/min at 37° C. for 1 h. The mixture was coated evenly on a LB solid medium containing kanamycin (15 μg/mL) resistance and cultured at 37° C. for 24 h to obtain strains that can stably express recombinant proteins. A growing colony was picked and inoculated into 20 mL of LB medium. The mixture was cultured at 220 rpm at 37° C. When the OD value of the culture mixture reached 1.0, induction by Isopropyl β-D-Thiogalactoside (IPTG at a final concentration of 0.5 mmol/L) was carried out at 220 rpm at 16° C. overnight. After the induction, the culture broth was centrifuged at 4,000 rpm for 20 min. The supernatant was discarded, and the bacterial precipitates were resuspended with PBS. Centrifugation was conducted again at 4,000 rpm for 20 min and the supernatant was discarded to obtain the bacterial precipitates containing proteins of interest.

### 2.4 Purification of the Cysteine-Modified Norovirus P Protein

The bacterial precipitates obtained in 2.3 were resuspended by adding 20 mL of protein buffer (pH8.0, containing 50 mM Tris and 300 mM KCl). The bacteria were lysed by sonication on ice for 30 min. The mixture was centrifuged at 12,000 rpm at 4° C. for 30 min. Subsequently, the supernatant was taken and allowed to pass through 0.45 μm filter membrane to obtain crude extracts of proteins.

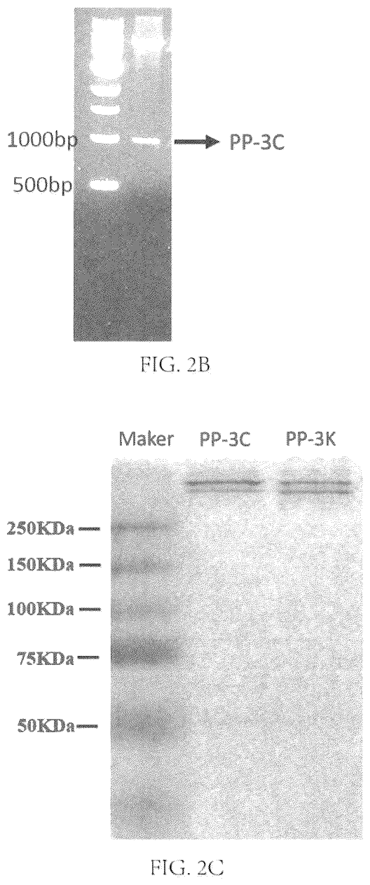

The structure of the recombinant PP-3C particle protein is shown in FIG. 2A.

The crude extracts of proteins were purified using an anion exchange column (purchased from GE Corporation). The specific scheme was as follows: First, the exchange column was rinsed with ultrapure water in a volume of about 100 mL, followed by equilibration with PB solution (pH 5.0) at a flow rate of 2 mL/min. Then 20 mL of the crude extracts of proteins were added to the exchange column at a flow rate of 1 ml/min. After the sample was completely loaded onto the column, the exchange column was rinsed with PB solution (pH 7.0) to remove hybrid protein, and then eluted with PB solution containing 0.5 mol/L NaCl. The proteins at peak value were collected to obtain the proteins of interest.

The proteins were further purified by a hydrophobic chromatography column (purchased from GE Corporation). The specific scheme was as follows: First, the column was rinsed with ultrapure water, and then the hydrophobic column was rinsed with PB solution (pH 7.0) at a flow rate of 2 mL/min. After the column was equilibrated well, the protein sample was loaded onto it. After the sample was completely loaded onto the column, the column was eluted by gradient with PB (pH 7.0) and 1 mol/L NaCl solution for 2 hours. The concentration of NaCl decreases from 1 mol/L to 0.1 mol/L. The proteins were collected at peak value.

The sizes of the resulting P particle protein monomers were identified by reductive SDS-PAGE.

Then the tetracosamers of P protein particles were further isolated and purified using a Superdex 200 molecular sieve (purchased from GE Corporation). The procedures were as follows: The column was rinsed with ultrapure water at a flow rate of 1 mL/min for one volume of the column, and then again with about 120 mL of PB buffer (pH=5). After that, 2 mL of protein extraction solution was added to the column and washed with PB buffer at a flow rate of 1 mL/min. The proteins at peak value were collected to obtain the tetracosamers of P particle proteins. Three proteins were tested for their polymer structures by native polyacrylamide gel electrophoresis. As shown in FIG. 2C, the protein bands are above 250 KDa, which indicates that recombinant P proteins can self-assemble into dodecamers and tetracosamers of P protein particles in vitro and maintain equilibrium; and the proteins can remain their polymer form after being purified. Electron microscopy analysis of purified PP-3C protein particles showed that PP-3C mostly formed large particle aggregates (as shown in FIG. 4).

### Example 3: Preparation of a Lysine-Modified Norovirus P Protein (PP-3K)

In this example, three point mutations were performed using the norovirus P protein plasmid (which was obtained by constructing the PP gene in the pET26b-PP plasmid provided in the Chinese patent application for invention 2015104155561 into pET28a by homologous recombination method) as a template, a lysine is inserted into each of the three loops loop structure of the norovirus P particle, and the protein was expressed and purified.

3.1 Construction of pET28a-PP Plasmid

The PP genes in pET 26b-PP plasmid were enriched by PCR method. The specific method was as follows:

SEQ ID NO: 1361 (forward):
AACTTTAAGAAGGAGATATACCATGGGCAAGCCCTTCTCGGTCCCTA,
and
SEQ ID NO: 1362 (reverse):
CGGATCTCAGTGGTGGTGGTGGTGGTGCTCGAGTTACAAGGCTCTGCG
ACGACCGGC;

the pET26b-P protein plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 50 μL of PCR products. The products were subjected to agarose gel electrophoresis, and the fragments of interest were recovered using gel recovery kit (purchased from Tiangen Biotech Co., Ltd.).

1 μL of BamHI enzyme and 1 μL of XhoI enzyme (purchased from Takara Corporation) and 5 μL of enzyme digestion buffer (purchased from Takara Corporation) were added respectively to 2 μg of pET28a vector plasmid (purchased from Novagen Corporation), and finally sterile water was added to the system to reach a final volume of 50 μL. Digestion was carried out at 37° C. for 2 hours. The products were subjected to agarose gel electrophoresis, and the vector fragments were recovered using gel recovery kit (purchased from Tiangen Biotech Co., Ltd.) to obtain a linear plasmid vector.

The above double enzyme-digested vector fragment and fragment of interest (with a molar ratio of 1:3, and a total volume of 5 μL) were mixed, and 5 μL of homologous recombination mix (purchased from Taihe Corporation) was added. The ligation was carried out at 25° C. for 15 min. 10 μL of the ligated products were added to DH10B competent cells (purchased from Taihe Corporation), and placed on ice for 30 min. After that, the cells were subjected to heat shock at 42° C. for 30 s, placed on ice for 2 min. and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The recombinant clones were obtained, and the sequence was verified by sequencing. A pET28a-PP plasmid that can express PP protein was obtained, as shown in FIG. 1C.

### 3.2 Construction of PP-3K Plasmid

Three rounds of site-directed mutagenesis were performed by site-directed mutation method using the pET28a-PP plasmid which has already been constructed. Respectively, 5′ CGCTGGAACACAAA3′ in loop1 was mutated to 5′ CGCTGGAAAGACACAAA3′. The specific method was as follows:

SEQ ID NO: 1340 (forward):
GACGTCACACACATCGCTGGAAAGACACAAAACTACACCATGAAC,
and
SEQ ID NO: 1341 (reverse):
GTTCATGGTGTAGTTTTGTGTCTTTCCAGCGATGTGTGTGACGTC;

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min, and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing.

The PCR product obtained by the first round of site-directed mutation was subjected to a second round of site-directed mutation. 5′ ACCTCAAACGAT 3′ in loop2 was mutated to 5′ ACCTCAAAGAACGAT3′. The specific method was as follows:

SEQ ID NO: 1342 (forward):
CAATTCAGCACAGACACCTCAAAGAACGATTTCGAGACTGGCCAG,
and
SEQ ID NO: 1343 (reverse):
CTGGCCAGTCTCGAAATCGTTCTTTGAGGTGTCTGTGCTGAATTG;

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min, and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing.

The PCR product obtained by the second round of site-directed mutation was subjected to a third round of site-directed mutation. 5′ GACGGCAGCACC 3′ in loop3 was mutated to 5′ GACGGCAGCACC3′. The specific method was as follows:

SEQ ID NO: 1344 (forward):
GTGGGTGTCGTTCAAGACGGCAAGAGCACCACTCACCAGAACGAA,
and
SEQ ID NO: 1345 (reverse):
TTCGTTCTGGTGAGTGGTGCTCTTGCCGTCTTGAACGACACCCAC;

the entire plasmid was subjected to PCR reaction, wherein the PCR reaction system was a KOD-Plus DNA polymerase system (purchased from TOYOBO Corporation), and the total volume of the reaction system was 50 μL (5 μL buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 20 μL of PCR products. 1 μL of DpnI enzyme (purchased from NEB Corporation) was added to the PCR products. Digestion was carried out at 37° C. for 1 h. 10 μL of the digested products were added to Tran1-Blue competent cells (purchased from Beijing TransGen Biotech, Ltd.), and placed on ice for 30 min. After that, the cells were subjected to heat shock at 42° C. for 45 s, placed on ice for 2 min. and then were added to 600 μL of fluid LB medium without resistance, and recovered at 200 rpm at 37° C. for 1 h. The culture broth was plated on a LB solid culture plate containing kanamycin (15 μg/mL), and were placed upside down at 37° C. overnight. The mutant plasmid clones were obtained, and the sequence was verified by sequencing (the sequence of the PP-3K gene is represented by SEQ ID NO: 1360). The pET28a-P protein-3K plasmid is shown as FIG. 1D.

### 3.3 Expression of the Lysine-Modified Norovirus P Protein

1 μL of the recombinant plasmids prepared in the above examples were respectively added to 100 μL of E. coli BL21 competent cells (purchased from TransGen Corporation), ice-bathed for 30 min, heat shocked for 90 s in a water-bath at 42° C., and then ice-bathed for 2 min. 600 UL of LB medium was added to the mixture, and cultured at 180 rpm/min at 37° C. for 1 h. The mixture was coated evenly on a LB solid medium containing kanamycin (15 μg/mL) resistance and cultured at 37° C. for 24 h to obtain strains that can stably express recombinant proteins. A growing colony was picked and inoculated into 20 mL of LB medium. The mixture was cultured at 220 rpm at 37° C. When the OD value of the culture mixture reached 1.0, induction by Isopropyl β-D-Thiogalactoside (IPTG at a final concentration of 0.5 mmol/L) was carried out at 220 rpm at 16° C. overnight. After the induction, the culture broth was centrifuged at 4,000 rpm for 20 min. The supernatant was discarded, and the bacterial precipitates were resuspended with PBS. Centrifugation was conducted again at 4,000 rpm for 20 min and the supernatant was discarded to obtain the bacterial precipitates containing proteins of interest.

### 3.4 Purification of the Lysine-Modified Norovirus P Protein

The bacterial precipitates obtained in 3.3 were resuspended by adding 20 ml of protein buffer (pH8.0, containing 50 mM Tris and 300 mM KCl). The bacteria were lysed by sonication on ice for 30 min. The mixture was centrifuged at 12,000 rpm at 4° C. for 30 min. Subsequently, the supernatant was taken and allowed to pass through 0.45 μm filter membrane to obtain crude extracts of proteins.

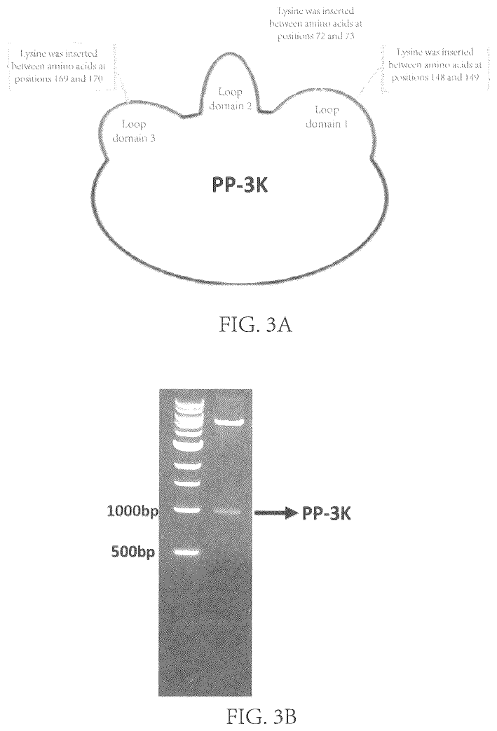

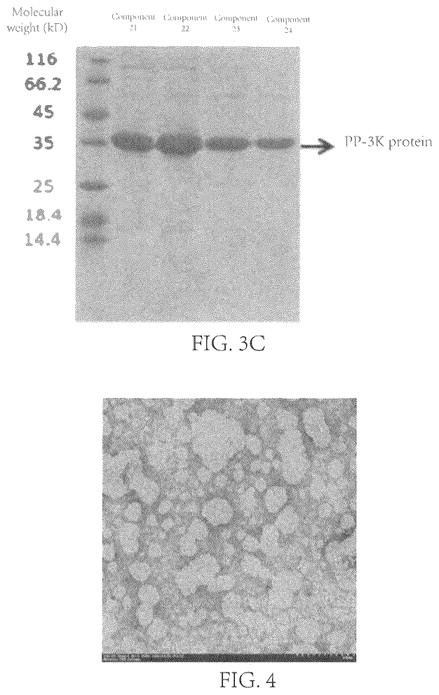

The structure of the recombinant PP-3K particle is shown as FIG. 3A.

The crude extracts of proteins were purified using an anion exchange column (purchased from GE Corporation). The specific scheme was as follows: First, the exchange column was rinsed with ultrapure water in a volume of about 100 mL, followed by equilibration with PB solution (pH 5.0) at a flow rate of 2 mL/min. Then 20 mL of the crude extracts of proteins were added to the exchange column at a flow rate of 1 ml/min. After the sample was completely loaded onto the column, the exchange column was rinsed with PB solution (pH 7.0) to remove hybrid protein, and then eluted with PB solution containing 0.5 mol/L NaCl. The proteins at peak value were collected to obtain the proteins of interest.

The proteins were further purified by a hydrophobic chromatography column (purchased from GE Corporation). The specific scheme was as follows: First, the column was rinsed with ultrapure water, and then the hydrophobic column was rinsed with PB solution (pH 7.0) at a flow rate of 2 mL/min. After the column was equilibrated well, the protein sample was loaded onto it. After the sample was completely loaded onto the column, the column was eluted by gradient with PB (pH 7.0) and 1 mol/L NaCl solution for 2 hours. The concentration of NaCl decreases from 1 mol/L to 0.1 mol/L. The proteins were collected at peak value.

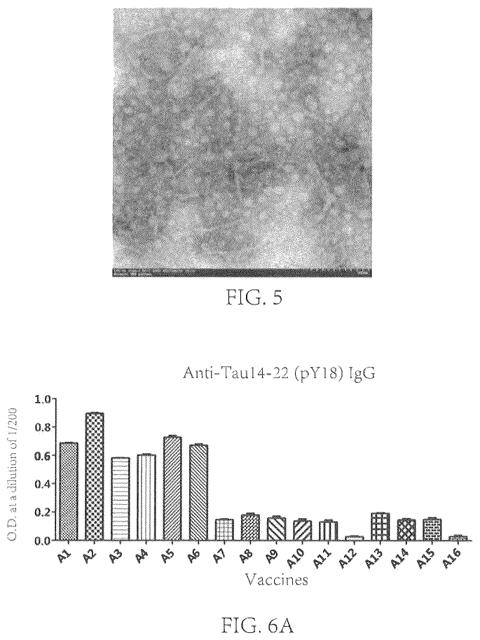

The sizes of P particle protein monomers were identified by reductive SDS-PAGE. Then the tetracosamers of P protein particles were further isolated and purified using a Superdex 200 molecular sieve (purchased from GE Corporation). The procedures were as follows: The column was rinsed with ultrapure water at a flow rate of 1 mL/min for one volume of the column, and then again with about 120 mL of PB buffer (pH=5). After that, 2 mL of protein extraction solution was added to the column and washed with PB buffer at a flow rate of 1 mL/min. The proteins at peak value were collected to obtain the tetracosamers of P particle proteins. Three proteins were tested for their polymer structures by native polyacrylamide gel electrophoresis, as shown in FIG. 2C. Protein bands are above 250 KDa, which indicates that recombinant P proteins can self-assemble into dodecamers and tetracosamers of P protein particles in vitro and maintain equilibrium; and the proteins can remain their polymer form after being purified. Electron microscopy analysis of purified PP-3K protein particles showed that PP-3K mostly formed large particle aggregates (as shown in FIG. 5).

### Example 4: Coupling Reaction of PP-3C with a Phosphorylated Polypeptide Antigen Vaccine

Example 4-1: The purified PP-3C was subjected to a buffer-exchange treatment with a desalting column from GE Corporation to change the buffer system of the protein to a 0.1 M ammonium bicarbonate solution (pH=7.5). The PP-3C after the buffer-exchange treatment was quantified, and was diluted to a concentration of 0.3 mg/mL. In the ratio of the molar amount of protein to the molar amount of polypeptide of 1:30, each of the phosphorylated polypeptide antigen vaccines in lyophilized form prepared in Example 1 was added and mixed slowly. The mixed system was incubated at 2° C. to 8° C. with slow shaking for 48 hours. The resulting product was concentrated by ultrafiltration to remove unbound polypeptides, and the buffer system was changed to a PBS buffer system.

Example 4-2: The purified PP-3C was subjected to a buffer-exchange treatment with a desalting column from GE Corporation to change the buffer system of the protein to a 1 M ammonium bicarbonate solution (pH=8.0). The PP-3C after the buffer-exchange treatment was quantified, and was diluted to a concentration of 0.5 mg/mL. In the ratio of the molar amount of protein to the molar amount of polypeptide of 1:50, each of the phosphorylated polypeptide antigen vaccines in lyophilized form prepared in Example 1 was added and mixed slowly. The mixed system was incubated at 2° C. to 8° C. with slow shaking for 48 hours. The resulting product was concentrated by ultrafiltration to remove unbound polypeptides, and the buffer system was changed to a PBS buffer system.

Example 4-3: The purified PP-3C was subjected to a buffer-exchange treatment with a desalting column from GE Corporation to change the buffer system of the protein to a 0.1 M ammonium bicarbonate solution (pH=7.8). The PP-3C after the buffer-exchange treatment was quantified, and was diluted to a concentration of 0.3 mg/mL. In the ratio of the molar amount of protein to the molar amount of polypeptide of 1:100, each of the phosphorylated polypeptide antigen vaccines in lyophilized form prepared in Example 1 was added and mixed slowly. The mixed system was incubated at 2° C. to 8° C. with slow shaking for 48 hours. The resulting product was concentrated by ultrafiltration to remove unbound polypeptides, and the buffer system was changed to a PBS buffer system.

Example 4-4: The purified PP-3C was subjected to a buffer-exchange treatment with a desalting column from GE Corporation to change the buffer system of the protein to a 0.1 M ammonium bicarbonate solution (pH=8.8). The PP-3C after the buffer-exchange treatment was quantified, and was diluted to a concentration of 0.3 mg/mL. In the ratio of the molar amount of protein to the molar amount of polypeptide of 1:30, each of the phosphorylated polypeptide antigen vaccines in lyophilized form prepared in Example 1 was added and mixed slowly. The mixed system was incubated at 2° C. to 8° C. with slow shaking for 72 hours. The resulting product was concentrated by ultrafiltration to remove unbound polypeptides, and the buffer system was changed to a PBS buffer system.

### Example 5: Coupling Reaction of PP-3K with a Phosphorylated Polypeptide Antigen Vaccine

Example 5-1: The purified PP-3K solution was quantified by BCA and the pH was adjusted to 7.2. This protein solution was mixed with a 1 mg/mL sulfo-maleimide (sulfo-SMCC) solution in the ratio of the molar amount of SMCC to the molar amount of PP-3K of 5:1, and the mixture was reacted in a water bath at 25° C. for 30 min. The mixture was subjected to a desalting treatment with a desalting column from GE Corporation, the system was changed to a PBS solution (pH=7.2-7.4), and unbound sulfo-SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptide of 1:5, and mixed slowly. Then, the reaction system was reacted in a water bath at 25° C. for 30 min. The reaction product was collected, and the mixture was subjected to a desalting treatment with a desalting column from GE Corporation, the system was changed to a PBS solution (pH=7.2-7.4), and unbound phosphorylated polypeptides were removed.

Example 5-2: The purified PP-3K solution was quantified by BCA. This protein solution was mixed with a 1 mg/mL sulfo-maleimide (sulfo-SMCC) solution in the ratio of the molar amount of SMCC to the molar amount of PP-3K of 20:1, and the mixture was reacted in a water bath at 25° C. for 30 min. The mixture was subjected to a desalting treatment with a desalting column from GE Corporation, the system was changed to a PB solution (pH=8.5), and unbound sulfo-SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptides of 1:30, and mixed slowly. Then, the reaction system was reacted in a water bath at 25° C. for 30 min. The reaction product was collected, and the mixture was subjected to a desalting treatment with a desalting column from GE Corporation, the system was changed to a PB solution (pH=8.5), and unbound phosphorylated polypeptides were removed.

Example 5-3: The purified PP-3K solution was quantified by BCA. This protein solution was mixed with a 1 mg/mL sulfo-maleimide (sulfo-SMCC) solution in the ratio of the molar amount of SMCC to the molar amount of PP-3K of 20:1, and the mixture was reacted in a water bath at 25° C. for 30 min. The mixture was subjected to a desalting treatment with a dialysis bag having a molecular weight cut-off of 20 kd (the dialysis volume ratio is 1,000:1), and was dialyzed overnight at 2° C. to 8° C., the system was changed to a PB solution (pH=7.0), and unbound sulfo-SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptides of 1:50, and mixed slowly. Then, the reaction system was reacted in a water bath at 25° C. for 30 min. The mixture was subjected to a desalting treatment with a dialysis bag having a molecular weight cut-off of 20 kDa (the dialysis volume ratio is 1,000:1), and was dialyzed overnight at 2° C. to 8° C., the system was changed to a PB solution (pH=7.0), and unbound phosphorylated polypeptides were removed.

Example 5-4: The purified PP-3K solution was quantified by BCA. This protein solution was mixed with a 1 mg/mL sulfo-maleimide (sulfo-SMCC) solution in the ratio of the molar amount of SMCC to the molar amount of PP-3K of 20:1, and the mixture was reacted with slow shaking overnight at 2° C. to 8° C. The mixture was subjected to a desalting treatment with a dialysis bag having a molecular weight cut-off of 20 kd (the dialysis volume ratio is 1,000:1), and was dialyzed overnight at 2° C. to 8° C., the system was changed to a PBS solution (pH=7.2-7.4), and unbound sulfo-SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptides of 1:50, and mixed slowly. Then, the reaction system was reacted with slow shaking overnight at 2° C. to 8° C. The mixture was subjected to a desalting treatment with a dialysis bag having a molecular weight cut-off of 20 kd (the dialysis volume ratio is 1,000:1), and was dialyzed overnight at 2° C. to 8° C., the system was changed to a PBS solution (pH=7.2-7.4), and unbound phosphorylated polypeptides were removed.

Example 5-5: The purified PP-3K solution was quantified by BCA. This protein solution was mixed with a 20 mg/ml dimethylformamide (DMF) solution of maleimide (SMCC) in the ratio of the molar amount of SMCC to the molar amount of PP-3K of 10:1, and the mixture was reacted in a water bath at 25° C. for 30 min. The mixture was subjected to a desalting treatment with a desalting column from GE Corporation, the system was changed to a PBS solution (pH=7.2-7.4), and unbound SMCC was removed. The reaction product was taken and each of the lyophilized products of the phosphorylated polypeptide antigen vaccines prepared in Example 1 was added to the system in a ratio of the molar amount of PP-3K to the molar amount of phosphorylated polypeptides of 1:30, and mixed slowly. Then, the reaction system was reacted in a water bath at 25° C. for 30 min. The reaction product was collected, and the mixture was subjected to a desalting treatment with a desalting column from GE Corporation, the system was changed to a PBS solution (pH=7.2), and unbound phosphorylated polypeptides were removed.

Example 5-6: The purified PP-3K solution was quantified by BCA. This protein solution was mixed with a 20 mg/ml dimethylformamide (DMF) solution of maleimide (SMCC) in the ratio of the molar amount of SMCC to the molar amount of PP-3K of 30:1, and the mixture was reacted with slow shaking overnight at 2° C. to 8° C. The mixture was subjected to a desalting treatment with a desalting column from GE Corporation, the system was changed to a PBS solution (pH=7.2-7.4), and unbound SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptides of 1:50, and mixed slowly. The mixture was reacted with slow shaking overnight at 2° C. to 8° C. The reaction product was collected, and the mixture was subjected to a desalting treatment with a desalting column from GE Corporation, the system was changed to a PBS solution (pH=7.2), and unbound phosphorylated polypeptides were removed.

Example 5-7: The purified PP-3K solution was quantified by BCA. This protein solution was mixed with a 20 mg/ml dimethylformamide (DMF) solution of maleimide (SMCC) in the ratio of the molar amount of SMCC to the molar amount of PP-3K of 5:1, and the mixture was reacted in a water bath at 25° C. for 30 min. The mixture was subjected to a desalting treatment with a dialysis bag having a molecular weight cut-off of 20 kDa (the dialysis volume ratio is 1,000:1), and was dialyzed overnight at 2° C. to 8° C., the system was changed to a PBS solution (pH=7.2-7.4), and unbound SMCC was removed. The reaction product was taken and each of the lyophilized products of the phosphorylated polypeptide antigen vaccines prepared in Example 1 was added to the system in the ratio of the molar amount of PP-3K to the molar amount of phosphorylated polypeptides of 1:100, and mixed slowly. The mixture was reacted with slow shaking overnight at 2° C. to 8° C. The reaction product was collected, and the mixture was subjected to a desalting treatment with a dialysis bag having a molecular weight cut-off of 20 kDa (the dialysis volume ratio is 1,000:1), and was dialyzed overnight at 2° C. to 8° C., the system was changed to a PBS solution (pH=7.2), and unbound phosphorylated polypeptides were removed.

### Example 6: Determination of the Ligation Efficiency of the Phosphorylated Polypeptide Antigen Product Coupled with PP-3C

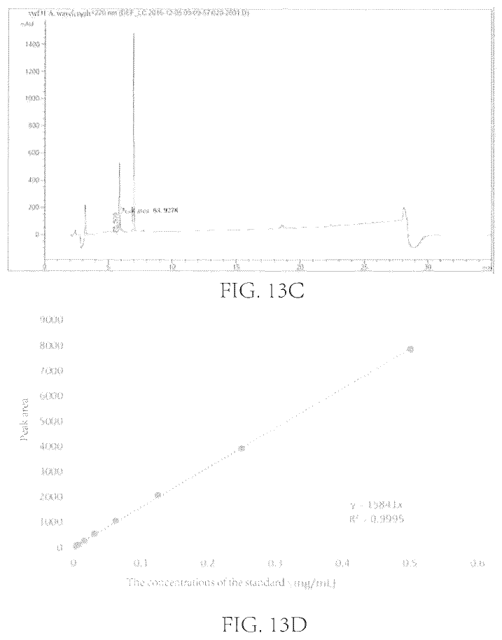

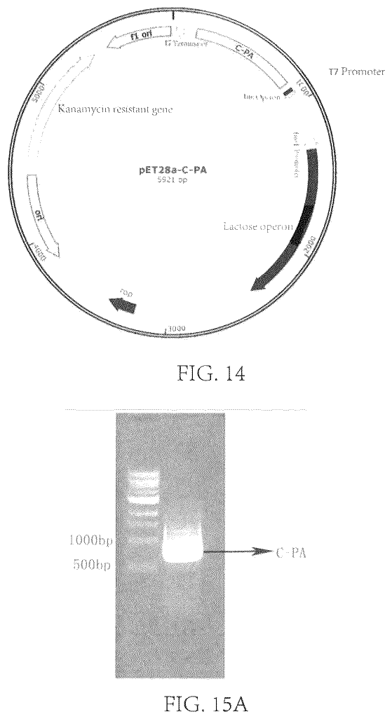

A10 mM dithiothreitol solution was added to the phosphorylated polypeptide antigen product coupled with PP-3C at a volume ratio of 1:1, and mixed homogeneously, and the mixture was left to stand at room temperature and react for 16 hours. The reaction product was centrifuged at 16,000 g at 4° C. for 15 min. The supernatants were taken for HPLC determination. Using 0.5 mg/mL, 0.25 mg/mL, 0.125 mg/mL, 0.0625 mg/mL, 0.03125 mg/mL, 0.03125 mg/mL, and 0.015625 mg/mL phosphorylated polypeptide dissolved in 10 mM of a dithiothreitol solution as standard, a standard curve of the concentration of the standard versus the peak area of the phosphorylated polypeptide was plotted for quantitatively analyzing the concentration of the test sample (the results are shown in FIGS. 13A, 13B and 13D).

### Example 7: Determination of the Ligation Efficiency of the Phosphorylated Polypeptide Antigen Product Coupled with PP-3K

The phosphorylated polypeptide antigen product coupled with PP-3K was subjected to a mass spectrometric analysis, and the concentration of the test sample was quantitatively analyzed using the phosphorylated polypeptide dissolved in PBS as standard (the results are not shown).

### Example 8: Analysis of the Immunogenicity of Phosphorylated Polypeptide Antigen Vaccines in Wild Mice

The phosphorylated polypeptide antigens with sequences as represented by SEQ ID NO: 201, SEQ ID NO: 225, SEQ ID NO: 306, SEQ ID NO: 387, SEQ ID NO: 468, SEQ ID NO: 558, SEQ ID NO: 567, SEQ ID NO: 769, SEQ ID NO: 784, SEQ ID NO: 875, SEQ ID NO: 1020, SEQ ID NO: 1101, SEQ ID NO: 1182, SEQ ID NO: 1272, SEQ ID NO: 1313 and SEQ ID NO: 1330 were dissolved to 1 mg/mL, and mixed with a complete Freund's adjuvant or an incomplete Freund's adjuvant at a volume ratio of 1:1 to form a water-in-oil emulsion so as to prepare vaccines A1 to A16. Each wild mouse was injected intramuscularly with 50 μL. Each wild mouse was vaccinated four times at two-week intervals using complete Freund's adjuvant for the first immunization and incomplete Freund's adjuvant for the second, third and fourth immunizations. Two weeks after the fourth injection, the mice were subjected to blood sampling to obtain mouse serum for ELISA analysis.

The polypeptide (whose sequence is represented by SEQ ID NO: 1346) which was coupled with BSA and phosphorylated at a position corresponding to amino acid 18 of full-length Tau protein was used as the coating antigen to detect the antiserum titer against a phosphorylation Y18 site; the phosphorylatd polypeptide (whose sequence is represented by SEQ ID NO: 1347) which was coupled with BSA and phosphorylated at positions corresponding to amino acids 202 and 205 of full-length Tau protein was used as the coating antigen to detect the antiserum titer against phosphorylation S202 and T205 sites; the phosphorylatd polypeptide (whose sequence is represented by SEQ ID NO: 1348) which was coupled with BSA and phosphorylated at a position corresponding to amino acid 212/214 of full-length Tau protein was used as the coating antigen to detect the antiserum titer against phosphorylation T212 and S214 sites; the phosphorylatd polypeptide (whose sequence is represented by SEQ ID NO: 1349) which was coupled with BSA and phosphorylated at a position corresponding to amino acid 231/235 of full-length Tau protein was used as the coating antigen to detect the antiserum titer against phosphorylation T231 and S235 sites; the phosphorylatd polypeptide (whose sequence is represented by SEQ ID NO: 1350) which was coupled with BSA and phosphorylated at a position corresponded to amino acid 238 of full-length Tau protein was used as the coating antigen to detect the antiserum titer against a phosphorylation S238 site; the phosphorylatd polypeptide (whose sequence is represented by SEQ ID NO: 1351) which was coupled with BSA and phosphorylated at a position corresponding to amino acid 262 of full-length Tau protein was used as the coating antigen to detect the antiserum titer against a phosphorylation S262 site; the phosphorylatd polypeptide (whose sequence is represented by SEQ ID NO: 1352) which was coupled with BSA and phosphorylated at a position corresponding to amino acid 396 of full-length Tau protein was used as the coating antigen to detect the antiserum titer against a phosphorylation S396 site; the phosphorylatd polypeptide (whose sequence is represented by SEQ ID NO: 1353) which was coupled with BSA and phosphorylated at a position corresponding to amino acid 404 of full-length Tau protein was used as the coating antigen to detect the antiserum titer against a phosphorylated S404 site.

The above antigens were respectively dissolved in carbonate buffer with pH=9.5, diluted to 1 μg/mL, and added to an ELISA plate (100 μL/well) at 4° C. overnight. The liquid in the plate was discarded, and after the 96-well plate was washed three times with PBST (300 μL/well), the liquid in the plate was discarded. A3% BSA (PBS) solution was added (200 μL/well), and incubation was carried out at 37° C. for 1 hour. The liquid in the plate was discarded, and after the 96-well plate was washed three times with PBST (300 μL/well), the liquid in the plate was discarded. Mouse serum diluted by gradient with a 1% BSA (PBS) solution was added (100 μL/well), and incubation was carried out at 37° C. for 2 hour. The liquid in the plate was discarded, and after the 96-well plate was washed three times with PBST (300 L/well), the liquid in the plate was discarded. A goat-anti-mouse HRP labled antibody solution diluted with a 1% BSA (PBS) solution was added (100 μL/well), and incubation was carried out at 37° C. for 1 hour. The liquid in the plate was discarded, and after the 96-well plate was washed three times with PBST (300 μL/well), the liquid in the plate was discarded. TMB was added (300 μL/well) and incubation was carried out in the dark at room temperature for 25 min. 1M sulfuric acid aqueous solution was added to terminate the reaction. Absorbance was detected at 450 nm using a microplate reader

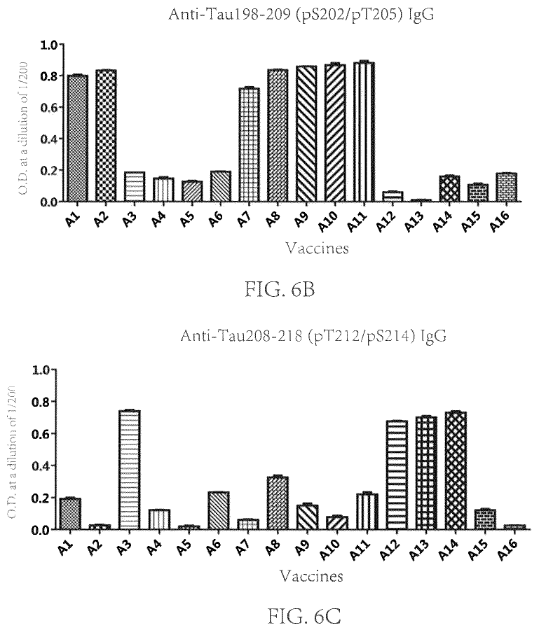

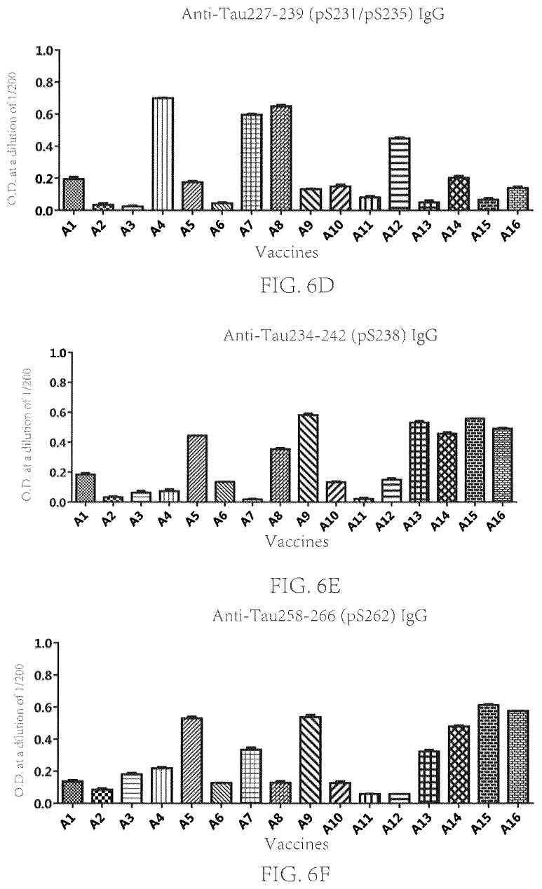

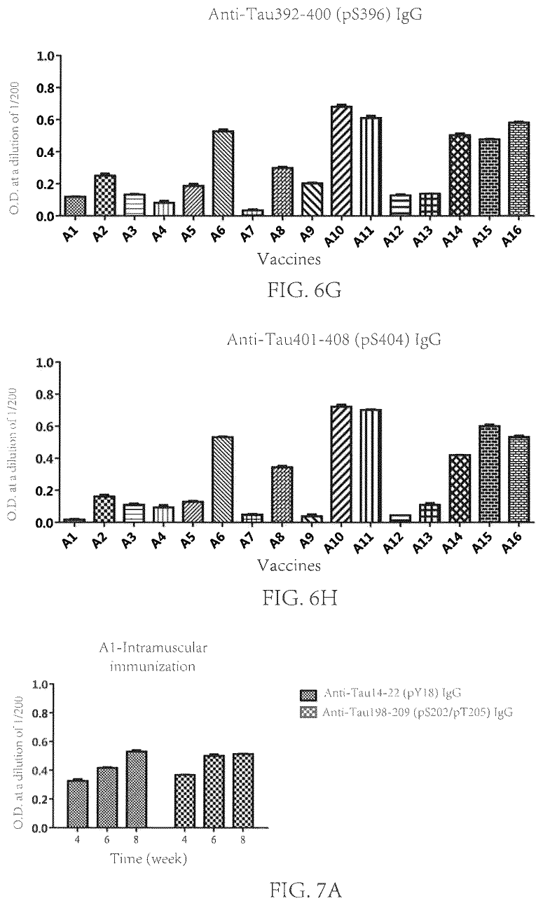

The levels of the antibodies against different antigens in the serum after the fourth immunization by vaccines A1 to A16 are shown in FIG. 6. The results show that all mice in groups of vaccines A1 to A6 can successfully produce antibodies against the pY18 site after immunization, wherein vaccines A2 and A5 can produce high concentrations of antibodies; and vaccines A7 to A16 can hardly produce antibodies against the pY18 site (FIG. 6A). All the groups of mice immunized with vaccines A1 to A2 and A7 to A11 can successfully produce antibodies against the pS202/pT205 site after immunization, wherein vaccines A8 to A11 can produce high concentrations of antibodies; and vaccines A3 to A6 and A12 to A16 can hardly produce antibodies against the pS202/pT205 site (FIG. 6B). All the groups of mice immunized with vaccines A3 and A12 to A14 can successfully produce high concentrations of antibodies against the pT212/pS214 site after immunization, vaccines A6, A8 and A11 can produce low concentrations of antibodies; and vaccines A1, A2, A4, A5, A7, A9 to A11, A15 and A16 can hardly produce antibodies against the pT212/pS214 site (FIG. 6C). All the groups of mice immunized with vaccines A4, A7 to A8 and A12 can successfully produce high concentrations of antibodies against the pS231/pS235 site after immunization, vaccines A1, A5 and A14 can produce low concentrations of antibodies; and vaccines A2, A3, A6, A9 to A11, A13, A15 and A16 can hardly produce antibodies against the pS231/pS235 site (FIG. 6D). All the groups of mice immunized with vaccines A5, A9, A13 and A15 to A16 can successfully produce high concentrations of antibodies against the pS238 site after immunization, vaccines A8 and A14 can also produce relatively high concentrations of antibodies; and vaccines A1 to A4, A6, A7 and A10 to A12 can hardly procude antibodies against the pS238 site (FIG. 6E). All the groups of mice immunized with vaccines A5, A9, A13 and A16 can successfully produce high concentrations of antibodies against the pS262 site after immunization, vaccines A7 and A14 can also produce relatively high concentrations of antibodies; and vaccines A1 to A4, A6, A8 and A10 to A12 can hardly produce antibodies against the pS238 site (FIG. 6F). All the groups of mice immunized with vaccines A6, A10, A11 and A14 to A16 can successfully produce high concentrations of antibodies against the pS396 site after immunization, vaccine A8 can produce low concentrations of antibodies; and vaccines A1 to A5, A7, A9 and A12 to A13 can hardly produce antibodies against the pS396 site (FIG. 6G). All the groups of mice immunized with vaccine A6, A10, A11 and A14 to A16 can successfully produce high concentrations of antibodies against the pS404 site after immunization, vaccine A8 can produce low concentrations of antibodies; and vaccine A1 to A5, A7, A9 and A12 to A13 can hardly produce antibodies against the pS396 site (FIG. 6H). In conclusion, after used for immunizing animals, all the phosphorylated polypeptide vaccines designed in Example 1 can produce antibodies against the corresponding phosphorylation sites and have immunogenicity, and vaccine A8 and vaccine A14 can produce cross-protection responses against other phosphorylation sites; meanwhile, it was found that immunization with relatively long phosphorylated peptides can lead to relatively high concentrations of antibodies.

### Example 9: Evaluation of the Immunogenicity of Phosphorylated Polypeptide Antigens Coupled with the Norovirus P Protein PP-3C in Wild Mice Immunized with the Same by Intramuscular Injection

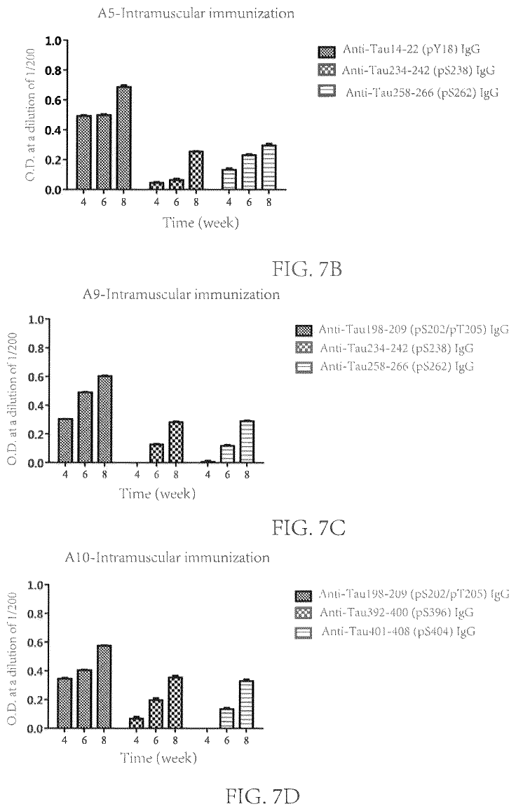

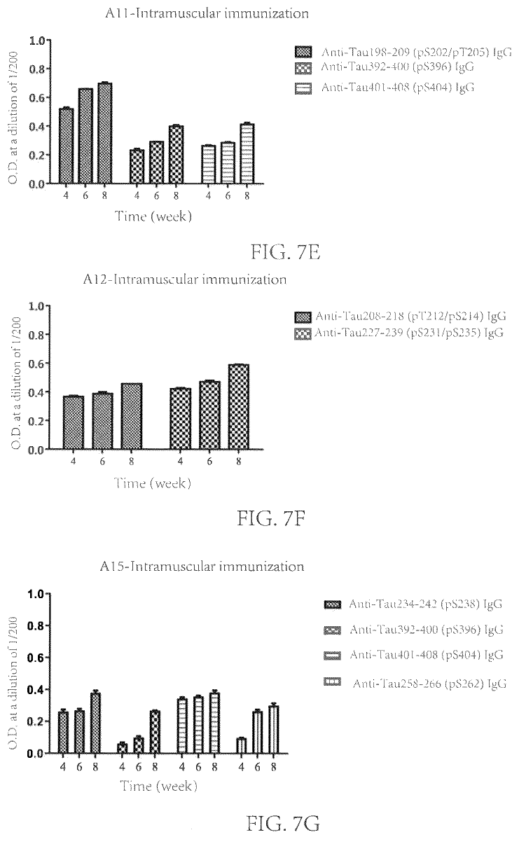

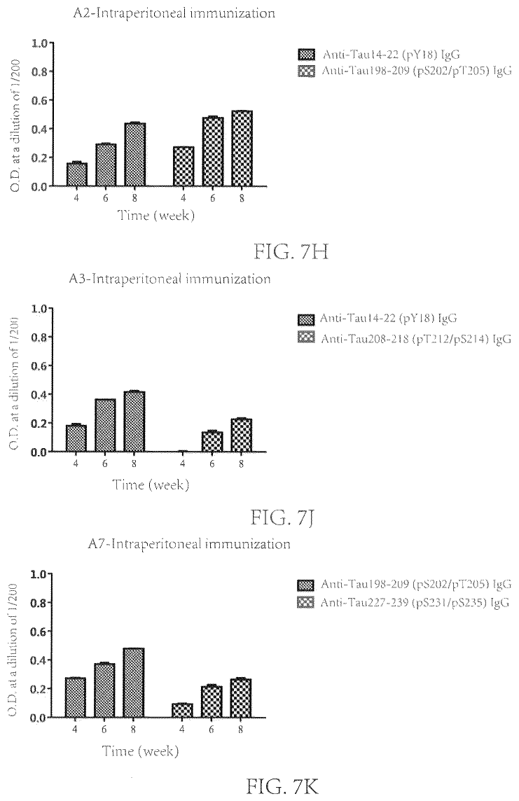

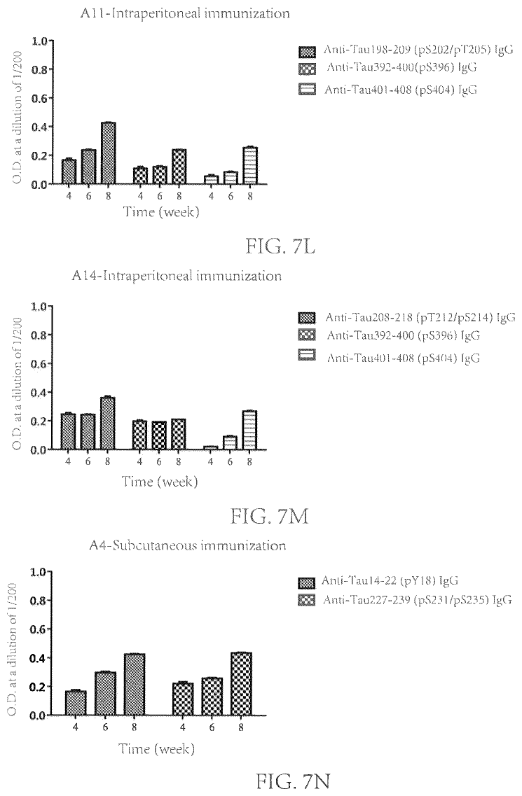

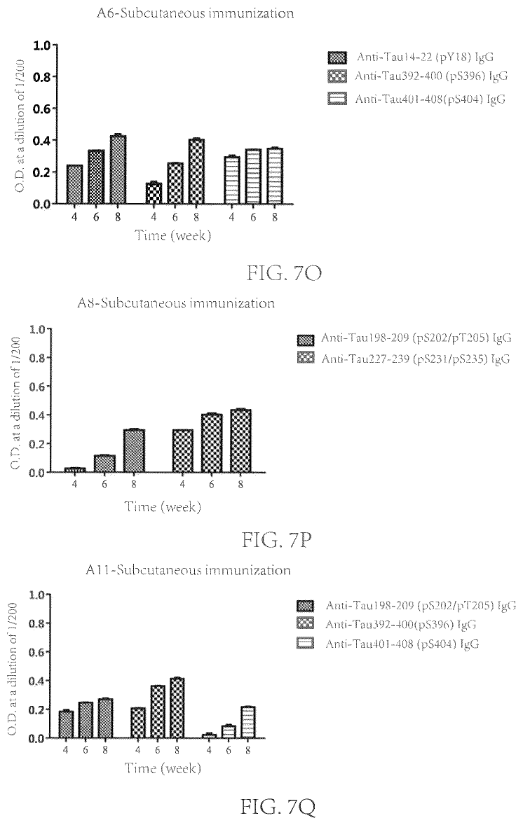

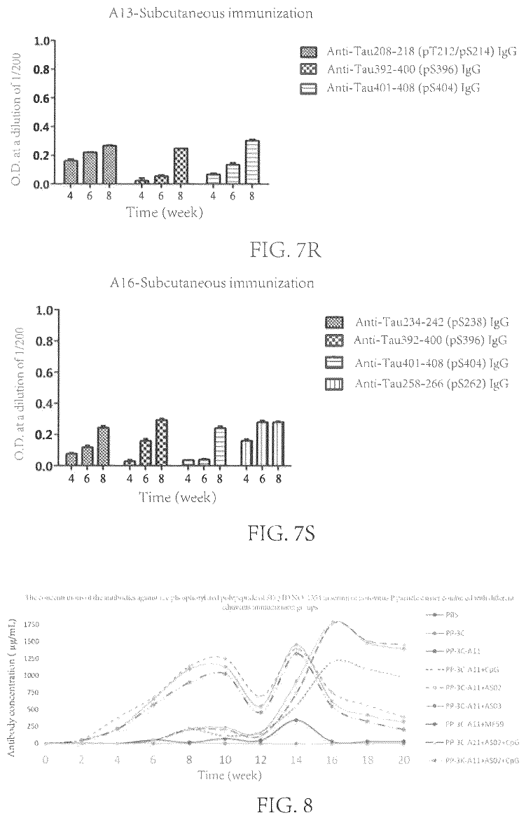

The PP-3C protein was coupled with A1, A5, A9, A10, A11, A12 and A15 respectively to prepare complex vaccines, and wild mice were immunized with the complex vaccines by intramuscular injection. Each group contained 6 mice. The immune dosage of the protein was 25 μg/animal. The immunization was carried out at week 0, week 2, week 4 and week 6. Blood was taken two weeks after every immunization to obtain serum for ELISA assessment. The assessment was performed by the same method as in Example 8. The results are shown in FIGS. 7A-7G. As apparent from the figures, mice immunized with complex vaccine A1 can produce specific antibodies against pY18 and pS202/pT205 (FIG. 7A); mice immunized with complex vaccine A5 can produce specific antibodies against pY18, pS238 and pS262 (FIG. 7B); mice immunized with complex vaccine A9 can produce specific antibodies against pS202/pT205, pS238 and pS262 (FIG. 7C); mice immunized with complex vaccine A9 can produce specific antibodies against pS202/pT205, pS396 and pS404 (FIG. 7D); mice immunized with complex vaccine A11 can produce specific antibodies against pS202/pT205, pS396 and pS404 (FIG. 7E); mice immunized with complex vaccine A12 can produce specific antibodies against pT212/pS214 and pS231/pS235 (FIG. 7F); and mice immunized with complex vaccine A15 can produce specific antibodies against pS238, pS262, pS396 and pS404 (FIG. 7G). Thus, it can be seen that the concentration of the specific antibodies in the serum increased with each intramuscular immunization with the complex vaccine in each group, and after the fourth immunization high concentrations of antibodies were produced, wherein pY18 and pS202/pT205 epitopes are prone to leading to high concentrations of specific antibodies. Compared with other immunization routes, intramuscular immunization generally can lead to higher concentrations of antibodies.

### Example 10: Evaluation of the Immunogenicity of Phosphorylated Polypeptide Antigens Coupled with the Norovirus P Protein PP-3C in Wild Mice Immunized with the Same by Intraperitoneal Injection

The PP-3C protein was coupled with A2, A3, A7, A11 and A14 respectively to prepare complex vaccines, and wild mice were immunized with the complex vaccines by intraperitoneal injection. Each group contained 6 mice. The immune dosage of the protein was 25 μg/animal. The immunization was carried out at week 0, week 2, week 4 and week 6. Blood was taken two weeks after every immunization to obtain serum for ELISA assessment. The assessment was performed by the same method as in Example 10. The results were shown in FIGS. 7H-7M. As apparent from the figures, mice immunized with complex vaccine A2 can produce specific antibodies against pY18 and pS202/pT205 (FIG. 7H); mice immunized with complex vaccine A3 can produce specific antibodies against pY18 and pT212/pS214 (FIG. 7J); mice immunized with complex vaccine A7 can produce specific antibodies against pS202/pT205 and pS231/pS235 (FIG. 7K); mice immunized with complex vaccine A11 can produce specific antibodies against pS202/pT205, pS396 and pS404 (FIG. 7L); mice immunized with complex vaccine A14 can produce specific antibodies against pT212/pS214, pS396 and pS404 (FIG. 7M). Thus, it can be seen that the concentration of the specific antibodies in the serum increased with each intraperitoneal immunization with the complex vaccine in each group, and after the fourth immunization high concentrations of antibodies were produced.

### Example 11: Evaluation of the Immunogenicity of Phosphorylated Polypeptide Antigens Coupled with the Norovirus P Protein PP-3C in Wild Mice Immunized with the Same by Subcutaneous Injection

The PP-3C protein was coupled with A4, A6, A8, A11, A13 and A16 respectively to prepare complex vaccines, and wild mice were immunized with the complex vaccines by subcutaneous injection. Each group contained 6 mice. The immune dosage of the protein was 25 μg/animal. The immunization was carried out at week 0, week 2, week 4 and week 6. Blood was taken two weeks after every immunization to obtain serum for ELISA assessment. The assessment was performed by the same method as in Example 10. The results were shown in FIGS. 7N-7S. As apparent from the figures, mice immunized with complex vaccine A4 can produce specific antibodies against pY18 and pS231/pS235 (FIG. 7N); mice immunized with complex vaccine A6 can produce specific antibodies against pY18, pS396 and pS404 (FIG. 7O); mice immunized with complex vaccine A8 can produce specific antibodies against pS202/pT205 and pS231/pS235 (FIG. 7P); mice immunized with complex vaccine A11 can produce specific antibodies against pS202/pT205, pS396 and pS404 (FIG. 7Q); mice immunized with complex vaccine A13 can produce specific antibodies against pT212/pS214, pS396 and pS404 (FIG. 7R); and mice immunized with complex vaccine A16 can produce specific antibodies against pS238, pS262, pS396 and pS404 (FIG. 7S). Thus, it can be seen that the concentration of the specific antibodies in the serum increased with each subcutaneous immunization with the complex vaccine in each group, and after the fourth immunization high concentrations of antibodies were produced.

### Example 12: Evaluation of the Immunogenicity of Phosphorylated Polypeptide Antigens Coupled with a Norovirus P Protein in Wild Mice

Take vaccine A11 as an example. Wild mice were intramuscularly immunized with A11 coupled with PP-3C or PP-3K and combined with CpG, AS02, AS03, MF59 and AS02+CpG adjuvants respectively. Each group contained 6 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1354) coupled with BSA was used as the coating antigen, and was phosphorylated at sites corresponding to amino acids 202, 205, 396 and 404 of full-length Tau protein. A standard curve was plotted with the concentration of the AT8 antibody (Thermo scientific MN1020) diluted by gradient versus the O.D. value to calibrate the concentration of the antibodies in the mouse serum. The change in antibody concentration in each group is shown in FIG. 8. As can be seen from FIG. 8, vaccine A11 coupled with a norovirus P protein and combined with different adjuvants could elicit relatively high immune responses in wild mice, wherein the vaccine in combination with AS02, AS03 and MF59 enabled gradual production of relatively high concentrations of antibodies through four consecutive immunizations, and failed to stimulate and elicit a relatively high immune response in one immunization after an interval of 6 weeks; the vaccine combined with CpG adjuvant and AS02+CpG adjuvant enabled gradual production of relatively low concentrations of antibodies through four consecutive immunizations, and stimulated the production of a relatively high concentration of antibodies in one immunization after an interval of 6 weeks, and the antibody content could maintain a high level for a long time. The production of antibodies in mice immunized with vaccine A11 coupled with PP-3C was basically the same with that in mice immunized with vaccine A11 coupled with PP-3K. Immunization of wild mice with the other phosphorylated polypeptide vaccines produced similar effects (the results are not shown).

### Example 13: Evaluation of the Immunogenicity of Phosphorylated Polypeptide Antigen A5 Coupled with a Norovirus P Protein in Wild Mice

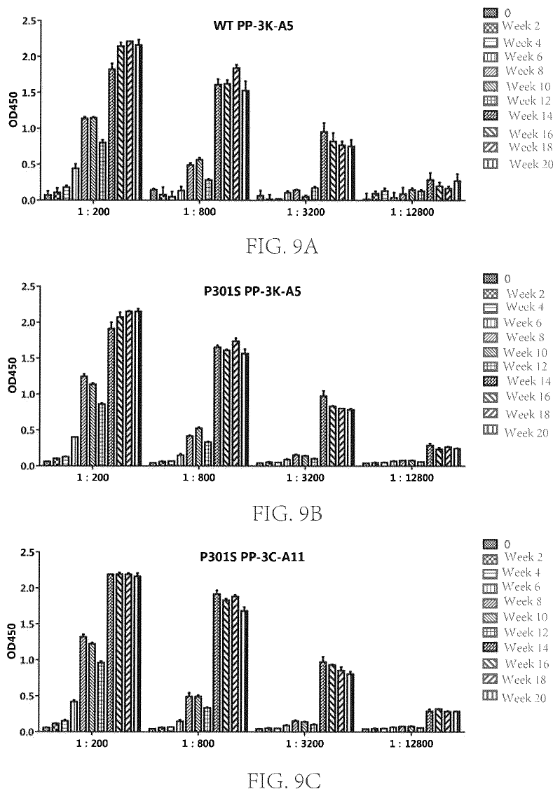

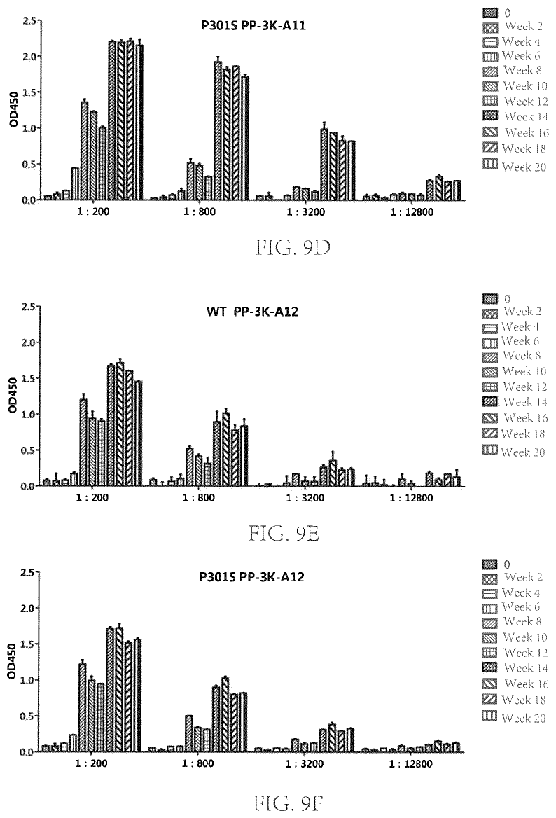

Wild mice were intramuscularly immunized with the vaccine prepared by combining phosphorylated polypeptide antigen A5 coupled with PP-3K with AS02+CpG adjuvant. Each group contained 6 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1355) coupled with BSA was used as the coating antigen, and was phosphorylated at sites corresponding to amino acids 18, 238 and 262 of full-length Tau protein. The change in the concentration of antibodies in the mouse serum is shown in FIG. 9A. The results showed that intramuscular immunization with the PP-3K-A5 complex vaccine in combiantion with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 14: Evaluation of the Immunogenicity of Phosphorylated Polypeptide Antigen A5 Coupled with a Norovirus P Protein in P301S Transgenic Mice

P301S transgenic mice were intramuscularly immunized with the vaccine prepared by combining phosphorylated polypeptide antigen A5 coupled with PP-3K with AS02+CpG adjuvant. Each group contained 8 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1355) coupled with BSA was used as the coating antigen, and was phosphorylated at sites corresponding to amino acids 18, 238 and 262 of full-length Tau protein. The change in the concentration of antibodies in the mouse serum is shown in FIG. 9B. The results showed that intramuscular immunization with the PP-3K-A5 complex vaccine in combination with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 15: Evaluation of the Immunogenicity of Phosphorylated Polypeptide Antigen A11 Coupled with a Norovirus P Protein in P301S Transgenic Mice

P301S transgenic mice were intramuscularly immunized with the vaccine prepared by combining phosphorylated polypeptide antigen A11 coupled with PP-3C with AS02+CpG adjuvant. Each group contained 12 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1354) coupled with BSA was used as the coating antigen, and was phosphorylated at sites corresponding to amino acids 202, 205, 396 and 404 of full-length Tau protein. The change in the concentration of antibodies in the mouse serum is shown in FIG. 9C. The results showed that intramuscular immunization with the PP-3K-A11 complex vaccine in combination with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 16: Evaluation of the Immunogenicity of Phosphorylated Polypeptide Antigen A11 Coupled with a Norovirus P Protein in P301S Transgenic Mice

P301S transgenic mice were intramuscularly immunized with the vaccine prepared by combining phosphorylated polypeptide antigen A11 coupled with PP-3K with AS02+CpG adjuvant. Each group contained 12 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1354) coupled with BSA was used as the coating antigen, and was phosphorylated at sites corresponding to amino acids 202, 205, 396 and 404 of full-length Tau protein. The change in the concentration of antibodies in the mouse serum is shown in FIG. 9D. The results showed that intramuscular immunization with the PP-3K-A5 complex vaccine in combination with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 17: Evaluation of the Immunogenicity of Phosphorylated Polypeptide Antigen A12 Coupled with a Norovirus P Protein in Wild Mice

Wild mice were intramuscularly immunized with the vaccine prepared by combining phosphorylated polypeptide antigen A12 coupled with PP-3K with AS02+CpG adjuvant. Each group contained 6 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1356) coupled with BSA was used as the coating antigen, and was phosphorylated at sites corresponding to amino acids 212, 214, 231 and 235 of full-length Tau protein. The change in the concentration of antibodies in the mouse serum is shown in FIG. 9E. The results showed that intramuscular immunization with the PP-3K-A12 complex vaccine in combination with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 18: Evaluation of the Immunogenicity of Phosphorylated Polypeptide Antigen A12 Coupled with a Norovirus P Protein in P301S Transgenic Mice

P301S transgenic mice were intramuscularly immunized with the vaccine prepared by combining phosphorylated polypeptide antigen A12 coupled with PP-3K with AS02+CpG adjuvant. Each group contained 12 mice. The immune dosage of the protein was 25 μg/animal. The immunization was carried out at week 0, week 2, week 4, week 6 and week 12. Blood was taken two weeks after every immunization to obtain serum for ELISA assessment. In the ELISA experiments, the phosphorylated polypeptide (whose sequence is SEQ ID NO: 1356) coupled with BSA was used as the coating antigen, and was phosphorylated at sites corresponding to amino acids 212, 214, 231 and 235 of full-length Tau protein. The change in the concentration of antibodies in the mouse serum is shown in FIG. 9F. The results showed that intramuscular immunization with the PP-3K-A12 complex vaccine combined with AS02+CpG adjuvant led to an increase in antibodies with each immunization, the vaccine had good immunogenicity, and high antibody concentrations could be maintained for at least 6 weeks after the last immunization, with antibody titer ≥12,800.

### Example 19: Evaluation of the Cellular Immunity of Phosphorylated Polypeptide Antigen A5 Coupled with a Norovirus P Protein in P301S Transgenic Mice—ELISPOT Detection

A96-well plate was coated with the monoclonal antibody against cytokine interferon γ (from elispot kit purchased from BD Corporation) in a concentration of 5 μg/mL (50 μL/well), and covered at 4° C. overnight. After discarding the coating antibody and washing once with a complete medium containing 10% fetal bovine serum, 200 μL of this complete medium was added to each well. Blocking was carried out at 37° C. for 1 hour, and then the medium was discarded. Mice used in the experiment were sacrificed by neck-pulling. Spleen cells of the mice were taken out and formulated into a cell suspension in a cell concentration of 107/mL. The cell suspension was added to the coated 96-well plate (100 μL/well). 100 μL of 1 μg/mL prokaryotically expressed full-length Tau protein and nonphosphorylated A5 polypeptide antigen were added to each well. The plate was then cultured at 37° C. in an incubator containing 5% CO₂ for 24 h to stimulate and activate the cells. 24 h later, the plate was washed two times with sterile water, and six times with sterile PBST (pH7.4, 0.01 mol/L PBS, containing 0.05% Tween-20) buffer to wash the cells away. 50 μL of 2 μg/mL antibody against interferon γ (from elispot kit purchased from BD Corporation) was added to each well and incubated at room temperature for 2 hours. The 96-well plate was washed, and horse radish peroxidase labeled biotin secondary antibody (from elispot kit purchased from BD Corporation) was added (50 μL/well). The plate was cultured at room temperature for 2 h, and washed four times with PBST, and two times with PBS. 50 μL of Elispot color developing solution (AEC substrate) was added to each well and reacted in the dark at room temperature for 5-60 min. The staining solution was discarded, and the plate was washed with distilled water. After being dried overnight, the sample was calculated for the number of activated cells using a microscope.

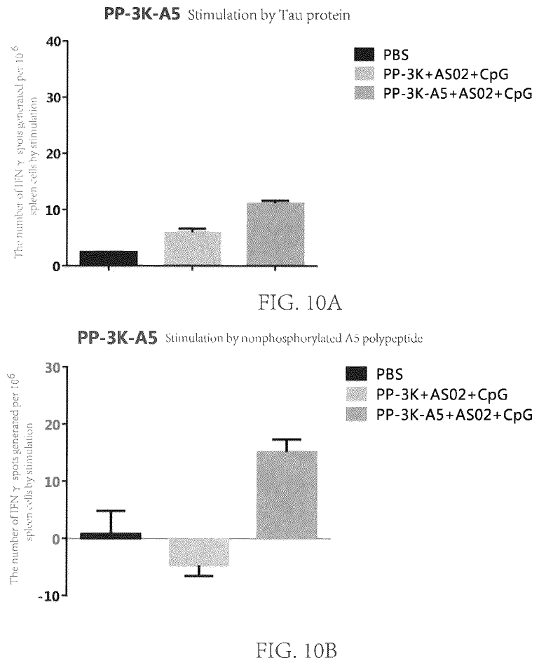

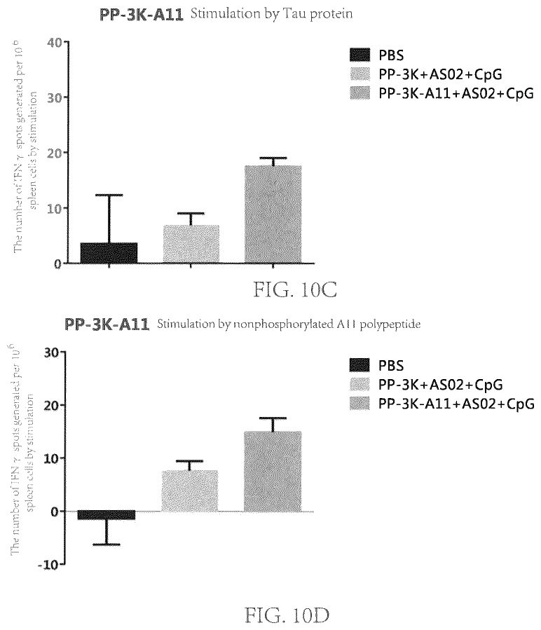

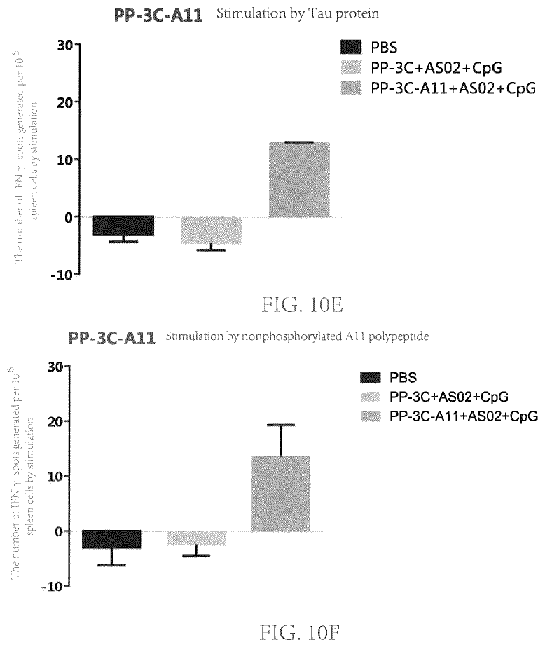

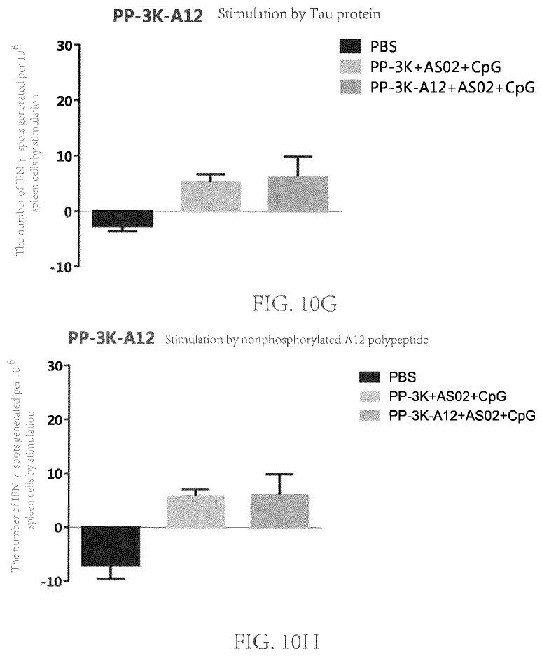

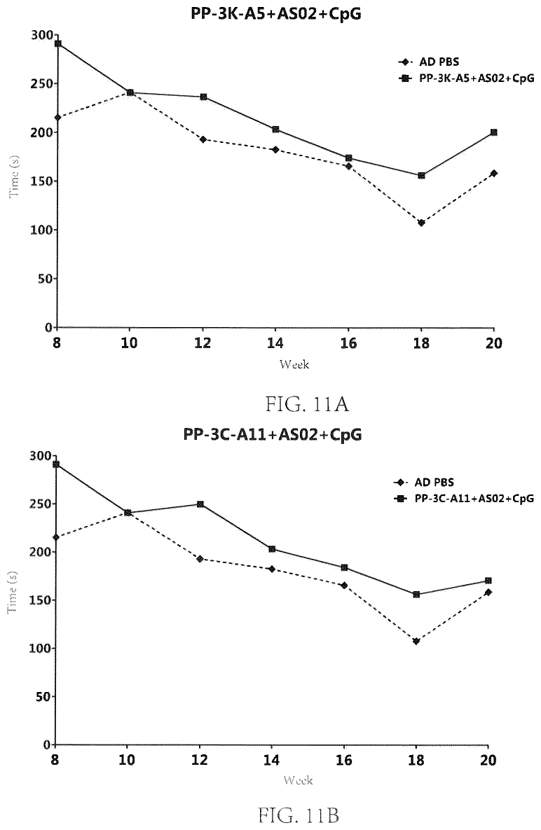

The experimental results are shown in FIGS. 10A and 10B. The PP-3K-A5 complex vaccine combined with AS02+CpG adjuvant could stimulate spleen cells to produce fewer spots after five immunizations, which demonstrates no evident T cell response occurring in the body, and as described in Example 14, this immunization strategy can stimulate the mice to produce the highest titer of specific antibodies against phosphorylated A5 polypeptide. Considering the safety of vaccines, this immunization strategy can be selected as a preferred immunization strategy.

### Example 20: Evaluation of the Cellular Immunity of Phosphorylated Polypeptide Antigen A11 Coupled with a Norovirus P Protein in P301S Transgenic Mice—ELISPOT Detection

The experiment was performed by the same method as in Example 19. The stimulus was 100 μL of 1 μg/mL prokaryotically expressed full-length Tau protein and nonphosphorylated A11 polypeptide antigen.

The experimental results are shown in FIGS. 10C and 10D. The PP-3K-A11 complex vaccine combined with AS02+CpG adjuvant could stimulate spleen cells to produce fewer spots after five immunizations, which demonstrates no evident T cell response occurring in the body, and as described in Example 15, this immunization strategy can stimulate the mice to produce the highest titer of specific antibodies against phosphorylated A11 polypeptide. Considering the safety of vaccines, this immunization strategy can be selected as a preferred immunization strategy.

### Example 21: Evaluation of the Cellular Immunity of Phosphorylated Polypeptide Antigen A11 Coupled with a Norovirus P Protein in P301S Transgenic Mice—ELISPOT Detection

The experiment was performed by the same method as in Example 19. The stimulus was 100 μL of 1 μg/mL prokaryotically expressed full-length Tau protein and nonphosphorylated A11 polypeptide antigen.

The experimental results are shown in FIGS. 10E and 10F. The PP-3K-A11 complex vaccine combined with AS02+CpG adjuvant could stimulate spleen cells to produce fewer spots after five immunizations, which demonstrates no evident T cell response occurring in the body, and as described in Example 15, this immunization strategy can stimulate the mice to produce the highest titer of specific antibodies against phosphorylated A11 polypeptide. Considering the safety of vaccines, this immunization strategy can be selected as a preferred immunization strategy.

### Example 22: Evaluation of the Cellular Immunity of Phosphorylated Polypeptide Antigen A12 Coupled with a Norovirus P Protein in P301S Transgenic Mice—ELISPOT Detection

The experiment was performed by the same method as in Example 19. The stimulus was 100 μL of 1 μg/mL prokaryotically expressed full-length Tau protein and nonphosphorylated A12 polypeptide antigen.

The experimental results are shown in FIGS. 10G and 10H. The PP-3K-A12 complex vaccine combined with AS02+CpG adjuvant could stimulate spleen cells to produce fewer spots after five immunizations, which demonstrates no evident T cell response occurring in the body, and as described in Example 15, this immunization strategy can stimulate the mice to produce the highest titer of specific antibodies against phosphorylated A12 polypeptide. Considering the safety of vaccines, this immunization strategy can be selected as a preferred immunization strategy.

### Example 23: Evaluation of the Behavior of P301S Transgenic Mice Immunized with Phosphorylated Polypeptide Antigen A5 Coupled with a Norovirus P Protein

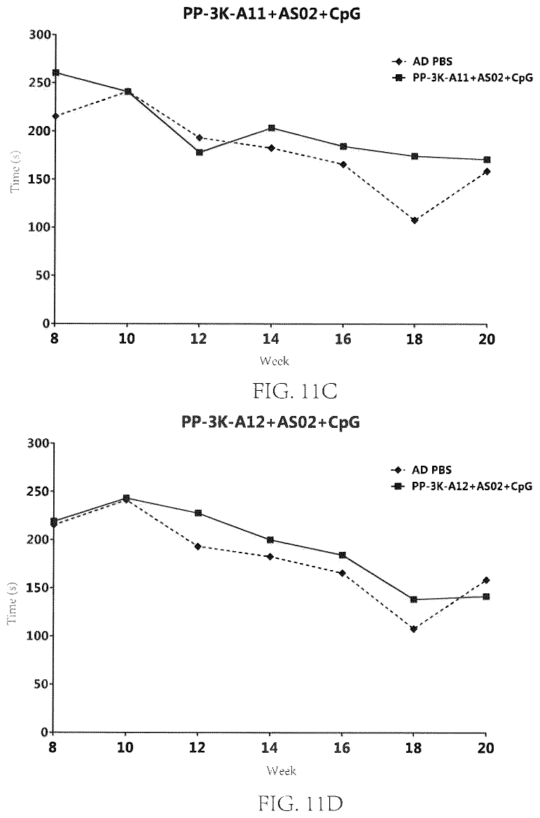

P301S transgenic mice were immunized with phosphorylated polypeptide antigen A5 which was coupled with PP-3K and combined with AS02+CpG adjuvant. Eight weeks after the start of the experiment, mice in the immune group were subjected to a rota-rod endurance test every two weeks. The mice were placed on the mouse rota-rod apparatus, and after the rota-rod apparatus was turned on, its rotation speed rose from 5 rpm to 40 rpm within 1.5 minutes and remained constant. The time until the mice fell from the rota-rod within 300 s was recorded. The experiment showed that the immunized mice had stronger endurance in the rota-rod experiment than the PBS group (the results are shown in FIG. 11A).

### Example 24: Evaluation of the Behavior of P301S Transgenic Mice Immunized with Phosphorylated Polypeptide Antigen A11 Coupled with a Norovirus P Protein

P301S transgenic mice were immunized with phosphorylated polypeptide antigen A11 which was coupled with PP-3C and combined with AS02+CpG adjuvant. Eight weeks after the start of the experiment, mice in the immune group were subjected to a rota-rod endurance test every two weeks. The mice were placed on the mouse rota-rod apparatus, and after the rota-rod apparatus was turned on, its rotation speed rose from 5 rpm to 40 rpm within 1.5 minutes and remained constant. The time until the mice fell from the rota-rod within 300 s was recorded. The experiment showed that the immunized mice had stronger endurance in the rota-rod experiment than the PBS group (the results are shown in FIG. 11B).

### Example 25: Evaluation of the Behavior of P301S Transgenic Mice Immunized with Phosphorylated Polypeptide Antigen A11 Coupled with a Norovirus P Protein

P301S transgenic mice were immunized with phosphorylated polypeptide antigen A11 which was coupled with PP-3K and combined with AS02+CpG adjuvant. Eight weeks after the start of the experiment, mice in the immune group were subjected to a rota-rod endurance test every two weeks. The mice were placed on the mouse rota-rod apparatus, and after the rota-rod apparatus was turned on, its rotation speed rose from 5 rpm to 40 rpm within 1.5 minutes and remained constant. The time until the mice fell from the rota-rod within 300 s was recorded. The experiment showed that the immunized mice had stronger endurance in the rota-rod experiment than the PBS group (the results are shown in FIG. 11C).

### Example 26: Evaluation of the Behavior of P301S Transgenic Mice Immunized with Phosphorylated Polypeptide Antigen A12 Coupled with a Norovirus P Protein

P301S transgenic mice were immunized with phosphorylated polypeptide antigen A12 which was coupled with PP-3K and combined with AS02+CpG adjuvant. Eight weeks after the start of the experiment, mice in the immune group were subjected to a rota-rod endurance test every two weeks. The mice were placed on the mouse rota-rod apparatus, and after the rota-rod apparatus was turned on, its rotation speed rose from 5 rpm to 40 rpm within 1.5 minutes and remained constant. The time until the mice fell from the rota-rod within 300 s was recorded. The experiment showed that the immunized mice had stronger endurance in the rota-rod experiment than the PBS group (the results are shown in FIG. 11D).

### Example 27: Evaluation of the Behavior of P301S Transgenic Mice Immunized with Phosphorylated Polypeptide Antigen A5 Coupled with a Norovirus P Protein

P301S transgenic mice were immunized with phosphorylated polypeptide antigen A5 couplded to PP-3K and combined with AS02+CpG adjuvant. Ten weeks after the start of the experiment, a nesting test was performed. All mice were transferred to new sterile mouse boxes. New sterile bedding was spread evenly to a thickness of 0.8 cm/box. Two 5 cm×5 cm cotton pieces were placed in the center of the right side of each mouse box, the total weight of the cotton being 1.2 g to 1.3 g. The mice were transferred to the mouse boxes at 16:00 and the indoor lighting was turned off. The lighting was turned on at 7:00 the next day and the nesting effect of mice was scored at 8:00. The labels with mouse numbers were hidden and three trained operators performed the scoring independently of each other. The average of the three scores represents the score of one mouse in the nesting test.

The scoring criteria were as follows: 1: the cotton was not completely torn; 2: the cotton was completely torn for nest building, the nest being shallow and flat; 3: the cotton was completely torn for nest building, but the nest had other notches besides those for exposing drinking water and the like, or the upper edge of the nest entrance was lower than the mouse's head; 4: the cotton was completely torn for nest building, and the nest entrance was higher than the mouse's head, or the nest was three-dimensional and had no notch.

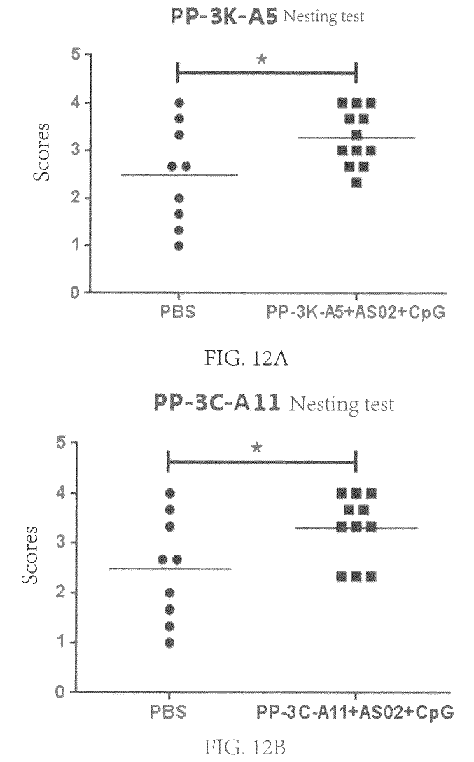

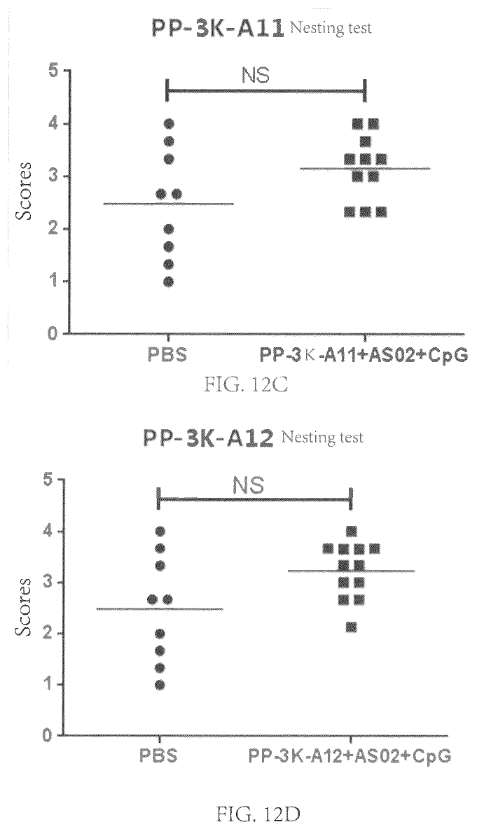

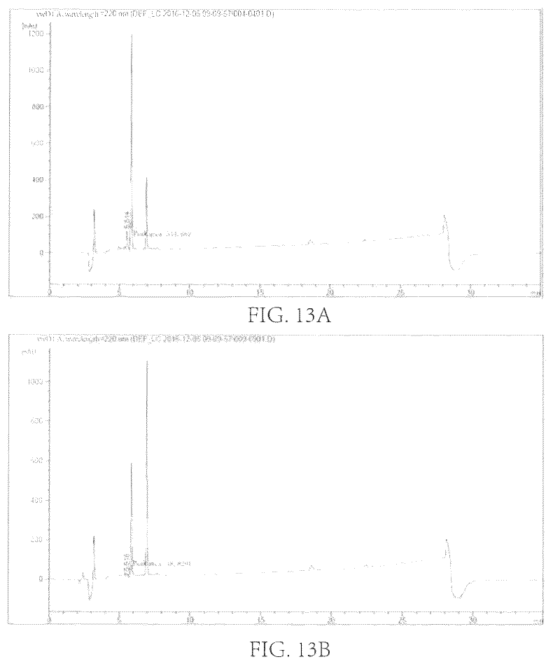

The nesting test can reflect the integrity of the consciousness of mice. The experiment showed that the immunized mice performed significantly better than the PBS group in the nesting test (the results are shown in FIG. 12A).

### Example 28: Evaluation of the Behavior of P301S Transgenic Mice Immunized with Phosphorylated Polypeptide Antigen A11 Coupled with a Norovirus P Protein

P301S transgenic mice were immunized with phosphorylated polypeptide antigen A11 coupled with PP-3C and combined with AS02+CpG adjuvant. Ten weeks after the start of the experiment, a nesting test was performed by the same method as in Example 27. The experiment showed that the immunized mice performed significantly better than the PBS group in the nesting test (the results are shown in FIG. 12B).

### Example 29: Evaluation of the Behavior of P301S Transgenic Mice Immunized with Phosphorylated Polypeptide Antigen A11 Coupled with a Norovirus P Protein

P301S transgenic mice were immunized with phosphorylated polypeptide antigen A11 coupled with PP-3K and combined with AS02+CpG adjuvant. Ten weeks after the start of the experiment, a nesting test was performed by the same method as in Example 27. The experiment showed that the immunized mice performed significantly better than the PBS group in the nesting test (the results are shown in FIG. 12C).

### Example 30: Evaluation of the Behavior of P301S Transgenic Mice Immunized with Phosphorylated Polypeptide Antigen A12 Coupled with a Norovirus P Protein

P301S transgenic mice were immunized with phosphorylated polypeptide antigen A12 coupled with PP-3K and combined with AS02+CpG adjuvant. Ten weeks after the start of the experiment, a nesting test was performed by the same method as in Example 27. The experiment showed that the immunized mice performed significantly better than the PBS group in the nesting test (the results are shown in FIG. 12D).

### Example 31: Construction of pET28a-C-PA Plasmid

The plasmid containing C-PA was obtained through sequence optimization and gene synthesis (GL Bio).

The C-PA genes were enriched by PCR method and the sequence of the genes was represented by SEQ ID NO: 1363. The specific method was as follows:

SEQ ID NO: 1365 (forward):
TTTAACTTTAAGAAGGAGATATACATATGGGTGGTGGTGGTTCTTGCGG
CGGCG,
and
SEQ ID NO: 1366 (reverse):
GTGGTGGTGGTGGTGGTGCTCGAGTTATTTAATACGCAGATACTGGCCA
ATCA;

The plasmid containing C-PA was subjected to PCR reaction, wherein the PCR reaction system was a KOD-Plus DNA polymerase system (purchased from TOYOBO Corporation), and the total volume of the reaction system was 50 μL (5 L buffer, 0.2 mM dNTP, 1 mM magnesium sulfate, 0.3 μM upstream primer and 0.3 μM downstream primer, 50 ng template DNA, 1 μL KOD enzyme, and water which was added to a final volume of 50 μL). PCR was carried out in accordance with the reaction system instructions to obtain 50 μL of PCR products. The products were subjected to agarose gel electrophoresis, and the fragments of interest were recovered using gel recovery kit (purchased from Tiangen Biotech Co., Ltd.)

1 μL of BamHI enzyme and 1 μL of XhoI enzyme (purchased from Takara Corporation) and 5 μL of enzyme digestion buffer (purchased from Takara Corporation) were added respectively to 2 μg of pET28a vector plasmid (purchased from Novagen Corporation), and finally sterile water was added to the system to reach a final volume of 50 μL. Digestion was carried out at 37° C. for 2 hours. The products were subjected to agarose gel electrophoresis, and the vector fragments were recovered using gel recovery kit (purchased from Tiangen Biotech Co., Ltd.) to obtain a linear plasmid vector.

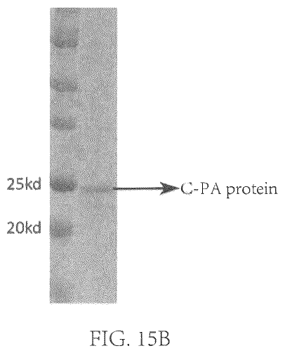

The above double enzyme-digested vector fragment and fragment of interest (with a molar ratio of 1:3, and a total volume of 5 μL) were mixed, and 5 μL of homologous recombination mix (purchased from Taihe Corporation) was added. The ligation was carried out at 25° C. for 15 min. 10 μL of the ligated products were added to DH10B competent cells (purchased from Taihe Corporation), and placed on ice for 30 min. After that, the cells were subjected to heat shock at 42° C. for 30 s, placed on ice for 2 min, and then were added to 600 L of fluid LB medium without resistance, and recovered at 200 rpm at 37° C. for 1 h. The culture broth was plated on a LB solid culture plate containing kanamycin (15 μg/mL), and were placed upside down at 37° C. overnight. The recombinant clones were obtained, and the sequence was verified by sequencing, which was represented by SEQ ID NO: 1363. A pET28a-C-PA plasmid that can express C-PA protein was obtained, as shown in FIG. 15A.

### Example 32: Expression and Purification of a C-PA Protein

### 32.1 Expression of a C-PA Protein

1 μL of the recombinant plasmids prepared in the above examples were respectively added to 100 μL of E. coli BL21 competent cells (purchased from TransGen Corporation), ice-bathed for 30 min, heat shocked for 90 s in a water-bath at 42° C., and then ice-bathed for 2 min. 600 μL of LB medium was added to the mixture, and cultured at 180 rpm/min at 37° C. for 1 h. The mixture was coated evenly on a LB solid medium containing kanamycin (15 μg/mL) resistance and cultured at 37° C. for 24 h to obtain strains that can stably express recombinant proteins. A growing colony was picked and inoculated into 20 mL of LB medium. The mixture was cultured at 220 rpm at 37° C. When the OD value of the culture mixture reached 2.0, induction by Isopropyl β-D-Thiogalactoside (IPTG at a final concentration of 1.0 mmol/L) was carried out at 220 rpm at 37° C. for six hours. After the induction, the culture broth was centrifuged at 4,000 rpm for 20 min. The supernatant was discarded, and the bacterial precipitates were resuspended with PBS. Centrifugation was conducted again at 4,000 rpm for 20 min and the supernatant was discarded to obtain the bacterial precipitates containing proteins of interest.

### 32.2 Purification of a C-PA Protein

The bacterial precipitates obtained in 32.1 were resuspended by adding 20 mL of protein buffer (pH=7.0, containing 50 mM Tris and 300 mM KCl). The bacteria were lysed by sonication on ice for 30 min. The mixture was centrifuged at 12,000 rpm at 4° C. for 30 min and the supernatant was discarded. The collected precipitates were resuspended in 8M urea buffer (pH=7.0, containing 50 mM Tris, 300 mM KCl and 8M urea) and stirred overnight at 4° C. The collected solution was centrifuged at 16,000 rpm at 4° C. for 30 min. The supernatant was collected and transferred to a dialysis bag with a cut-off size of 10 kd. Dialysis was performed in the ratio of the volume of glycine dialysate to the volume of protein solution of 10:1, and the fluid was changed every 4 hours. The dialysis was performed 6 times to slowly renature the protein. The collected protein solution was concentrated by ultrafiltration to a protein concentration of 1.0 mg/mL to 2.0 mg/mL. The results of SDS-PAGE of the protein are shown in FIG. 15B, and the molecular weight of the protein is about 24 kd.

### Example 33: Coupling Reaction of Phosphorylated Polypeptide Antigen Vaccines with BLP

### Example 33.1: Ligation Reaction of C-PA with BLP

The method for preparing BLP was described in the Chinese Patent Application CN 105968213. 1 mL of 15 mg/mL BLP solution was centrifuged at 5,000 g at 4° C. for 30 min, and the supernatant was discarded. BLP was resuspended with 5 mL of C-PA solution and slowly shaked at 25° C. for 30 min to prepare C-PA-BLP. Centrifuge was performed at 5,000 g for 30 min at 4° C., the supernatant was discarded, and resuspension was performed with 10 mL sterile PBS (pH=7.2). This operation was repeated 4 times. The washed precipitates were resuspended with 1 mL PBS, OD600 was detected, and the concentration of C-PA-BLP was calibrated with a BLP standard.

### Example 33.2: Ligation Reaction of C-PA with BLP

The method for preparing BLP was described in the Chinese Patent Application CN 105968213. 1 mL of 15 mg/mL BLP solution was centrifuged at 5,000 g at 4° C. for 30 min, and the supernatant was discarded. BLP was resuspended with 3 mL of C-PA solution and slowly shaked at 4° C. for 6 h to prepare C-PA-BLP. Centrifuge was performed at 5,000 g for 30 min at 4° C., the supernatant was discarded, and resuspension was performed with 10 mL sterile PBS (pH=7.2). This operation was repeated 4 times. The washed precipitates were resuspended with 1 mL PBS, OD600 was detected, and the concentration of C-PA-BLP was calibrated with a BLP standard.

### Example 33.3: Coupling Reaction of the Phosphorylated Polypeptide Antigen Vaccines with C-PA-BLP

The phosphorylated polypeptide in each of the phosphorylated polypeptide antigen vaccines in lyophilized form prepared in Example 1 was diluted with a 0.1 M ammonium bicarbonate solution (pH=7.5) to a concentration of 1 mg/mL and was quantified. In the ratio of the molar amount of the C-PA protein to the molar amount of the polypeptide of 1:30, the polypeptide solution was slowly added to the precipitates obtained after centrifugation of C-PA-BLP, and mixed slowly. The mixed system was incubated at 2° C. to 8° C. with slow shaking for 48 hours. The obtained product was centrifuged and resuspended with PBS to remove unbound polypeptide.

### Example 33.4: Coupling Reaction of the Phosphorylated Polypeptide Antigen Vaccines with C-PA-BLP

The phosphorylated polypeptide in each of the phosphorylated polypeptide antigen vaccines in lyophilized form prepared in Example 1 was diluted with a 0.1 M ammonium bicarbonate solution (pH=8.0) to a concentration of 2 mg/mL and was quantified. In the ratio of the molar amount of the C-PA protein to the molar amount of the polypeptide of 1:100, the polypeptide solution was slowly added to the precipitates obtained after centrifugation of C-PA-BLP, and mixed slowly. The mixed system was incubated at 2° C. to 8° C. with slow shaking for 48 hours. The obtained product was centrifuged and resuspended with PBS to remove unbound polypeptide.

### Example 33.5: Coupling Reaction of the Phosphorylated Polypeptide Antigen Vaccines with C-PA-BLP

The phosphorylated polypeptide in each of the phosphorylated polypeptide antigen vaccines in lyophilized form prepared in Example 1 was diluted with a 0.1 M ammonium bicarbonate solution (pH=8.0) to a concentration of 2 mg/mL and was quantified. In the ratio of the molar amount of the C-PA protein to the molar amount of the polypeptide of 1:100, the polypeptide solution was slowly added to the precipitates obtained after centrifugation of C-PA-BLP, and mixed slowly. The mixed system was incubated at 2° C. to 8° C. with slow shaking for 48 hours. The obtained product was centrifuged and resuspended with PBS to remove unbound polypeptide.

### Example 34: Determination of the Ligation Efficiency of the Phosphorylated Polypeptide Antigen Product Coupled with BLP

A10 mM dithiothreitol solution was added to the phosphorylated polypeptide antigen product coupled with C-PA-BLP at a volume ratio of 1:1, and mixed homogeneously, and the mixture was left to stand at room temperature to react for 16 hours. The reaction product was centrifuged at 16,000 g at 4° C. for 15 min. The supernatants were taken for HPLC determination. Using 0.5 mg/mL, 0.25 mg/ml, 0.125 mg/ml, 0.0625 mg/ml, 0.03125 mg/mL, 0.03125 mg/mL, and 0.015625 mg/mL phosphorylated polypeptide dissolved in 10 mM of a dithiothreitol solution as standard, a standard curve was plotted with the concentration of the standard versus the peak area of the phosphorylated peptide to quantitatively analyze the concentration of the test sample (the results are shown in FIGS. 13A, 13C and 13D).

It should be understood that specific embodiments described herein are only used for explaining the present invention, instead of limiting the present invention. The protection scope of the present invention is subject to the protection scope defined in claims. For those of ordinary skill in the art, a variety of changes and modifications can be made without departing from the spirit and scope of the present invention, and these changes and modifications should be considered to belong to the protection scope of the present invention.

1-21. (canceled)

22. A phosphorylated polypeptide antigen vaccine, which comprises at least two polypeptide fragments or conservatively modified variants thereof from human full-length Tau protein, wherein the polypeptide fragments or conservatively modified variants thereof contain phosphorylation sites; preferably, the phosphorylated polypeptide antigen vaccine contains an additional cysteine residue at its C′-terminal.

23. The phosphorylated polypeptide antigen vaccine of claim 22, wherein the polypeptide fragments are connected directly by peptide bonds or connected by amino acid linkers; preferably, the polypeptide fragments are connected directly by peptide bonds.

24. The phosphorylated polypeptide antigen vaccine of claim 22, wherein the polypeptide fragments are derived from phosphorylation modification site-rich regions of human full-length Tau protein; preferably, the polypeptide fragments are derived from regions of human full-length Tau protein as follows: amino acids at positions 14 to 22 of human full-length Tau protein, amino acids at positions 194 to 266 of human full-length Tau protein, and/or amino acids at positions 392 to 408 of human full-length Tau protein.

25. The phosphorylated polypeptide antigen vaccine of claim 24, wherein the phosphorylation sites include two or more, preferably all phosphorylated amino acid sites corresponding to positions 18, 202, 205, 212, 214, 231, 235, 238, 262, 396 and 404 of the amino acid sequence of human full-length Tau protein, namely, 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄); more preferably, 1 to 4, preferably 1 to 3, more preferably 1 to 2, and most preferably 1 Ser site of the phosphorylation sites are substituted by an aspartic acid and/or 1 to 4, preferably 1 to 3, more preferably 1 to 2, and most preferably 1 Thr site of the phosphorylation sites are substituted by a glutamic acid.

26. The phosphorylated polypeptide antigen vaccine of claim 22, wherein the conservatively modified variant of the polypeptide fragment is a variant obtained by conservatively substitution of one or more amino acids, preferably 1 to 10 amino acids, more preferably 1 to 6 amino acids, more preferably 1 to 4 amino acids, more preferably 1 to 3 amino acids and most preferably 1 amino acid of the polypeptide fragment with functionally similar amino acids.

27. The phosphorylated polypeptide antigen vaccine of claim 22, which has an amino acid sequence as represented by any one of SEQ ID NOs: 1-1331 and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄);
preferably, the phosphorylated polypeptide antigen vaccine has an amino acid sequence which has at least 80%, at least 85%, at least 90%, at least 95%, preferably at least 98%, and more preferably at least 99% sequence identity to any one of SEQ ID NOs: 1-1331; more preferably, the phosphorylated polypeptide antigen vaccine has an amino acid sequence as represented by any one of SEQ ID NO: 201, SEQ ID NO: 225, SEQ ID NO: 306, SEQ ID NO: 387, SEQ ID NO: 468, SEQ ID NO: 558, SEQ ID NO: 567, SEQ ID NO: 769, SEQ ID NO: 784, SEQ ID NO: 875, SEQ ID NO: 1020, SEQ ID NO: 1101, SEQ ID NO: 1182, SEQ ID NO: 1272, SEQ ID NO: 1313 and SEQ ID NO: 1330, and the amino acid sequence contains two or more phosphorylation sites selected from 18(P-Tyr₁₈), 202(P-Ser₂₀₂), 205(P-Thr₂₀₅), 212(P-Thr₂₁₂), 214(P-Ser₂₁₄), 231(P-Thr₂₃₁), 235(P-Ser₂₃₅), 238(P-Ser₂₃₈), 262(P-Ser₂₆₂), 396(P-Ser₃₉₆) and 404(P-Ser₄₀₄); and most preferably, the phosphorylated polypeptide antigen vaccine has an amino acid sequence as represented by any one of SEQ ID NO: 201, SEQ ID NO: 225, SEQ ID NO: 306, SEQ ID NO: 387, SEQ ID NO: 468, SEQ ID NO: 558, SEQ ID NO: 567, SEQ ID NO: 769, SEQ ID NO: 784, SEQ ID NO: 875, SEQ ID NO: 1020, SEQ ID NO: 1101, SEQ ID NO: 1182, SEQ ID NO: 1272, SEQ ID NO: 1313 and SEQ ID NO: 1330, and the amino acid sequence respectively contain phosphorylation sites as follows: 18(P-Tyr₁₈), 202(P-Ser₂₀₂), 205(P-Thr₂₀₅); 18(P-Tyr₁₈), 202(P-Ser₂₀₂), 205(P-Thr₂₀₅); 18(P-Tyr₁₈), 212(P-Thr₂₁₂), 214(P-Ser₂₁₄); 18(P-Tyr₁₈), 231(P-Thr₂₃₁), 235(P-Ser₂₃₅); 18(P-Tyr₁₈), 238(P-Ser₂₃₈), 262(P-Ser₂₆₂); 18(P-Tyr₁₈), 396(P-Ser₃₉₆), 404(P-Ser₄₀₄); 202(P-Ser₂₀₂), 205(P-Thr₂₀₅), 231(P-Thr₂₃₁), 235(P-Ser₂₃₅); 202(P-Ser₂₀₂), 205(P-Thr₂₀₅), 231(P-Thr₂₃₁), 235(P-Ser₂₃₅); 202(P-Ser₂₀₂), 205(P-Thr₂₀₅), 238(P-Ser₂₃₈), 262(P-Ser₂₆₂); 202(P-Ser₂₀₂), 205(P-Thr₂₀₅), 396(P-Ser₃₉₆), 404(P-Ser₄₀₄); 202(P-Ser₂₀₂), 205(P-Thr₂₀₅), 396(P-Ser₃₉₆), 404(P-Ser₄₀₄); 212(P-Thr₂₁₂), 214(P-Ser₂₁₄), 231(P-Thr₂₃₁), 235(P-Ser₂₃₅); 212(P-Thr₂₁₂), 214(P-Ser₂₁₄), 238(P-Ser₂₃₈), 262(P-Ser₂₆₂); 212(P-Thr₂₁₂), 214(P-Ser₂₁₄), 396(P-Ser₃₉₆), 404(P-Ser₄₀₄); 238(P-Ser₂₃₈), 262(P-Ser₂₆₂), 396(P-Ser₃₉₆), 404(P-Ser₄₀₄); 238(P-Ser₂₃₈), 262(P-Ser₂₆₂), 396(P-Ser₃₉₆) and 404(P-Ser₄₀₄).

28. A complex vaccine formed by coupling the phosphorylated polypeptide antigen vaccine of claim 22 with a carrier.

29. The complex vaccine of claim 28, wherein the carrier is selected from the group consisting of human serum albumin, keyhole limpet hemocyanin, bacterium-like particles (BLP), immunoglobulin molecules, thyroglobulin, ovalbumin, bovine serum albumin component V, influenza hemagglutinin, PAN-DR binding peptide (PADRE polypeptide), malaria circumsporozoite (CS) protein, hepatitis B surface antigen (HBₛAg₁₉₋₂₈), Heat Shock Protein (HSP) 65, Bacille Calmette-Guérin (BCG), cholera toxin, attenuated cholera toxin variants, diphtheria toxin, norovirus capsid P protein, recombinant Streptococcus C5a peptidase, Streptococcus pyogenes ORF1224, Streptococcus pyogenes ORF1664, Streptococcus pyogenes ORF2452, pneumolysin, attenuated pneumolysin toxicity variants, Chlamydia pneumoniae ORFT367, Chlamydia pneumoniae ORFT858, Tetanus toxoid, HIV gp120T1, microbial surface components recognizing adhesive matrix molecules (MSCRAMMS), growth factor/hormone and/or chemokines;
preferably, the carrier is bacterium-like particles (BLP); and more preferably, the BLP is coupled with a phosphorylated polypeptide antigen vaccine by means of a protein adaptor; and most preferably, the protein adaptor has a sequence as represented by SEQ ID NO: 1364; preferably, the carrier is norovirus capsid P protein; and more preferably, the norovirus capsid P protein is PP-3C with a sequence as represented by SEQ ID NO: 1357 or PP-3K with a sequence as represented by SEQ ID NO: 1359.

30. A method for preparing the complex vaccine of claim 28, comprising the following steps:
1) artificially synthesizing the phosphorylated polypeptide antigen vaccine of claim 22;
2) preparing a carrier to be coupled to the phosphorylated polypeptide antigen vaccine;
3) mixing the phosphorylated polypeptide antigen vaccine with the carrier to perform coupling reaction; and
4) separating and purifying the conjugate obtained in 3), thereby obtaining a complex vaccine.

31. The method of claim 30, wherein the carrier in step 2) is a norovirus capsid P protein, preferably PP-3C or PP-3K, and step 2) specifically comprises:
i) obtaining an expression vector comprising a nucleic acid encoding a PP-3C or PP-3K protein;
ii) transferring the expression vector into a receptor cell;
iii) expressing the PP-3C or PP-3K protein, and allowing it to self-assemble into a recombinant P particle in the receptor cell;
preferably, step 2) also comprises isolation and purification steps; and more preferably, ion exchange chromatography and/or hydrophobic chromatography are used for purification.

32. The method of claim 30, wherein in step 3), PP-3C is used as a vaccine carrier for coupling, a preferred buffer system is an ammonium bicarbonate buffer system, and a preferred pH ranges from 7.5 to 8.8; preferably, the phosphorylated polypeptide antigen vaccine and the carrier are mixed in a ratio of 10:1 to 100:1, and a preferred reaction temperature ranges from 2° C. to 10° C.; or in step 3), PP-3K is used as a vaccine carrier for coupling, and a preferred buffer system is a phosphate buffer system, and a preferred pH ranges from 7.0 to 8.5; preferably, the phosphorylated polypeptide antigen vaccine and the carrier are mixed in a ratio of 10:1 to 100:1, and a preferred reaction temperature ranges from 2° C. to 25° C.

33. The method of claim 30, wherein the carrier in step 2) is bacterium-like particles (BLP), and step 3) specifically comprises:
i) obtaining a purified protein adaptor-C-PA protein with a sequence as represented by SEQ ID NO: 1364; and
ii) connecting the carrier—bacterium-like particles (BLP)—obtained in step 2) with the C-PA protein to obtain C-PA-BLP; and iii) preforming coupling reaction of C-PA-BLP with the phosphorylated polypeptide antigen vaccine;
wherein a Tris buffer system is used as a buffer system and a preferred pH ranges from 7.2 to 8.8; a preferred C-PA protein concentration is 0.1 mg/ml to 1.5 mg/mL, and a preferred reaction temperature ranges from 2° C. to 30° C.; or an ammonium bicarbonate buffer system is used as a buffer system, and a preferred pH ranges from 7.5 to 8.8; preferably, the phosphorylated polypeptide antigen vaccine and the C-PA-BLP are mixed in a ratio of 10:1 to 100:1, and a preferred reaction temperature ranges from 2° C. to 10° C.

34. The method of claim 30, wherein step 4) comprises removing coupling agent and polypeptide antigens which are not successfully connected by methods including desalting chromatography, dialysis and ultrafiltration.

35. A vaccine composition, wherein the vaccine composition comprises the phosphorylated polypeptide antigen vaccine of claim 22 or a complex vaccine; preferably, the vaccine composition further comprises a pharmaceutically acceptable adjuvant; more preferably, the pharmaceutically acceptable adjuvant is selected from one or more of CpG, MF59, AS02, AS03, Freund's complete adjuvant and Freund's incomplete adjuvant;
wherein the complex vaccine is formed by coupling the phosphorylated polypeptide antigen vaccine of claim 22 with a carrier.

36. Use of the phosphorylated polypeptide antigen vaccine of claim 22 or a complex vaccine or a vaccine composition for preparing a medicament for prevention and/or treatment of neurodegenerative disorders; and/or for preparing a medicament for maintaining or improving, preferably recovering, and more preferably completely recovering the cognitive memory of mammals, especially human beings;
wherein the complex vaccine is formed by coupling the phosphorylated polypeptide antigen vaccine of claim 22 with a carrier;
wherein the vaccine composition comprises the phosphorylated polypeptide antigen vaccine of claim 22 or the complex vaccine; preferably, the vaccine composition further comprises a pharmaceutically acceptable adjuvant; more preferably, the pharmaceutically acceptable adjuvant is selected from one or more of CpG, MF59, AS02, AS03, Freund's complete adjuvant and Freund's incomplete adjuvant.

37. Use of claim 36, wherein the neurodegenerative disorders are selected from one or more of AD, Creutzfeldt-Jacob Syndrome, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism syndrome-dementia syndrome, argyrophilic grain dementia, corticobasal degeneration, diffuse neurofibrillary tangles with calcification, frontotemporal dementia with parkinsonism syndrome linked to chromosome 17, Hallevorden-Spatz disease, multiple system atrophy, Niemann-Pick disease type C, Pick's disease, progressive subcortical gliosis and progressive supranuclear panencephalitis; preferably the neurodegenerative disorder is AD; preferably, the vaccine or vaccine composition is preferably immunized subcutaneously, intraperitoneally or intramuscularly, and more preferably immunized intramuscularly.
